(19) United States
(12) Patent Application Publication
(10) Pub. No.: US 2015/0031892 A1
(43) Pub. Date: Jan. 29, 2015

(54) SPHINGOLIPID METABOLITE MIMETICS

(71) Applicants: Zhejiang University, Hangzhou (CN); Georgia Regents Research Institute, Inc., Augusta, GA (US)

(72) Inventors: Feiyan Liu, Hangzhou (CN); Kebin Liu, Augusta, GA (US); Zhizhen Huang, Hangzhou (CN); Ping Wu, Hangzhou (CN)

(21) Appl. No.: 14/511,296

(22) Filed: Oct. 10, 2014

Related U.S. Application Data
(63) Continuation of application No. PCT/US2013/066576, filed on Oct. 24, 2013.
(60) Provisional application No. 61/718,916, filed on Oct. 26, 2012.

(51) Int. Cl. C07C 237/22; C07D 209/42; C07D 333/38; C07D 307/85; C07D 307/68; C07D 217/26

(52) U.S. Cl. CPC C07C 237/22; C07D 307/68; C07D 217/26; C07D 333/38; C07D 307/85; C07D 209/42
USPC 546/146; 554/56; 564/158; 549/487; 564/159; 564/155; 549/72; 549/467; 548/492

(57) ABSTRACT

Sphingolipid metabolite mimetics and methods of synthesizing them are provided. The sphingolipid metabolite mimetics are shown to be effective at inducing apoptosis in various types of tumor cells. Further, the sphingolipid metabolite mimetics are shown to be effective at sensitizing multiple types of tumor cells to TRAIL-induced apoptosis. Formulations containing one or more sphingolipid metabolite mimetics and, optionally, one or more cell death receptor agonists are provided. Methods of treating cancer in a subject in need thereof are provided using one or more sphingolipid metabolite mimetics.

### CROSS-REFERENCE TO RELATED APPLICATION

This application is a continuation of International Application No. PCT/US2013/066576 filed under the Patent Cooperation Treaty on Oct. 14, 2013, which claims benefit of and priority to U.S. Provisional Patent Application No. 61/718,916 filed Oct. 26, 2012, all of which are incorporated herein by reference in their entirety.

### STATEMENT REGARDING FEDERALLY SPONSORED RESEARCH OR DEVELOPMENT

This invention was made with Government Support under Agreement R01 CA133085 awarded to Kebin Liu by the National Institutes of Health. The Government has certain rights in the invention.

### FIELD OF THE INVENTION

The invention is generally related to mimetics of sphingolipid metabolites and methods of their use.

### BACKGROUND OF THE INVENTION

Cancer remains the second most common cause of death in the US, accounting for approximately 1 in every 4 deaths. The American Cancer Society estimates there will be approximately 1.6 million new cancer cases in the United States in 2012, and while markedly improved from previous years the 5-year survival rate remains only about 67%. Ideally, cancer therapies would be both effective in killing cancer cells and minimally toxic to non-cancer cells (Hall M A and Cleveland J L. Cancer Cell 2007; 12(1):4-6)). In reality, cancer cell resistance to chemotherapeutic drugs is a major problem limiting the effectiveness of many therapies used to treat human cancer (Longley D B and Johnston P G. J Pathol 2005; 205(2):275-92).

Cancer cells may be intrinsically resistant to chemotherapeutic drugs or may acquire resistance during treatment as a result of drug selection pressure on cancer cells. Drug resistance, whether intrinsic or acquired, is believed to account for treatment failure in over 90% of patients with metastatic cancer (Longley D B and Johnston P G. Journal of Pathology 2005; 205(2):275-92)). Therefore, finding ways to overcome drug resistance, particularly in metastatic cancers, may greatly improve the currently disappointing survival rate of patients with cancer. Essentially all cytotoxic anticancer drugs currently in clinical use or in clinical trials kill cancer cells through inducing apoptosis (Reed J C. Cancer Cell 2003, 3(1):17-22). Thus, tumor cell resistance to apoptosis, whether intrinsic or acquired, represents a major challenge in chemotherapeutic intervention of cancer, especially metastatic cancer.

Sphingolipids are bioeffectors known to mediate various cellular processes, including proliferation and apoptosis of cancer cells (Aoyagi, T. et al. Lymphatic Research and Biology 2012, 10:97-106; Apraiz, A. et al. BMC Cancer 2011, 11:477; Barcelo-Coblijn et al. Proceedings of the National Academy of Science USA 2011, 108(49):19569-19574; Berra et al. European Journal of Cancer Prevention, 2002, 11(2):193-197; Bini et al. Neuropharmacology, 2012, 63(4) 524-537; Chatzakos et al. Biochemical Pharmacology, 2012, 84(5):712-721; Duan and Nilsson Progress in Lipid Researhc, 2009, 48(1):62-72; Elojeimy et al. Molecular Therapeutics, 2007; 15:1259-1263; Furuya et al. Cancer Metastasis Reviews, 2011, 30:567-576; Gault et al. Journal of Biological Chemistry, 2012, 287:31794-31803; Kamocki et al. American Journal of Respiratory Cell and Molecular Biology, 2012; Kent et al. Lipids, 2008, 43:143-149; Kolesnick and Fuks Oncogene, 2003, 22:5897-5906; Taniguchi M. Journal of Biological Chemistry, 2012; Korbelik et al. Photochemical and Photobiological Sciences, 2012; Kumar et al. Carcinogenesis, 2012, 33:291-295; Morad et al. Molecular Cancer Therapeutics, 2012; Morales and Fernandez-Checa Mini-Reviews in Medicinal Chemistry, 2007, 7:371-382; Ogretmen B. FEBS Letters, 2006, 580:5467-5476; Oskouian et al. Proceedings of the National Academy of Science USA, 2006; 103: 17384-17389; Patwardhan and Liu Progress in Lipid Research, 2011, 50:104-114; Perry and Kolesnick Cancer Treatment Research, 2003; 115:345-354; Ponnusamy et al. Future Oncology, 2010, 6:1603-1624; Radin N. S. Urology, 2002, 60:562-568; Radin N. S. Cancer Investigation, 2002, 20:779-786; Russo et al. Journal of Clinical Investigations, 2012; Saddoughi et al. Subcellular Biochemistry, 2008, 49:413-440; Santos and Schulze FEBS Journal, 2012, 279:2610-2623; Schmelz Frontiers in Bioscience, 2004, 9:2632-2639; Segui et al. Biochemica et Biophysica Acta, 2006, 1758:2104-2120; Senkal et al. Faseb Journal, 2010, 24:296-308; Simon et al. Food & Function, 2010, 1:90-98; Simon et al. Molecular Nutrition and Food Research, 2009, 53:332-340; Spassieva and Bieberich Anticancer Agents in Medicinal Chemistry, 2011, 11:882-890; Tilly and Kolesnick Biochemical et Biophysica Acta, 2002, 1585:135-138) The biosynthetic/metabolic pathways of sphingolipids remain challenging to unravel, particularly due to the facile interconversion between different metabolic forms often exhibiting opposing function/activity. Ceramides, central to all sphingolipid metabolism, have been implicated in various antiproliferative responses. On the other hand, the presence of elevated levels of glucosylceramides has been linked to drug resistance and metastasis. Sphingolipid metabolites can thus be understood as important targets in understanding the signaling pathways leading to drug resistant and metastatic cancers.

The problems mentioned above regarding both intrinsic and acquired resistance to specific apoptotics remain a challenge to overcome. A prime example of this is the TNF-related apoptosis-inducing ligand (TRAIL). Since its discovery in 1995, TRAIL has been under intense study for its potential as a selective anticancer agent in cancer therapy because it preferentially induces apoptosis in tumor cells but not in normal cells (Wiley S R, et al. Immunity 1995; 3(6):673-82; Holoch P A and Griffith T S. European Journal of Pharmacology 2009; 625(1-3):63-72). However, the success of TRAIL-based cancer therapy so far is limited as cancer cells, especially metastatic cancer cells, often exhibit a TRAIL-resistance phenotype (Galligan L, et al. Molecular Cancer Therapies 2005; 4(12):2026-36; White-Gilbertson S, et al. Oncogene 2009; 28(8):1132-41; Garofalo M, et al. Cancer Cell 2009; 16(6):498-509; Kim S H, et al. Cancer Research 2008; 68(7):2062-4). Thus, there is a need for compositions for treating TRAIL resistant, or multi-drug resistant cancer cells.

Therefore, it is an object of the invention to provide synthetic compounds for treating drug resistant cancers and tumors.

It is a further object of the invention to provide methods for synthesizing new compounds for treating drug resistant cancers and tumors.

It is a still another object of the invention to provide methods and compositions for treating cancer and tumors, in particular drug resistant cancer and tumors.

It is yet another object of the invention to provide methods and compositions to sensitize cancer and tumor cells to existing treatments.

### SUMMARY OF THE INVENTION

Synthetic compounds that are mimetics of naturally occurring sphingolipid metabolites are provided. Although the mimetics structurally resemble naturally occurring sphingolipid metabolites, the disclosed mimetics are not naturally occurring sphingolipid metabolites. In some embodiments the sphingolipid metabolite mimetics have the general formula

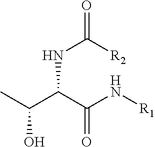

wherein R₁, R₂, and X are chosen appropriately and as described below.

Exemplary mimetics include, but are not limited to the compounds designated as NZJU1, NZJU2, NZJU4, NZJU10(NZJU2c), NZJU11(NZJU3c), NZJU12(NZJU4c), NZJU13(NZJU2b), NZJU14(NZJU3b), NZJU15(NZJU4b), NZJU16(NZJU2a), NZJU17(NZJU3a), NZJU18(NZJU4a), NZJU19, NZJU24(NZJU2d), NZJU25(NZJU3d), NZJU26(NZJU4d), NZJU27(NZJU2e), NZJU28(NZJU3 e), NZJU29(NZJU4e), NZJU30(NZJU2f), NZJU31(NZJU3f), NZJU32(NZJU4f), NZJU33(NZJU2g), NZJU34(NZJU3g), NZJU35(NZJU4g), NZJU36(NZJU2h), NZJU37(NZJU3h), NZJU38(NZJU4h), NZJU39(NZJU1a), NZJU40(NZJU1b), NZJU41(NZJU1c), NZJU42(NZJU1d), NZJU43(NZJU1f), NZJU44(NZJU1g), NZJU45(NZJU1h), NZJU46, NZJU47(NZJU2i), NZJU48(NZJU3i), NZJU49(NZJU4i), NZJU50(NZJU2j), NZJU51(NZJU3j), NZJU52(NZJU4j), NZJU53, NZJU54, NZJU55, NZJU56, NZJU57, NZJU58, NZJU59, NZJU60, NZJU61, NZJU62, NZJU63, and NZJU64, enantiomers thereof, derivatives thereof, or prodrugs thereof. In some instances the mimetics provided herein exhibit direct anti-cancer cell activity. Examples of these include, but are not limited to compounds designated herein as NZJU13(NZJU2b), NZJU16(NZJU2a), NZJU26(NZJU4d), NZJU27(NZJU2e), NZJU28(NZJU3e), NZJU29(NZJU4e), NZJU30(NZJU2f), NZJU33(NZJU2g), NZJU34(NZJU3g), NZJU36(NZJU2h), NZJU37(NZJU3h), NZJU39(NZJU1a), NZJU41(NZJU1c), NZJU44(NZJU1g), NZJU45(NZJU1h), NZJU46, NZJU47(NZJU2i), NZJU48(NZJU3i), NZJU51(NZJU3j), NZJU57, NZJU60, and NZJU62. Preferably the mimetics provided herein sensitize cancer cells to TRAIL-induced apoptosis. Examples of mimetics exhibiting TRAIL sensitization activity include, but are not limited to NZJU13(NZJU2b), NZJU16(NZJU2a), NZJU30(NZJU2f), NZJU34(NZJU3g), NZJU41(NZJU1c), NZJU44(NZJU1g), and NZJU45(NZJU1h). All of these compounds are described more fully below.

Methods for synthesizing the disclosed mimetics and intermediates useful for the synthesis of sphingolipid metabolite mimetics are also provided. In some embodiments the intermediates have the general formula

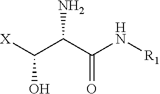

wherein R₁ and X are chosen appropriately as described below. Certain intermediates can also act as sphingolipid metabolite mimetics.

The disclosed sphingolipid metabolite mimetics are useful as anti-tumor agents for cancer or tumor treatment. Representative formulations and methods for treating cancer contain an effective amount of one or more sphingolipid metabolite mimetics to reduce tumor burden, promote tumor regression, or treat a symptom of cancer. In other embodiments the sphingolipid metabolite mimetic is formulated with one or more additional sphingolipid metabolite mimetics, with one or more other chemotherapeutic agents, with one or more anti-cancer agents, for example TRAIL, or combinations thereof. The sphingolipid metabolite mimetics can be in an effective amount to sensitize cells to TRAIL-induced apoptosis in combination with an effective amount of a death receptor agonist. For example, the death receptor agonist can be TRAIL or an antibody that selectively binds and activates DR4 (TRAIL-R1) or DR5 (TRAIL-R2).

Methods for treating drug resistant cancers or tumors are also provided. These methods include administering to the subject a composition containing a therapeutically effective amount of one or more of the disclosed sphingolipid metabolite mimetics. The sphingolipid metabolite mimetic composition can be administered to a subject in combination or alternation with a second therapeutic composition, for example a death receptor agonist. Other representative methods involve administering to the subject a first composition containing a therapeutically effective amount of one or more sphingolipid metabolite mimetics and a second composition containing a therapeutically effective amount of a death receptor agonist. For example, the first composition can be administered from about 1 minute to about 1 hour before the second composition. Alternatively, the first composition can be administered less than minute before the second composition. In preferred embodiments, the cancer cell is resistant to TNF-related apoptosis-inducing ligand (TRAIL) treatment.

### BRIEF DESCRIPTION OF THE DRAWINGS

FIG. 1 depicts the role that ceramides play in the generation of and the interconversion between the indicated sphingolipid metabolites.

FIG. 2 is a bar graph of % tumor cell growth inhibition determined from MTT assays with HepG2, A431, SA, MCF-7, and SiHa cell lines for NZJU4. The specific tumor cell lines are indicated under the x axis, and the % tumor cell growth inhibition is indicated by the y axis.

FIG. 3 is a bar graph of % tumor cell growth inhibition determined from MTT assays with HepG2, A431, SA, MCF-7, and SiHa cell lines for NZJU15(NZJU4b). The specific tumor cell lines are indicated under the x axis, and the % tumor cell growth inhibition is indicated by the y axis.

FIG. 4 is a bar graph of % tumor cell growth inhibition determined from MTT assays with HepG2, A431, SA, MCF-7, and SiHa cell lines for NZJU27 (NZJU3h). The specific tumor cell lines are indicated under the x axis, and the % tumor cell growth inhibition is indicated by the y axis.

FIG. 5 is a bar graph of % tumor cell growth inhibition determined from MTT assays with HepG2, A431, SA, MCF-7, and SiHa cell lines for NZJU28. The specific tumor cell lines are indicated under the x axis, and the % tumor cell growth inhibition is indicated by the y axis.

FIG. 6 is a bar graph of % tumor cell growth inhibition determined from MTT assays with HepG2, A431, SA, MCF-7, and SiHa cell lines for NZJU31 The specific tumor cell lines are indicated under the x axis, and the % tumor cell growth inhibition is indicated by the y axis.

FIG. 7 is a bar graph of % tumor cell growth inhibition determined from MTT assays with HepG2, A431, SA, MCF-7, and SiHa cell lines for NZJU32. The specific tumor cell lines are indicated under the x axis, and the % tumor cell growth inhibition is indicated by the y axis.

FIG. 8 is a bar graph of % tumor cell growth inhibition determined from MTT assays with HepG2, A431, SA, MCF-7, and SiHa cell lines for NZJU33. The specific tumor cell lines are indicated under the x axis, and the % tumor cell growth inhibition is indicated by the y axis.

FIG. 9 is a bar graph of % tumor cell growth inhibition determined from MTT assays with HepG2, A431, SA, MCF-7, and SiHa cell lines for NZJU36. The specific tumor cell lines are indicated under the x axis, and the % tumor cell growth inhibition is indicated by the y axis.

FIG. 10 is a bar graph of % tumor cell growth inhibition determined from MTT assays with HepG2, A431, SA, MCF-7, and SiHa cell lines for NZJU37. The specific tumor cell lines are indicated under the x axis, and the % tumor cell growth inhibition is indicated by the y axis.

FIG. 11 is a bar graph of % tumor cell growth inhibition determined from MTT assays with HepG2, A431, SA, MCF-7, and SiHa cell lines for NZJU42. The specific tumor cell lines are indicated under the x axis, and the % tumor cell growth inhibition is indicated by the y axis.

FIG. 12 is a bar graph of % tumor cell growth inhibition determined from MTT assays with HepG2, A431, SA, MCF-7, and SiHa cell lines for NZJU43. The specific tumor cell lines are indicated under the x axis, and the % tumor cell growth inhibition is indicated by the y axis.

FIG. 13 is a bar graph of % tumor cell growth inhibition determined from MTT assays with A431, SA, MCF-7, and SiHa cell lines for NZJU46. The specific tumor cell lines are indicated under the x axis, and the % tumor cell growth inhibition is indicated by the y axis.

FIG. 14 is a bar graph of % tumor cell growth inhibition determined from MTT assays with HepG2, A431, SA, SiHa, SW480, and SW620 cell lines for NZJU47. The specific tumor cell lines are indicated under the x axis, and the % tumor cell growth inhibition is indicated by the y axis.

FIG. 15 is a bar graph of % tumor cell growth inhibition determined from MTT assays with HepG2, A431, SA, SiHa, SW480, and SW620 cell lines for NZJU48. The specific tumor cell lines are indicated under the x axis, and the % tumor cell growth inhibition is indicated by the y axis.

FIG. 16 is a bar graph of % tumor cell growth inhibition determined from MTT assays with HepG2, A431, SA, SiHa, SW480, and SW620 cell lines for NZJU49. The specific tumor cell lines are indicated under the x axis, and the % tumor cell growth inhibition is indicated by the y axis.

FIG. 17 is a bar graph of % tumor cell growth inhibition determined from MTT assays with HepG2, A431, SA, SiHa, SW480, and SW620 cell lines for NZJU50. The specific tumor cell lines are indicated under the x axis, and the % tumor cell growth inhibition is indicated by the y axis.

FIG. 18 is a bar graph of % tumor cell growth inhibition determined from MTT assays with HepG2, A431, SA, SiHa, SW480, and SW620 cell lines for NZJU51. The specific tumor cell lines are indicated under the x axis, and the % tumor cell growth inhibition is indicated by the y axis.

FIG. 19 is a bar graph of % tumor cell growth inhibition determined from MTT assays with HepG2, A431, SA, SiHa, SW480, and SW620 cell lines for NZJU52. The specific tumor cell lines are indicated under the x axis, and the % tumor cell growth inhibition is indicated by the y axis.

FIG. 20 is a bar graph of % tumor cell growth inhibition determined from MTT assays with HepG2, A431, SA, SiHa, SW480, and SW620 cell lines for NZJU49. The specific tumor cell lines are indicated under the x axis, and the % tumor cell growth inhibition is indicated by the y axis.

FIG. 21 is a bar graph of % tumor cell growth inhibition determined from MTT assays with HepG2, A431, SA, SiHa, SW480, and SW620 cell lines for NZJU54. The specific tumor cell lines are indicated under the x axis, and the % tumor cell growth inhibition is indicated by the y axis.

FIG. 22 is a bar graph of % tumor cell growth inhibition determined from MTT assays with HepG2, A431, SA, SiHa, SW480, and SW620 cell lines for NZJU55. The specific tumor cell lines are indicated under the x axis, and the % tumor cell growth inhibition is indicated by the y axis.

FIG. 23 is a bar graph of % tumor cell growth inhibition determined from MTT assays with HepG2, A431, SA, SiHa, SW480, and SW620 cell lines for NZJU56. The specific tumor cell lines are indicated under the x axis, and the % tumor cell growth inhibition is indicated by the y axis.

FIG. 24 is a bar graph of % tumor cell growth inhibition determined from MTT assays with HepG2, A431, SA, SiHa, SW480, and SW620 cell lines for NZJU57. The specific tumor cell lines are indicated under the x axis, and the % tumor cell growth inhibition is indicated by the y axis.

FIG. 25 is a bar graph of % tumor cell growth inhibition determined from MTT assays with HepG2, A431, SA, SiHa, SW480, and SW620 cell lines for NZJU58. The specific tumor cell lines are indicated under the x axis, and the % tumor cell growth inhibition is indicated by the y axis.

FIG. 26 is a bar graph of % tumor cell growth inhibition determined from MTT assays with HepG2, A431, SA, SiHa, SW480, and SW620 cell lines for NZJU59. The specific tumor cell lines are indicated under the x axis, and the % tumor cell growth inhibition is indicated by the y axis.

FIG. 27 is a bar graph of % tumor cell growth inhibition determined from MTT assays with HepG2, A431, SA, SiHa, SW480, and SW620 cell lines for NZJU60. The specific tumor cell lines are indicated under the x axis, and the % tumor cell growth inhibition is indicated by the y axis.

FIG. 28 is a bar graph of % tumor cell growth inhibition determined from MTT assays with HepG2, A431, SA, SiHa, SW480, and SW620 cell lines for NZJU63. The specific tumor cell lines are indicated under the x axis, and the % tumor cell growth inhibition is indicated by the y axis.

FIG. 29 is a bar graph of % tumor cell growth inhibition determined from MTT assays with HepG2, A431, SA, SiHa, SW480, and SW620 cell lines for NZJU64. The specific tumor cell lines are indicated under the x axis, and the % tumor cell growth inhibition is indicated by the y axis.

FIG. 30 includes fluorescent micrographs showing apoptotic cell death after sphingolipid mimetics treatment with NZJU27 (panel B), NZJU28 (panel C), NZJU36 (panel D), NZJU37 (panel E), NZJU48 (panel F), and NZJU51 (panel G). DMSO (panel A) is used as a control.

FIG. 31 is a bar graph of the cell sensitivity measured as % growth inhibition after exposure to the sphingolipid metabolite mimetics NZJU13, NZJU16, NZJU30, NZJU34, NZJU41, NZJU44, and NZJU45 alone or in combination with TRAIL as determined by MTT assays with the SW620 cell line. The bars depict the % growth inhibition to the sphingolipid metabolite mimetic when administered alone or in combination with TRAIL as well as the enhancement due to the co-administration. A bar (far left) depicting the % growth inhibition for TRAIL exposure alone is provided as a reference.

FIG. 32 is a bar graph of the cell sensitivity measured as % growth inhibition after exposure to the sphingolipid metabolite mimetics NZJU13, NZJU16, NZJU30, NZJU34, NZJU41, NZJU44, and NZJU45 alone or in combination with TRAIL as determined by MTT assays with the MCF-7 cell line. The bars depict the % growth inhibition to the sphingolipid metabolite mimetic when administered alone or in combination with TRAIL as well as the enhancement due to the co-administration. A bar (far left) depicting the % growth inhibition for TRAIL exposure alone is provided as a reference.

FIG. 33 is a bar graph of the cell sensitivity measured as % growth inhibition after exposure to the sphingolipid metabolite mimetics NZJU13, NZJU16, NZJU30, NZJU34, NZJU41, NZJU44, and NZJU45 alone or in combination with TRAIL as determined by MTT assays with the HeLa cell line. The bars depict the % growth inhibition to the sphingolipid metabolite mimetic when administered alone or in combination with TRAIL as well as the enhancement due to the co-administration. A bar (far left) depicting the % growth inhibition for TRAIL exposure alone is provided as a reference.

FIG. 34 is a bar graph of the cell sensitivity measured as % growth inhibition after exposure to the sphingolipid metabolite mimetics NZJU13, NZJU16, NZJU30, NZJU34, NZJU41, NZJU44, and NZJU45 alone or in combination with TRAIL as determined by MTT assays with the HepG2 cell line. The bars depict the % growth inhibition to the sphingolipid metabolite mimetic when administered alone or in combination with TRAIL as well as the enhancement due to the co-administration. A bar (far left) depicting the % growth inhibition for TRAIL exposure alone is provided as a reference.

FIG. 35 is a bar graph of the cell sensitivity measured as % growth inhibition after exposure to the sphingolipid metabolite mimetics NZJU13, NZJU16, NZJU30, NZJU34, NZJU41, NZJU44, and NZJU45 alone or in combination with TRAIL as determined by MTT assays with the LS411N cell line. The bars depict the % growth inhibition to the sphingolipid metabolite mimetic when administered alone or in combination with TRAIL as well as the enhancement due to the co-administration. A bar (far left) depicting the % growth inhibition for TRAIL exposure alone is provided as a reference.

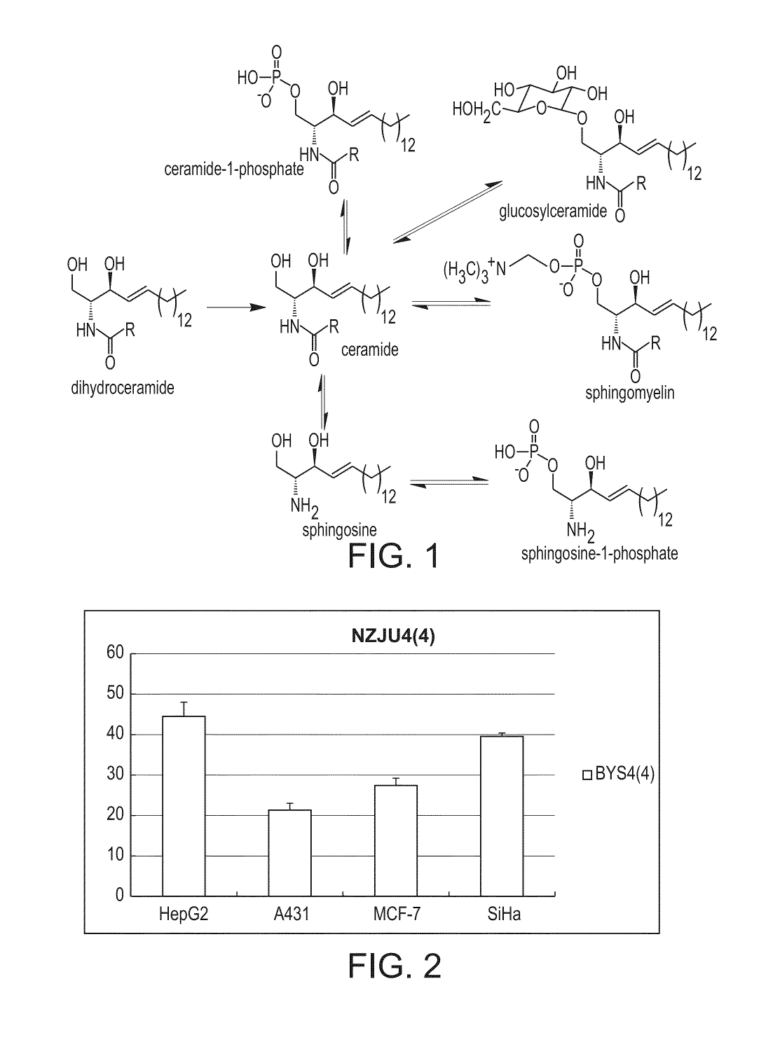

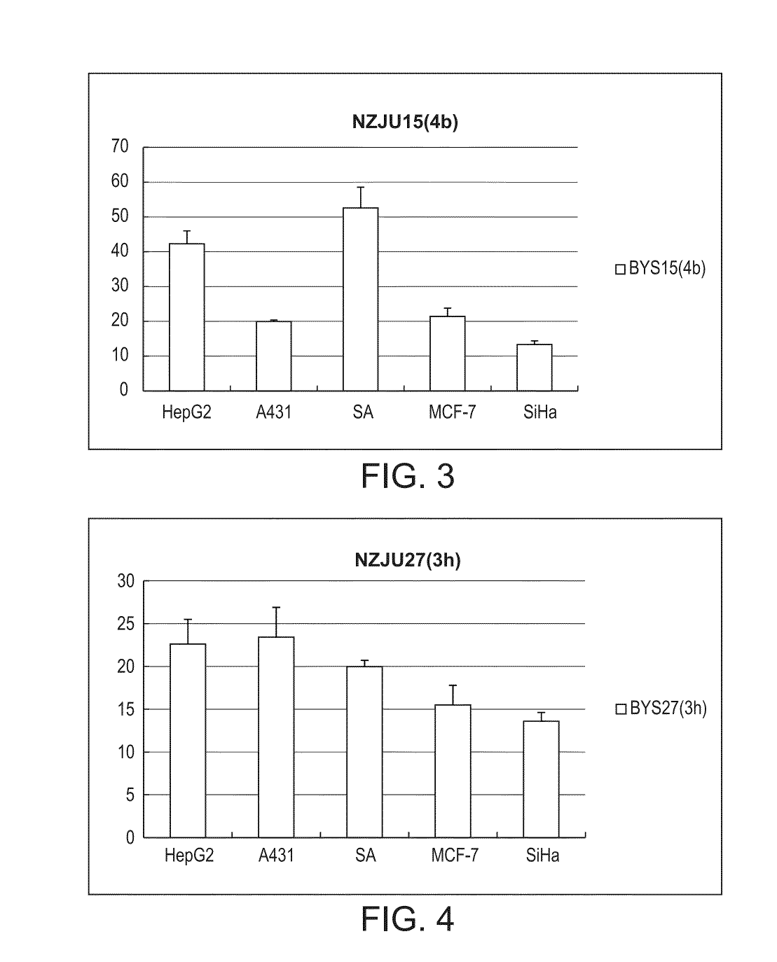

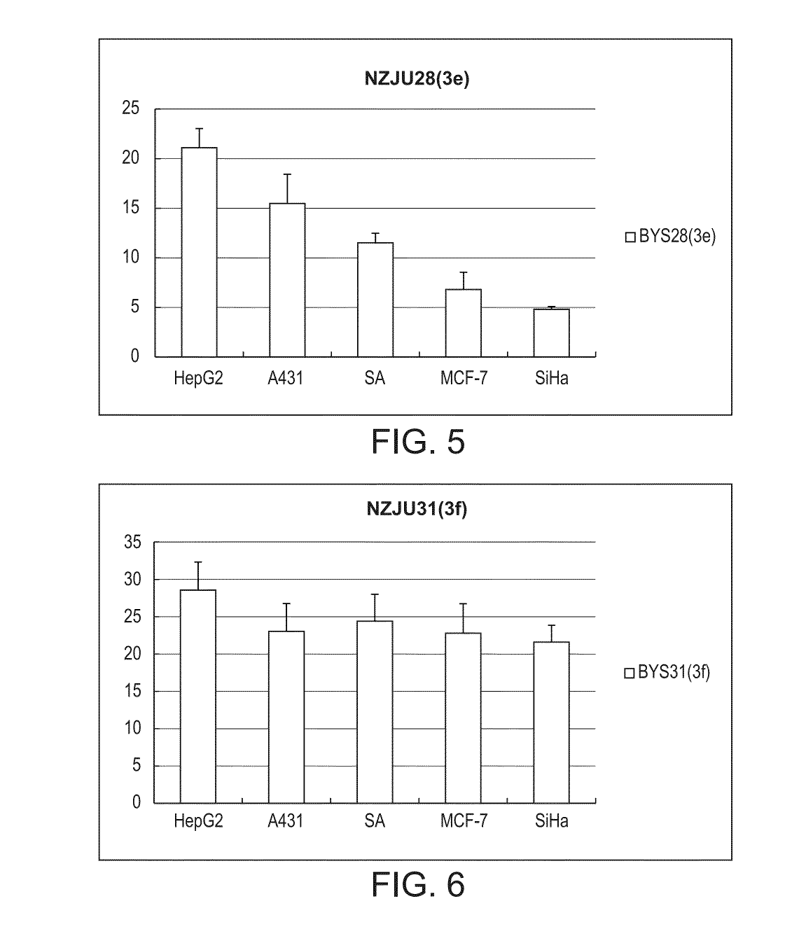

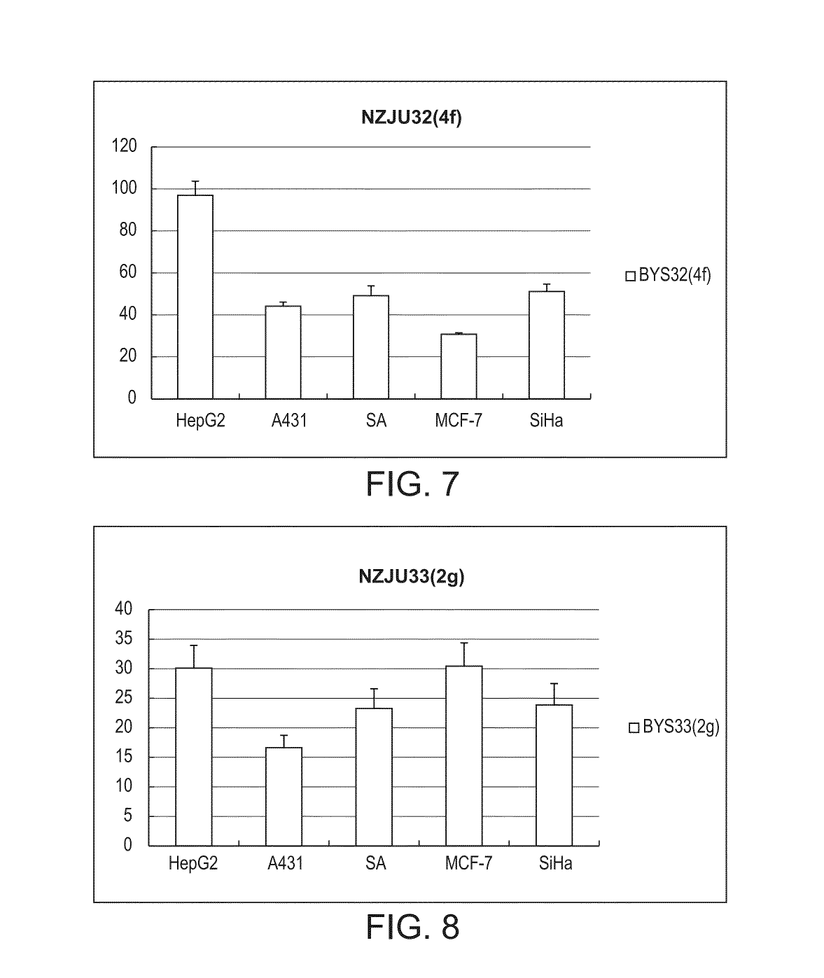

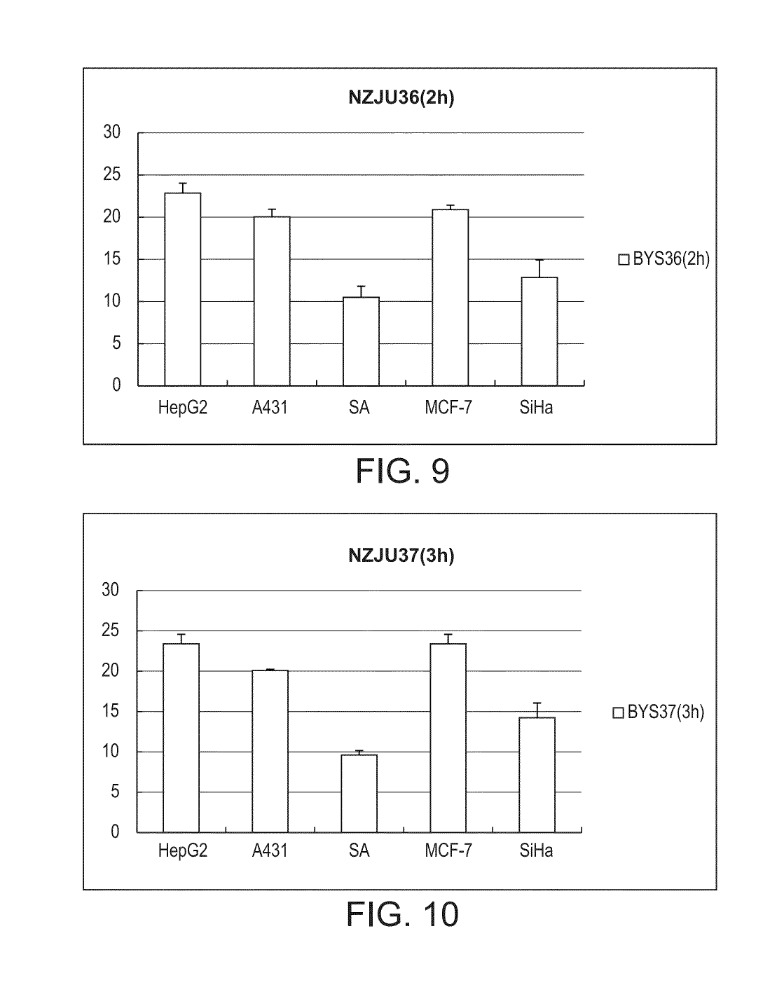

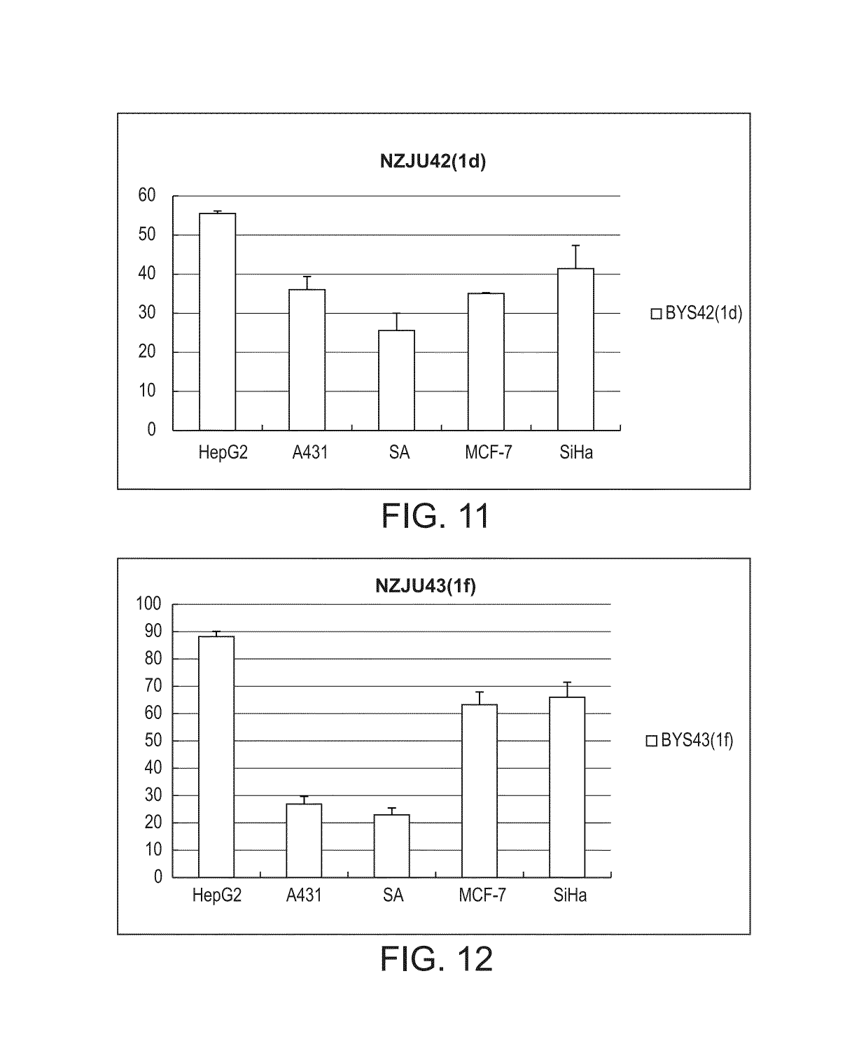

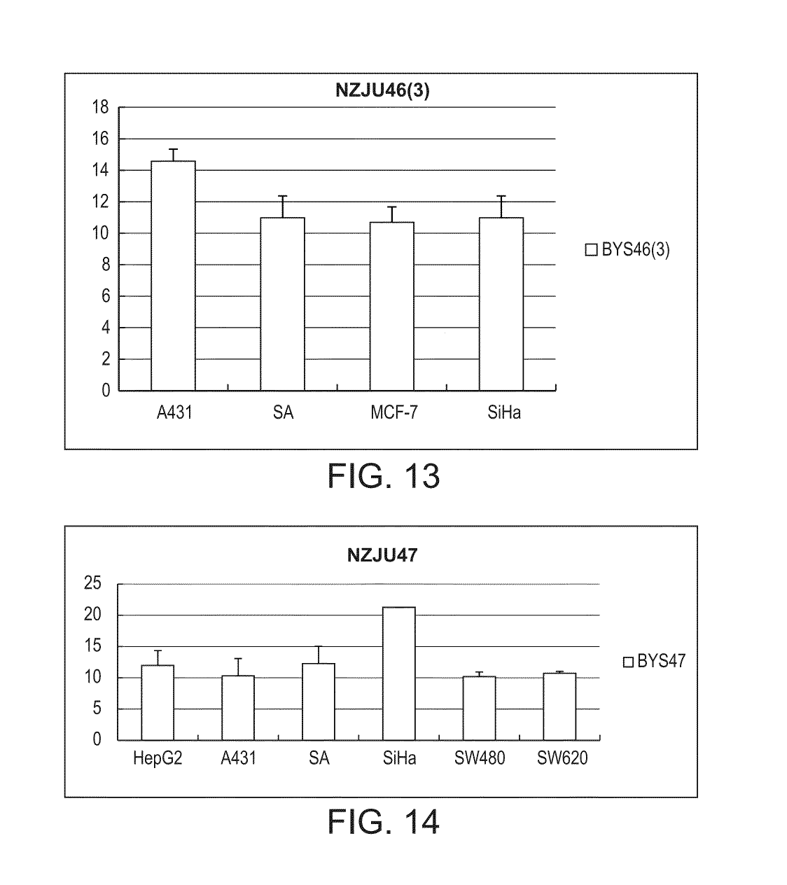

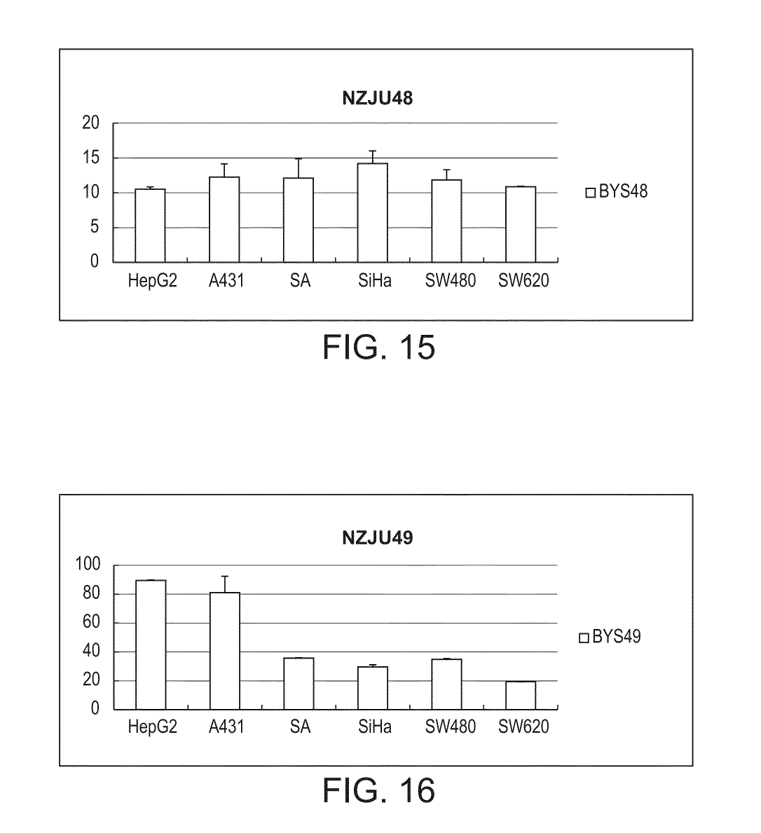

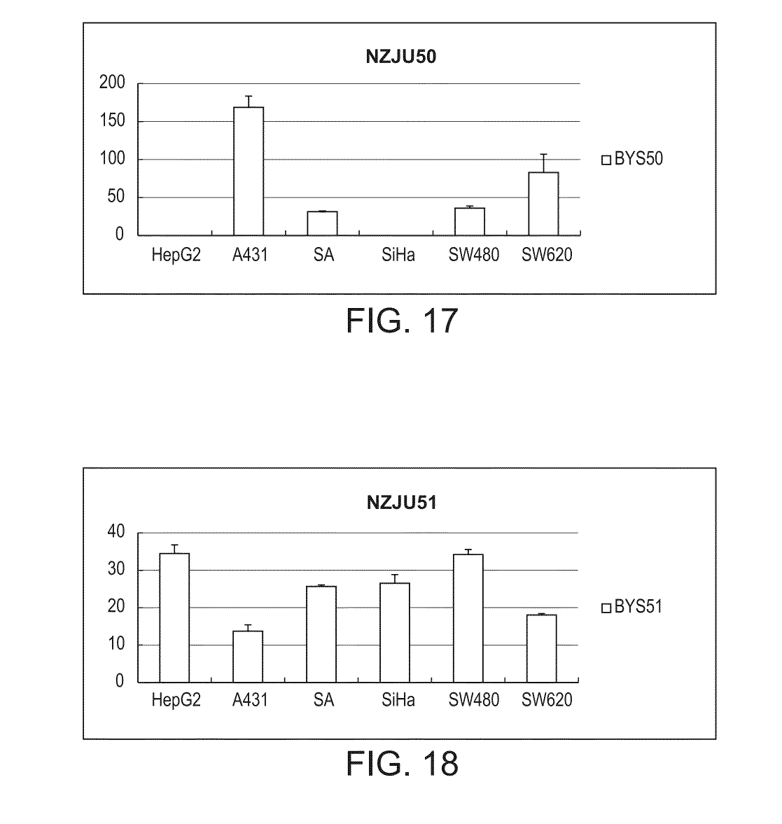

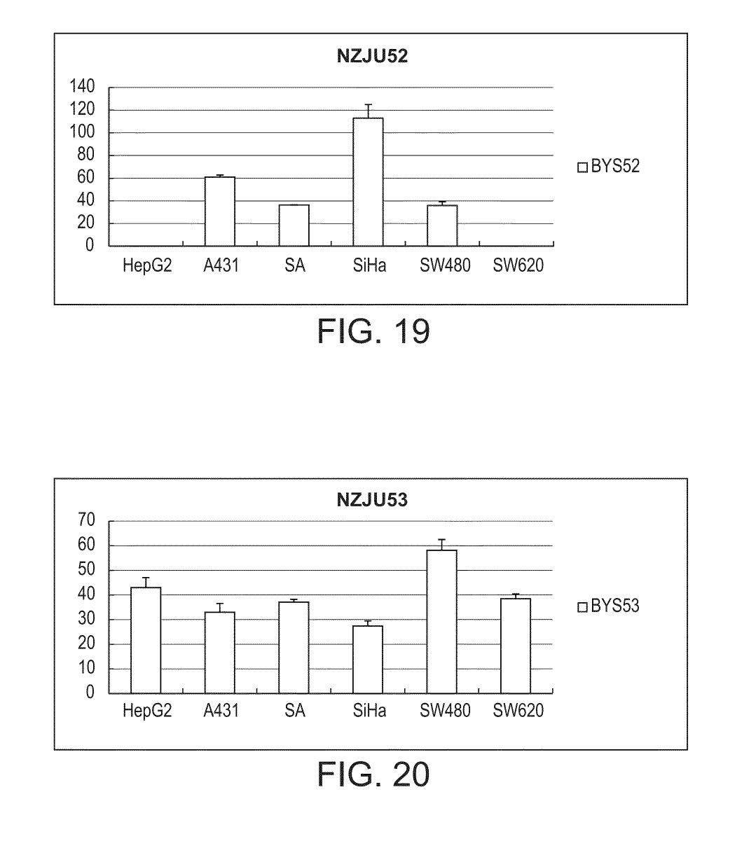

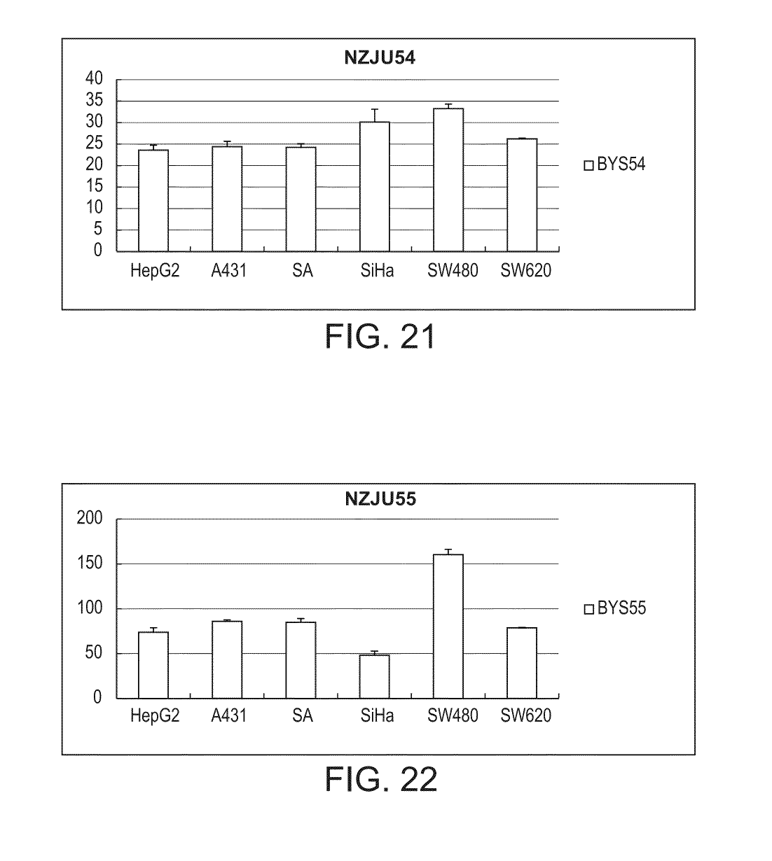

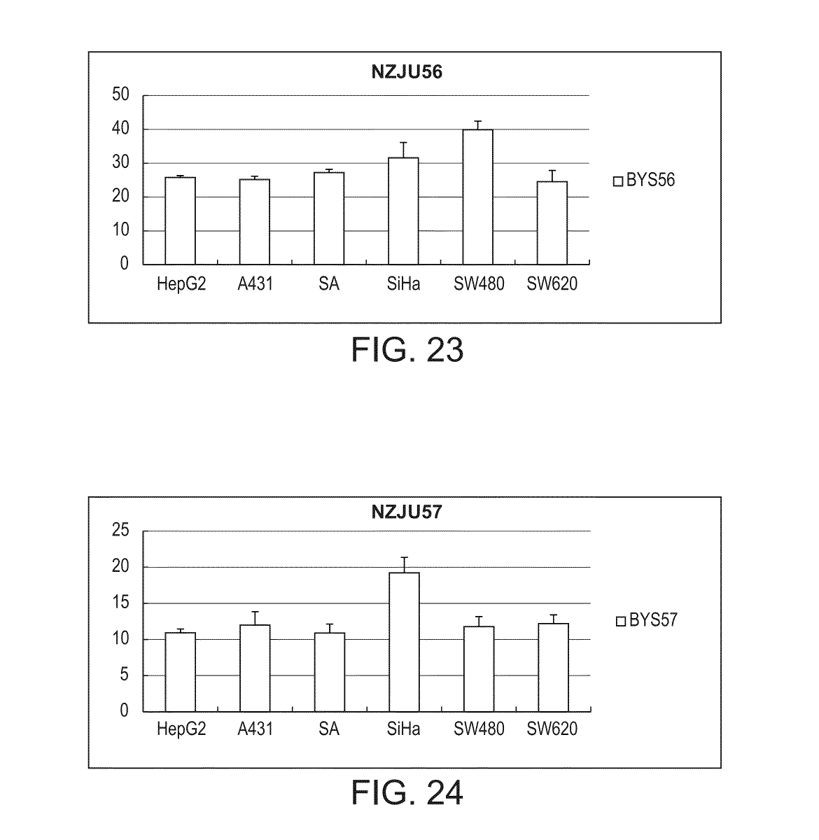

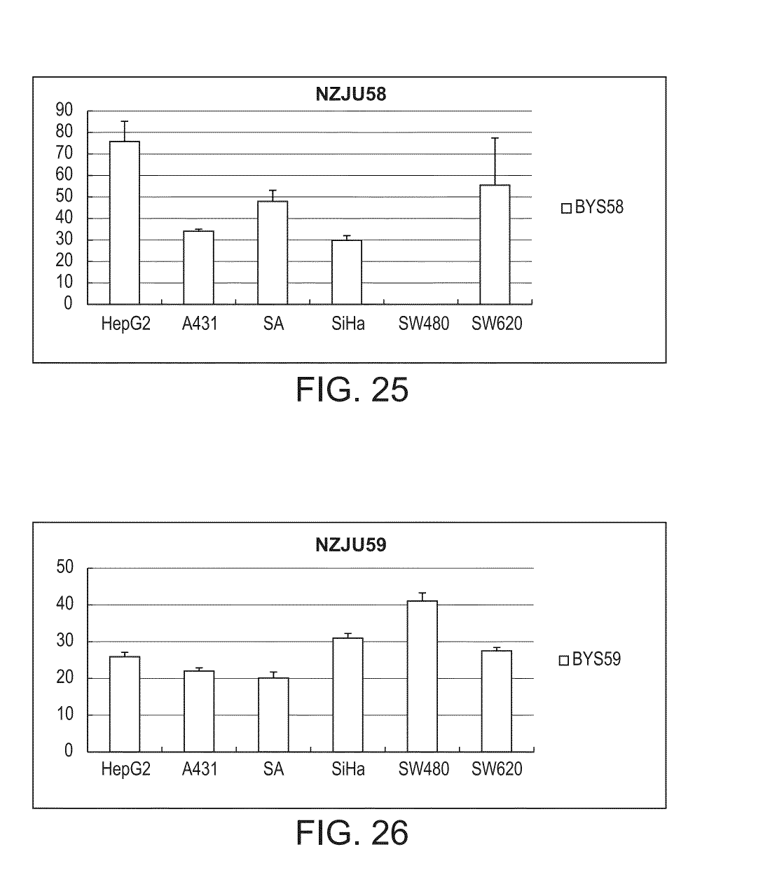

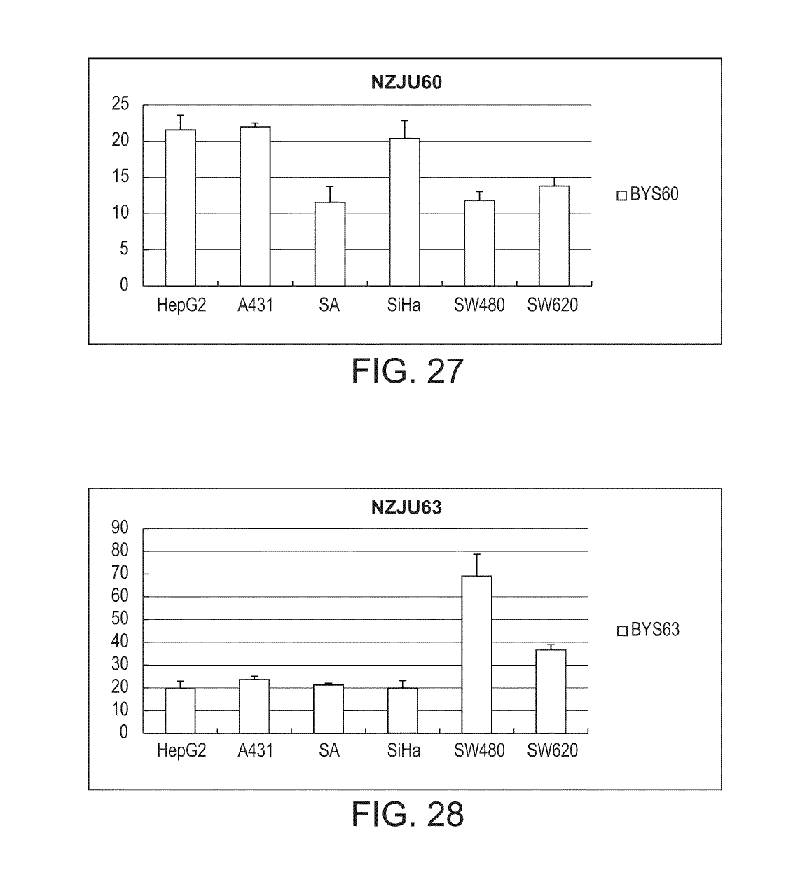

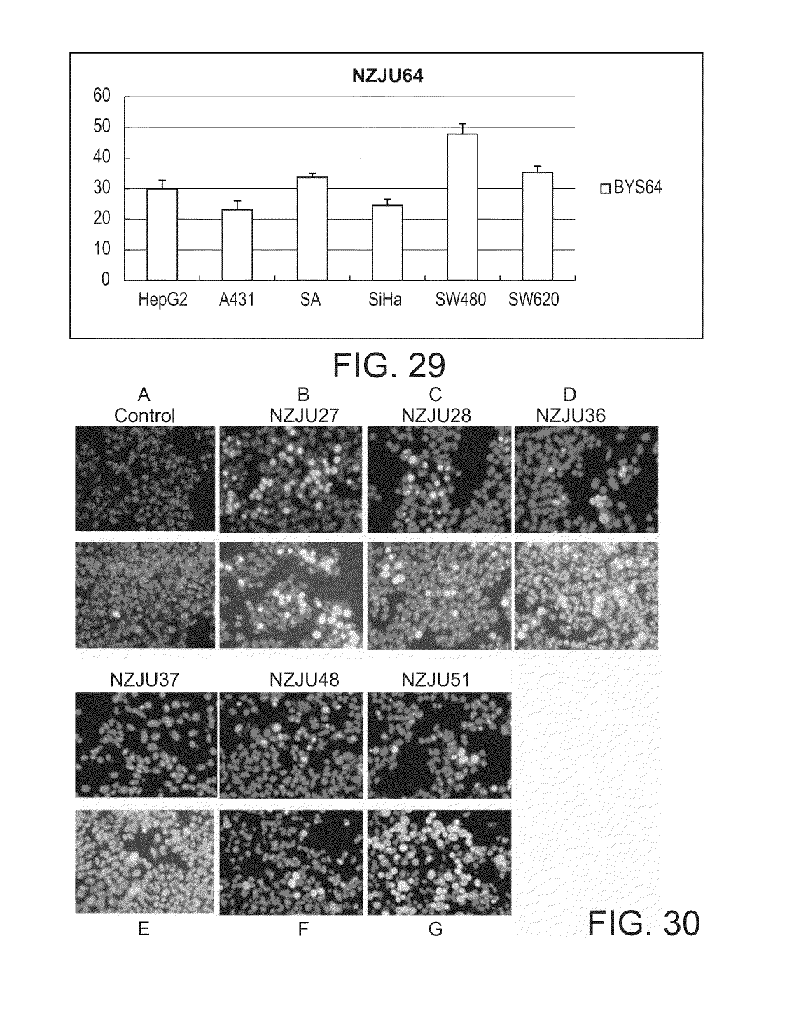

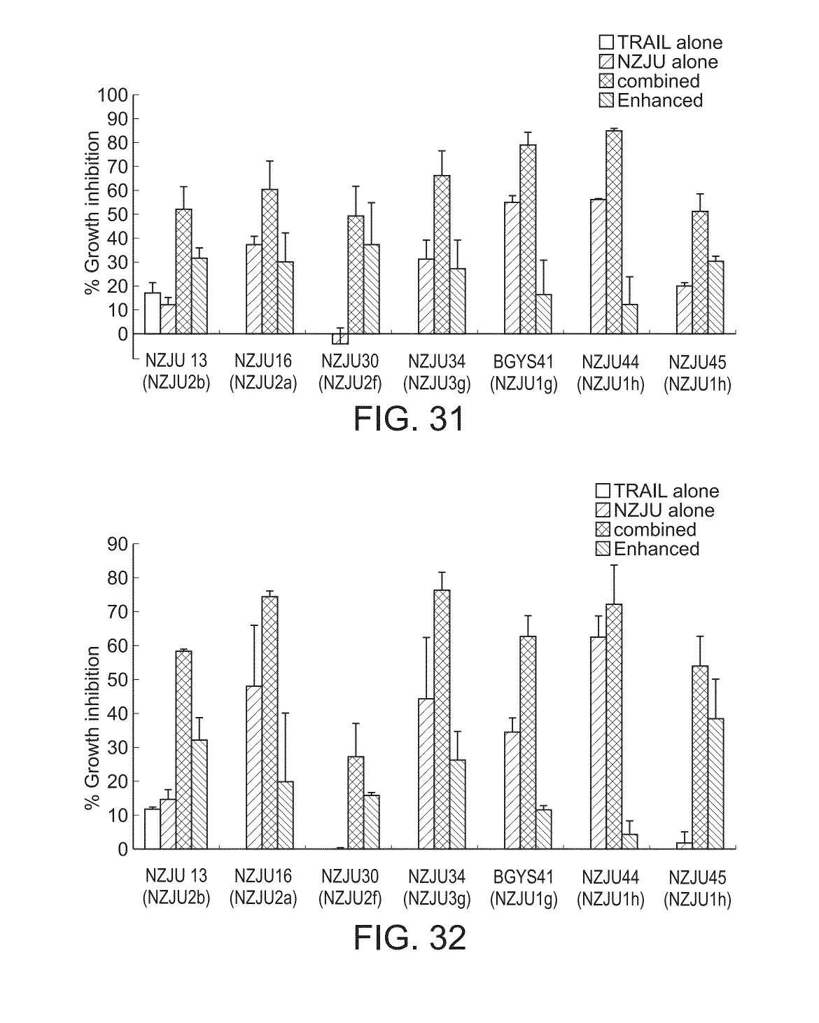

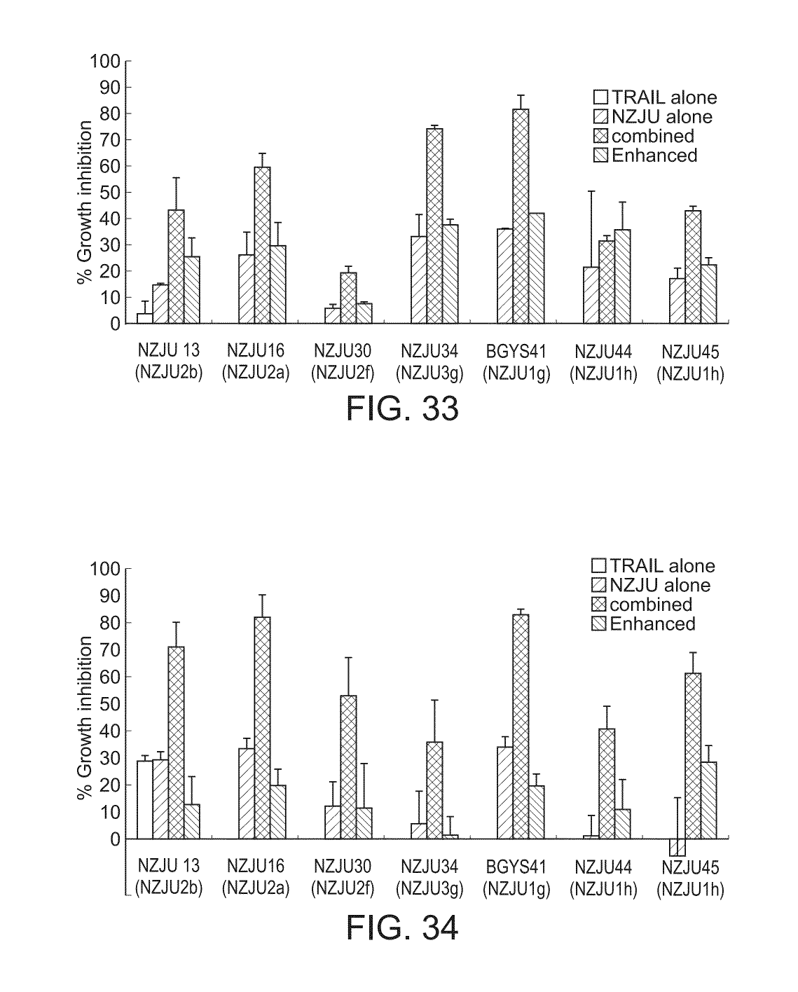

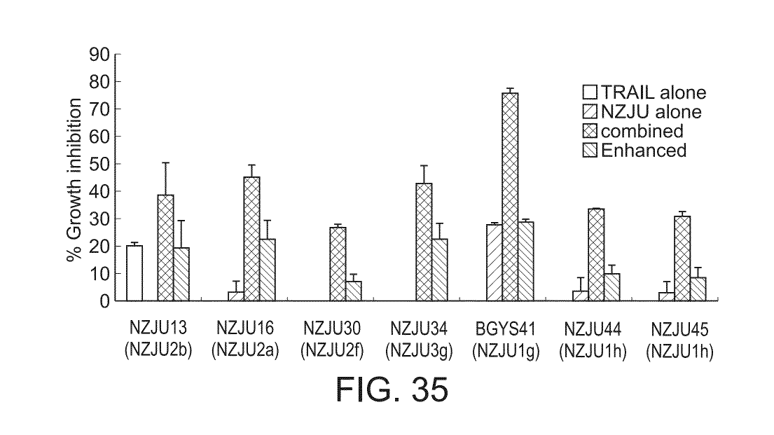

### DETAILED DESCRIPTION OF THE INVENTION

### I. Sphingolipid Metabolite Mimetics

A variety of sphingolipid metabolite mimetics are provided. Sphingolipid metabolite mimetics may be synthetically derived or may be isolated from natural sources using methods known to those of skill in the art. In certain preferred embodiments the sphingolipid metabolite mimetics are not naturally occurring metabolites.

Synthetic sphingolipid metabolite mimetics have been generated both i) to explore the complicated and interconnected signaling pathways of these metabolites and ii) as potential therapeutics for proliferative disorders such as cancer. Importantly, the biological activities of sphingolipids and sphingolipid metabolites demonstrate large variations with small changes in the overall molecular structure (Brockman, H. L. et al., Drug Design Reviews—Online 87:1722-1731, 2004). The C₄-C₅ double bond is thought to be necessary for apoptotic activity as in nearly all studies the dihydroceramide is found to be inactive (Radin, N. S., Bioorganic and Medicinal Chemistry 11:2133-2142, 2003).

A. Exemplary Sphingolipid Metabolite Mimetics

An exemplary sphingolipid metabolite mimetic is represented by the general formula:

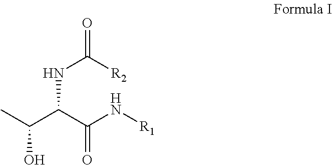

wherein R₁, R₂, and X taken independently may be a hydrogen atom, a halogen atom, a hydroxyl group, or any other organic groupings containing any number of carbon atoms and optionally including one or more heteroatoms such as oxygen, sulfur, or nitrogen grouping in linear, branched, or cyclic structural formats, representative R₁, R₂, and X groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group.

In some instances the sphingolipid metabolite mimetic is represented by Formula I wherein X is selected from the group consisting of hydrogen, methyl, alkyl, or substituted alkyl; R₁ is alkyl or substituted alkyl, preferably a linear alkyl, more preferably a C₁₀-C₂₀ alkyl or linear alkyl; and R₂ is selected from the group consisting of alkenyl, substituted alkenyl, aryl, or heteroaryl.

In certain preferred embodiments the sphingolipid metabolite mimetic is represented by Formula I wherein X is methyl or hydrogen, preferably methyl; R₁ is linear alkyl, preferably a linear C₁₀-C₂₀ alkyl; and R₂ is linear alkenyl or aryl, preferably a linear C₃-C₇ alkenyl or an aryl or heteroaryl containing 1 or 2 fused 5-membered or 6-membered rings.

In some embodiments the sphingolipid metabolite mimetic is represented by Formula I wherein X is selected from the group consisting of hydrogen, methyl, alkyl, or substituted alkyl; R₁ is alkyl or substituted alkyl, preferably a linear alkyl, more preferably a C₁₀-C₂₀ alkyl; and R₂ is selected from the group consisting of phenyl, 2-furanyl, 2-thiophenyl, 2,4-heaxdienyl, 3-phenyl acrylic, isoquinoline, indole, methyl indole, benzofuranyl, cyclohex-4-ene-1,2,3-triol, naphthalene, and toluene.

Other sphingolipid metabolite mimetics are represented by the general formula:

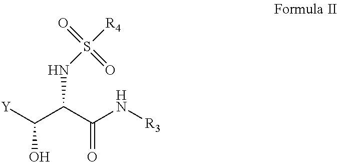

wherein R₃, R₄, and Y taken independently may be a hydrogen atom, a halogen atom, a hydroxyl group, or any other organic groupings containing any number of carbon atoms and optionally including one or more heteroatoms such as oxygen, sulfur, or nitrogen grouping in linear, branched, or cyclic structural formats, representative R₃, R₄, and Y groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group.

Still other sphingolipid metabolite mimetics are represented by Formula II wherein Y is selected from the group consisting of hydrogen, methyl, alkyl, or substituted alkyl; R₃ is alkyl or substituted alkyl, preferably a linear alkyl, more preferably a C₁₀-C₂₀ alkyl or linear alkyl; and R₄ is selected from the group consisting of alkenyl, substituted alkenyl, aryl, or heteroaryl.

In other embodiments the sphingolipid metabolite mimetic is represented by Formula II wherein Y is methyl or hydrogen, preferably methyl; R₃ is linear alkyl, preferably a linear C₁₀-C₂₀ alkyl; and R₄ is linear alkenyl or aryl, preferably a linear C₃-C₇ alkenyl or an aryl or heteroaryl containing 1 or 2 fused 5-membered or 6-membered rings.

Additional sphingolipid metabolite mimetics are represented by Formula II wherein Y is selected from the group consisting of hydrogen, methyl, alkyl, or substituted alkyl; R₃ is alkyl or substituted alkyl, preferably a linear alkyl, more preferably a C₁₀-C₂₀ alkyl; and R₄ is selected from the group consisting of phenyl, 2-furanyl, 2-thiophenyl, 2,4-heaxdienyl, 3-phenyl acrylic, isoquinoline, indole, methyl indole, benzofuranyl, cyclohex-4-ene-1,2,3-triol, naphthalene, and toluene.

A specific sphingolipid metabolite mimetic is (S)-2-Amino-3-hydroxy-N-dodecylpropanamide, the structure of which is shown below.

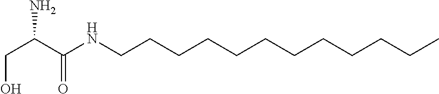 [SMILES: CCCCCCCCCCCCNC(=O)[C@@H](N)CO]

Another sphingolipid metabolite mimetic is (2S,3R)-2-Amino-3-hydroxy-N-dodecylbutanamide (herein referred to as NZJU2), the structure of which is shown below.

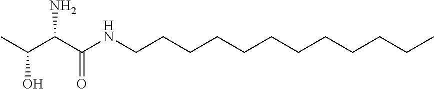 [SMILES: CCCCCCCCCCCCNC(=O)[C@@H](N)[C@@H](C)O]

Still another sphingolipid metabolite mimetic is (2S,3R)-2-Amino-3-hydroxy-N-octadecylbutanamide (herein referred to as NZJU4), the structure of which is shown below.

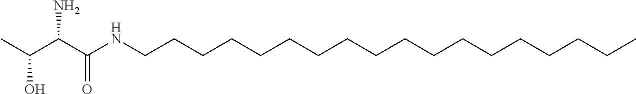 [SMILES: CCCCCCCCCCCCCCCCCCNC(=O)[C@@H](N)[C@@H](C)O]

Another sphingolipid metabolite mimetic is (2E,4E)-N-((2S,3R)-3-hydroxy-1-oxo-1-(dodecylamino)butan-2-yl)-2,4-hexadienoic acid amide (herein referred to as NZJU10 or NZJU2c), the structure of which is shown below.

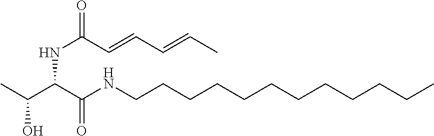 [SMILES: C/C=C/C=C/C(=O)N[C@H](C(=O)NCCCCCCCCCCCC)[C@@H](C)O]

Another sphingolipid metabolite mimetic is (2E,4E)-N-((2S,3R)-3-hydroxy-1-oxo-1-(tetradecylamino)butan-2-yl)-2,4-hexadienoic acid amide (herein referred to as NZJU11 or NZJU3c), the structure of which is shown below.

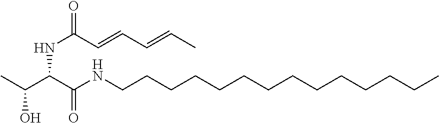 [SMILES: C/C=C/C=C/C(=O)N[C@H](C(=O)NCCCCCCCCCCCCCC)[C@@H](C)O]

Another sphingolipid metabolite mimetic is (2E,4E)-N-((2S,3R)-3-hydroxy-1-oxo-1-(octadecylamino)butan-2-yl)-2,4-hexadienoic acid amide (herein referred to as NZJU12 or NZJU4c), the structure of which is shown below.

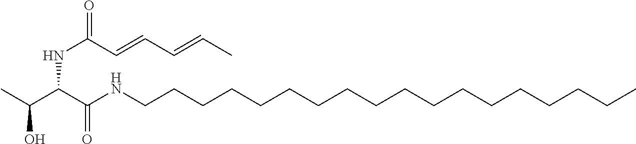 [SMILES: C/C=C/C=C/C(=O)N[C@H](C(=O)NCCCCCCCCCCCCCCCCCC)[C@H](C)O]

Another sphingolipid metabolite mimetic is N-((2S,3R)-3-hydroxy-1-oxo-1-(dodecylamino)butan-2-yl)-Furan-2-carboxamide (herein referred to as NZJU13 or NZJU2b), the structure of which is shown below.

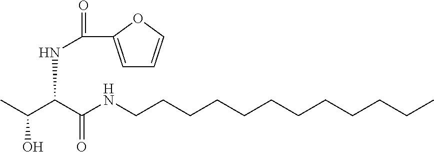 [SMILES: CCCCCCCCCCCCNC(=O)[C@@H](NC(=O)c1ccco1)[C@@H](C)O]

Another sphingolipid metabolite mimetic is N-((2S,3R)-3-hydroxy-1-oxo-1-(tetradecylamino)butan-2-yl)-Furan-2-carboxamide (herein referred to as NZJU14 or NZJU3b), the structure of which is shown below.

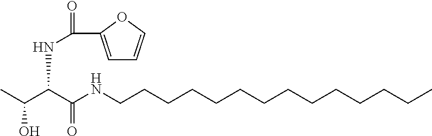 [SMILES: CCCCCCCCCCCCCCNC(=O)[C@@H](NC(=O)c1ccco1)[C@@H](C)O]

Another sphingolipid metabolite mimetic is N-((2S,3R)-3-hydroxy-1-oxo-1-(octadecylamino)butan-2-yl)-Furan-2-carboxamide (herein referred to as NZJU15 or NZJU4b), the structure of which is shown below.

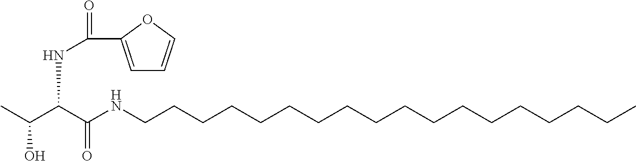 [SMILES: CCCCCCCCCCCCCCCCCCNC(=O)[C@@H](NC(=O)c1ccco1)[C@@H](C)O]

Another sphingolipid metabolite mimetic is N-((2S,3R)-3-hydroxy-1-oxo-1-(dodecylamino)butan-2-yl)-benzamide (herein referred to as NZJU16 or NZJU2a), the structure of which is shown below.

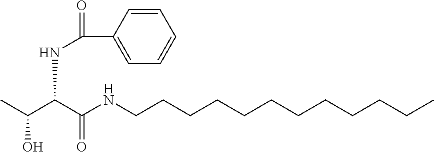 [SMILES: CCCCCCCCCCCCNC(=O)[C@@H](NC(=O)c1ccccc1)[C@@H](C)O]

Another sphingolipid metabolite mimetic is N-((2S,3R)-3-hydroxy-1-oxo-1-(tetradecylamino)butan-2-yl)-benzamide (herein referred to as NZJU17 or NZJU3a), the structure of which is shown below.

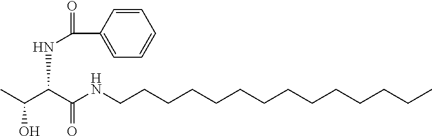 [SMILES: CCCCCCCCCCCCCCNC(=O)[C@@H](NC(=O)c1ccccc1)[C@@H](C)O]

Another sphingolipid metabolite mimetic is N-((2S,3R)-3-hydroxy-1-oxo-1-(octadecylamino)butan-2-yl)-benzamide (herein referred to as NZJU18 or NZJU4a), the structure of which is shown below.

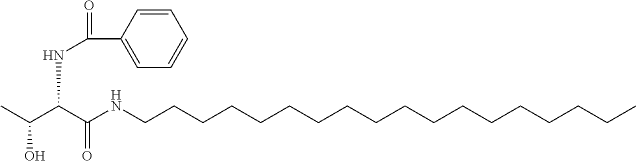 [SMILES: CCCCCCCCCCCCCCCCCCNC(=O)[C@@H](NC(=O)c1ccccc1)[C@@H](C)O]

Another sphingolipid metabolite mimetic is N-((2S,3R)-3-Hydroxy-1-oxo-1-(benzamino)butan-2-yl)-tetradecyl acid amide (herein referred to as NZJU19), the structure of which is shown below.

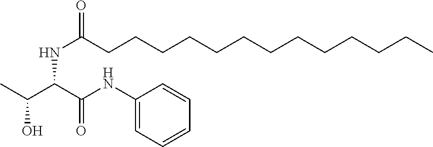 [SMILES: CCCCCCCCCCCCCC(=O)N[C@H](C(=O)Nc1ccccc1)[C@@H](C)O]

Another sphingolipid metabolite mimetic is (E)-N-((2S,3R)-3-hydroxy-1-oxo-1-(dodecylamino)butan-2-yl)-3-phenyl acrylic acid amide. (herein referred to as NZJU24 or NZJU2d), the structure of which is shown below.

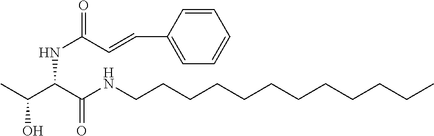 [SMILES: CCCCCCCCCCCCNC(=O)[C@@H](NC(=O)/C=C/c1ccccc1)[C@@H](C)O]

Another sphingolipid metabolite mimetic is (E)-N-((2S,3R)-3-hydroxy-1-oxo-1-(tetradecylamino)butan-2-yl)-3-phenyl acrylic acid amide (herein referred to as NZJU25 or NZJU3d), the structure of which is shown below.

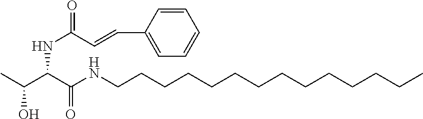 [SMILES: CCCCCCCCCCCCCCNC(=O)[C@@H](NC(=O)/C=C/c1ccccc1)[C@@H](C)O]

Another sphingolipid metabolite mimetic is (E)-N-((2S,3R)-3-hydroxy-1-oxo-1-(octadecylamino)butan-2-yl)-3-phenyl acrylic acid amide (herein referred to as NZJU26 or NZJU4d), the structure of which is shown below.

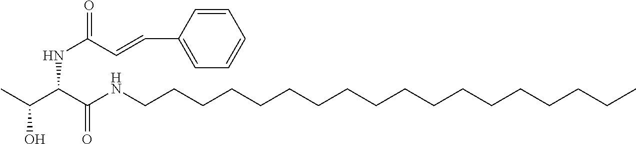 [SMILES: CCCCCCCCCCCCCCCCCCNC(=O)[C@@H](NC(=O)/C=C/c1ccccc1)[C@@H](C)O]

Another sphingolipid metabolite mimetic is N-((2S,3R)-3-hydroxy-1-oxo-1-(dodecylamino)butan-2-yl)-indole-3-acetic acid amide (herein referred to as NZJU27 or NZJU2e), the structure of which is shown below.

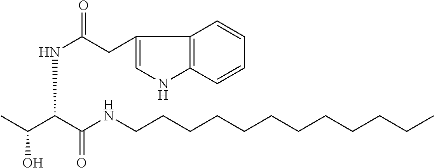 [SMILES: CCCCCCCCCCCCNC(=O)[C@@H](NC(=O)Cc1c[nH]c2ccccc12)[C@@H](C)O]

Another sphingolipid metabolite mimetic is N-((2S,3R)-3-hydroxy-1-oxo-1-(tetradecylamino)butan-2-yl)-indole-3-acetic acid amide (herein referred to as NZJU28 or NZJU3e), the structure of which is shown below.

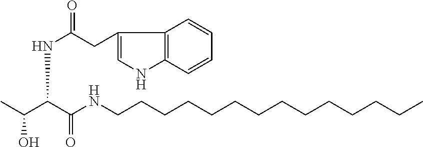 [SMILES: CCCCCCCCCCCCCCNC(=O)[C@@H](NC(=O)Cc1c[nH]c2ccccc12)[C@@H](C)O]

Another sphingolipid metabolite mimetic is N-((2S,3R)-3-hydroxy-1-oxo-1-(octadecylamino)butan-2-yl)-indole-3-acetic acid amide (herein referred to as NZJU29 or NZJU4e), the structure of which is shown below.

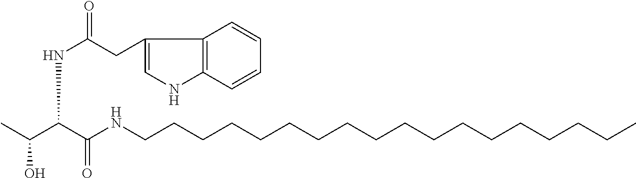 [SMILES: CCCCCCCCCCCCCCCCCCNC(=O)[C@@H](NC(=O)Cc1c[nH]c2ccccc12)[C@@H](C)O]

Another sphingolipid metabolite mimetic is N-((2S,3R)-3-hydroxy-1-oxo-1-(dodecylamino)butan-2-yl)-isoquinoline-1-carboxamide (herein referred to as NZJU30 or NZJU2f), the structure of which is shown below.

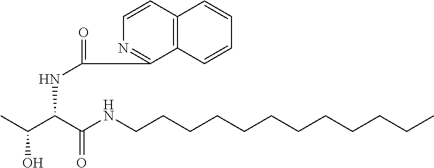 [SMILES: CCCCCCCCCCCCNC(=O)[C@@H](NC(=O)c1nccc2ccccc12)[C@@H](C)O]

Another sphingolipid metabolite mimetic is N-((2S,3R)-3-hydroxy-1-oxo-1-(tetradecylamino)butan-2-yl)-isoquinoline-1-carboxamide (herein referred to as NZJU31 or NZJU3f), the structure of which is shown below.

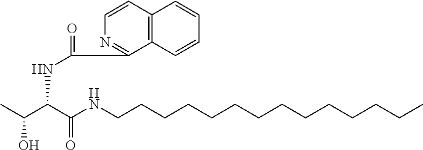 [SMILES: CCCCCCCCCCCCCCNC(=O)[C@@H](NC(=O)c1nccc2ccccc12)[C@@H](C)O]

Another sphingolipid metabolite mimetic is N-((2S,3R)-3-hydroxy-1-oxo-1-(octadecylamino)butan-2-yl)-isoquinoline-1-carboxamide (herein referred to as NZJU32 or NZJU4f), the structure of which is shown below.

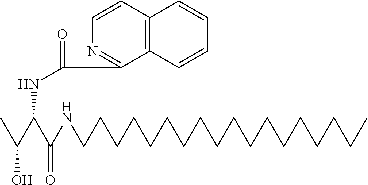 [SMILES: CCCCCCCCCCCCCCCCCCNC(=O)[C@@H](NC(=O)c1nccc2ccccc12)[C@@H](C)O]

Another sphingolipid metabolite mimetic is N-((2S,3R)-3-hydroxy-1-oxo-1-(dodecylamino)butan-2-yl)-2-thiophene carboxamide (herein referred to as NZJU33 or NZJU2g), the structure of which is shown below.

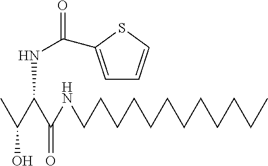 [SMILES: CCCCCCCCCCCCNC(=O)[C@@H](NC(=O)c1cccs1)[C@@H](C)O]

Another sphingolipid metabolite mimetic is N-((2S,3R)-3-hydroxy-1-oxo-1-(tetradecylamino)butan-2-yl)-2-thiophene carboxamide (herein referred to as NZJU34 or NZJU3g), the structure of which is shown below.

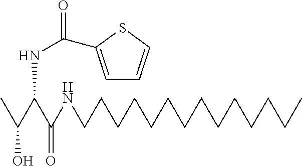 [SMILES: CCCCCCCCCCCCCCNC(=O)[C@@H](NC(=O)c1cccs1)[C@@H](C)O]

Another sphingolipid metabolite mimetic is N-((2S,3R)-3-hydroxy-1-oxo-1-(octadecylamino)butan-2-yl)-2-thiophene carboxamide (herein referred to as NZJU35 or NZJU4g), the structure of which is shown below.

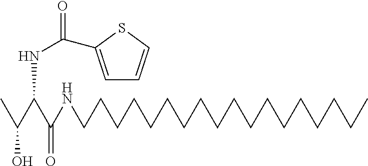 [SMILES: CCCCCCCCCCCCCCCCCCNC(=O)[C@@H](NC(=O)c1cccs1)[C@@H](C)O]

Another sphingolipid metabolite mimetic is N-((2S,3R)-3-hydroxy-1-oxo-1-(dodecylamino)butan-2-yl)-benzofuran-2-carboxamide (herein referred to as NZJU36 or NZJU2h), the structure of which is shown below.

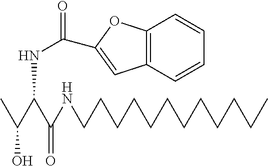 [SMILES: CCCCCCCCCCCCNC(=O)[C@@H](NC(=O)c1cc2ccccc2o1)[C@@H](C)O]

Another sphingolipid metabolite mimetic is N-((2S,3R)-3-hydroxy-1-oxo-1-(tetradecylamino)butan-2-yl)-benzofuran-2-carboxamide (herein referred to as NZJU37 or NZJU3h), the structure of which is shown below.

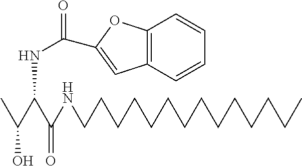 [SMILES: CCCCCCCCCCCCCCNC(=O)[C@@H](NC(=O)c1cc2ccccc2o1)[C@@H](C)O]

Another sphingolipid metabolite mimetic is N-((2S,3R)-3-hydroxy-1-oxo-1-(octadecylamino)butan-2-yl)-benzofuran-2-carboxamide (herein referred to as NZJU38 or NZJU4h), the structure of which is shown below.

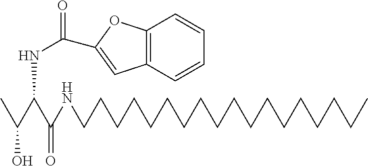 [SMILES: CCCCCCCCCCCCCCCCCCNC(=O)[C@@H](NC(=O)c1cc2ccccc2o1)[C@@H](C)O]

Another sphingolipid metabolite mimetic is N-((2S,3R)-3-hydroxy-1-oxo-1-(octylamino)butan-2-yl)-benzamide (herein referred to as NZJU39 or NZJU1a), the structure of which is shown below.

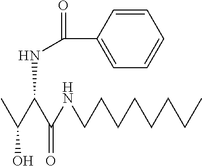 [SMILES: CCCCCCCCNC(=O)[C@@H](NC(=O)c1ccccc1)[C@@H](C)O]

Another sphingolipid metabolite mimetic is N-((2S,3R)-3-hydroxy-1-oxo-1-(octylamino)butan-2-yl)-Furan-2-carboxamide (herein referred to as NZJU40 or NZJU1b), the structure of which is shown below.

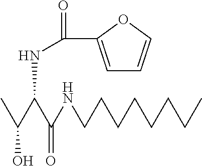 [SMILES: CCCCCCCCNC(=O)[C@@H](NC(=O)c1ccco1)[C@@H](C)O]

Another sphingolipid metabolite mimetic is (2E,4E)-N-((2S,3R)-3-hydroxy-1-oxo-1-(octylamino)butan-2-yl)-2,4-hexadienoic acid amide (herein referred to as NZJU41 or NZJU1c), the structure of which is shown below.

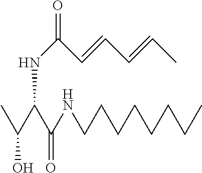 [SMILES: C/C=C/C=C/C(=O)N[C@H](C(=O)NCCCCCCCC)[C@@H](C)O]

Another sphingolipid metabolite mimetic is (E)-N-((2S,3R)-3-hydroxy-1-oxo-1-(octylamino)butan-2-yl)-3-phenyl acrylic acid amide (herein referred to as NZJU42 or NZJU1d), the structure of which is shown below.

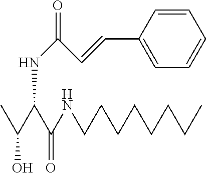 [SMILES: CCCCCCCCNC(=O)[C@@H](NC(=O)/C=C/c1ccccc1)[C@@H](C)O]

Another sphingolipid metabolite mimetic is N-((2S,3R)-3-hydroxy-1-oxo-1-(octylamino)butan-2-yl)-isoquinoline-1-carboxamide (herein referred to as NZJU43 or NZJU1f), the structure of which is shown below.

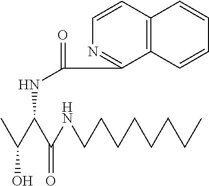 [SMILES: CCCCCCCCNC(=O)[C@@H](NC(=O)c1nccc2ccccc12)[C@@H](C)O]

Another sphingolipid metabolite mimetic is N-((2S,3R)-3-hydroxy-1-oxo-1-(octylamino)butan-2-yl)-2-thiophene carboxamide (herein referred to as NZJU44 or NZJU1g), the structure of which is shown below.

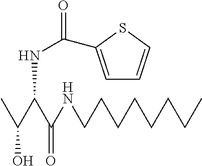 [SMILES: CCCCCCCCNC(=O)[C@@H](NC(=O)c1cccs1)[C@@H](C)O]

Another sphingolipid metabolite mimetic is N-((2S,3R)-3-hydroxy-1-oxo-1-(octylamino)butan-2-yl)-benzofuran-2-carboxamide (herein referred to as NZJU45 or NZJU1h), the structure of which is shown below.

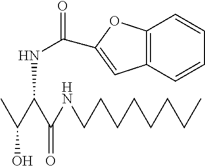 [SMILES: CCCCCCCCNC(=O)[C@@H](NC(=O)c1cc2ccccc2o1)[C@@H](C)O]

Another sphingolipid metabolite mimetic is (2S,3R)-2-Amino-3-hydroxy-N-tetradecylbutanamide (herein referred to as NZJU46 or NZJU3), the structure of which is shown below.

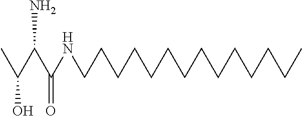 [SMILES: CCCCCCCCCCCCCCNC(=O)[C@@H](N)[C@@H](C)O]

Another sphingolipid metabolite mimetic is N-((2S,3R)-3-hydroxy-1-oxo-1-(dodecylamino)butan-2-yl)-indole-2-carboxamide (herein referred to as NZJU47 or NZJU2i), the structure of which is shown below.

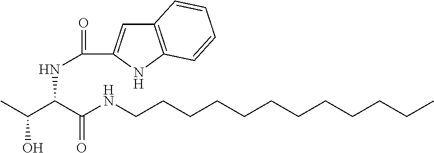 [SMILES: CCCCCCCCCCCCNC(=O)[C@@H](NC(=O)c1cc2ccccc2[nH]1)[C@@H](C)O]

Another sphingolipid metabolite mimetic is N-((2S,3R)-3-hydroxy-1-oxo-1-(tetradecylamino)butan-2-yl)-indole-2-carboxamide (herein referred to as NZJU48 or NZJU3i), the structure of which is shown below.

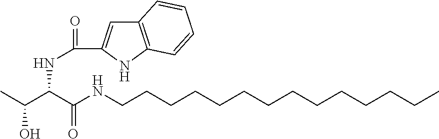 [SMILES: CCCCCCCCCCCCCCNC(=O)[C@@H](NC(=O)c1cc2ccccc2[nH]1)[C@@H](C)O]

Another sphingolipid metabolite mimetic is N-((2S,3R)-3-hydroxy-1-oxo-1-(octadecylamino)butan-2-yl)-indole-2-carboxamide (herein referred to as NZJU49 or NZJU4i), the structure of which is shown below.

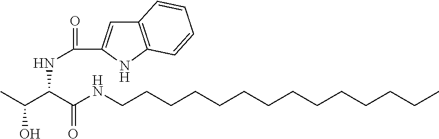 [SMILES: CCCCCCCCCCCCCCNC(=O)[C@@H](NC(=O)c1cc2ccccc2[nH]1)[C@@H](C)O]

Another sphingolipid metabolite mimetic is N-((2S,3R)-3-hydroxy-1-oxo-1-(dodecylamino)butan-2-yl)-(N-methyl)indole-2-carboxamide (herein referred to as NZJU50 or NZJU2j), the structure of which is shown below.

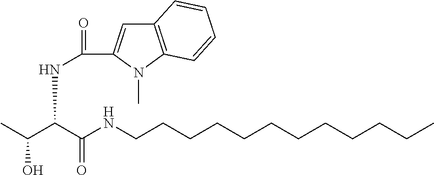 [SMILES: CCCCCCCCCCCCNC(=O)[C@@H](NC(=O)c1cc2ccccc2n1C)[C@@H](C)O]

Another sphingolipid metabolite mimetic is N-((2S,3R)-3-hydroxy-1-oxo-1-(tetradecylamino)butan-2-yl)-(N-methyl)indole-2-carboxamide (herein referred to as NZJU51 or NZJU3j), the structure of which is shown below.

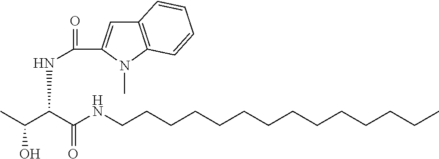 [SMILES: CCCCCCCCCCCCCCNC(=O)[C@@H](NC(=O)c1cc2ccccc2n1C)[C@@H](C)O]

Another sphingolipid metabolite mimetic is N-((2S,3R)-3-hydroxy-1-oxo-1-(octadecylamino)butan-2-yl)-(N-methyl)indole-2-carboxamide (herein referred to as NZJU52 or NZJU4j), the structure of which is shown below.

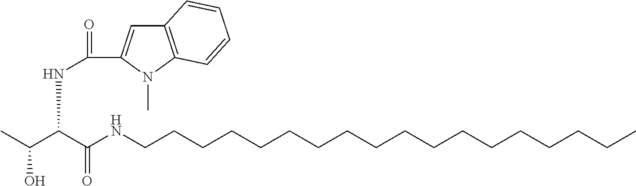 [SMILES: CCCCCCCCCCCCCCCCCCNC(=O)[C@@H](NC(=O)c1cc2ccccc2n1C)[C@@H](C)O]

Another sphingolipid metabolite mimetic is N-((2S,3R)-3-hydroxy-1-oxo-1-(dodecylamino)butan-2-yl)-6-hydroxylnaphthoic-2-carboxamide (herein referred to as NZJU53), the structure of which is shown below.

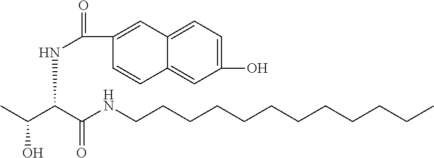 [SMILES: CCCCCCCCCCCCNC(=O)[C@@H](NC(=O)c1ccc2cc(O)ccc2c1)[C@@H](C)O]

Another sphingolipid metabolite mimetic is N-((2S,3R)-3-hydroxy-1-oxo-1-(tetradecylamino)butan-2-yl)-6-hydroxylnaphthoic-2-carboxamide (herein referred to as NZJU54), the structure of which is shown below.

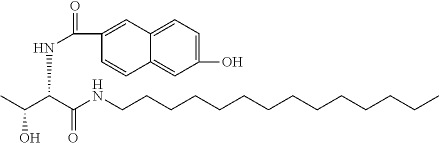 [SMILES: CCCCCCCCCCCCCCNC(=O)[C@@H](NC(=O)c1ccc2cc(O)ccc2c1)[C@@H](C)O]

Another sphingolipid metabolite mimetic is N-((2S,3R)-3-hydroxy-1-oxo-1-(octadecylamino)butan-2-yl)-6-hydroxylnaphthoic-2-carboxamide (herein referred to as NZJU55), the structure of which is shown below.

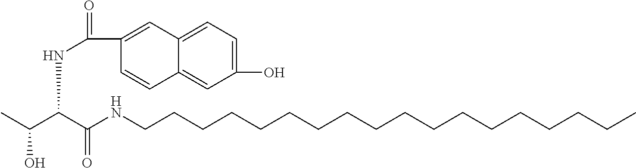 [SMILES: CCCCCCCCCCCCCCCCCCNC(=O)[C@@H](NC(=O)c1ccc2cc(O)ccc2c1)[C@@H](C)O]

Another sphingolipid metabolite mimetic is N-((2S,3R)-3-hydroxy-1-oxo-1-(dodecylamino)butan-2-yl)-4-methylbenzsulfonamide (herein referred to as NZJU56), the structure of which is shown below.

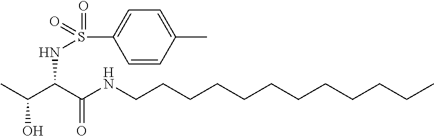 [SMILES: CCCCCCCCCCCCNC(=O)[C@@H](NS(=O)(=O)c1ccc(C)cc1)[C@@H](C)O]

Another sphingolipid metabolite mimetic is N-((2S,3R)-3-hydroxy-1-oxo-1-(tetradecylamino)butan-2-yl)-4-methylbenzsulfonamide (herein referred to as NZJU57), the structure of which is shown below.

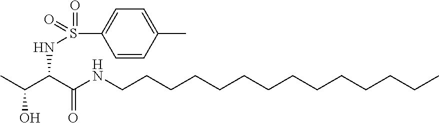 [SMILES: CCCCCCCCCCCCCCNC(=O)[C@@H](NS(=O)(=O)c1ccc(C)cc1)[C@@H](C)O]

Another sphingolipid metabolite mimetic is N-((2S,3R)-3-hydroxy-1-oxo-1-(octadecylamino)butan-2-yl)-4-methylbenzsulfonamide (herein referred to as NZJU58), the structure of which is shown below.

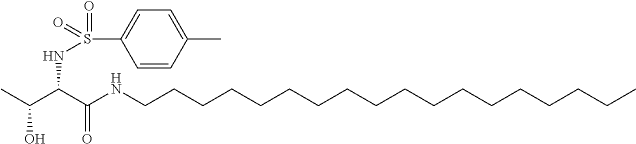 [SMILES: CCCCCCCCCCCCCCCCCCNC(=O)[C@@H](NS(=O)(=O)c1ccc(C)cc1)[C@@H](C)O]

Another sphingolipid metabolite mimetic is N-((2S,3R)-3-hydroxy-1-oxo-1-(dodecylamino)butan-2-yl)-2-thiophene sulfonamide (herein referred to as NZJU59), the structure of which is shown below.

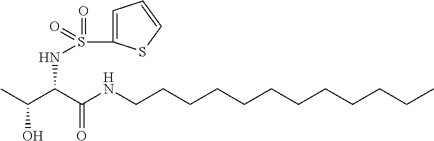 [SMILES: CCCCCCCCCCCCNC(=O)[C@@H](NS(=O)(=O)c1cccs1)[C@@H](C)O]

Another sphingolipid metabolite mimetic is N-((2S,3R)-3-hydroxy-1-oxo-1-(tetradecylamino)butan-2-yl)-2-thiophene sulfonamide (herein referred to as NZJU60), the structure of which is shown below.

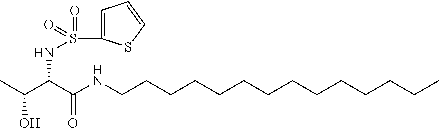 [SMILES: CCCCCCCCCCCCCCNC(=O)[C@@H](NS(=O)(=O)c1cccs1)[C@@H](C)O]

Another sphingolipid metabolite mimetic is N-((2S,3R)-3-hydroxy-1-oxo-1-(octadecylamino)butan-2-yl)-2-thiophene sulfonamide (herein referred to as NZJU61), the structure of which is shown below.

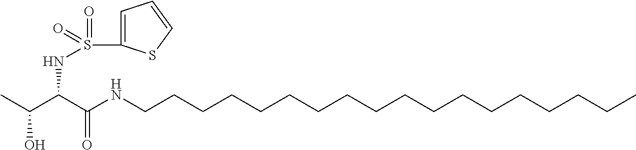 [SMILES: CCCCCCCCCCCCCCCCCCNC(=O)[C@@H](NS(=O)(=O)c1cccs1)[C@@H](C)O]

Another sphingolipid metabolite mimetic is N-((2S,3R)-3-hydroxy-1-oxo-1-(tetradecylamino)butan-2-yl)-2-naphthylsulfonamide (herein referred to as NZJU62), the structure of which is shown below.

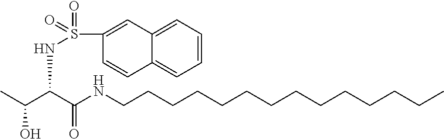 [SMILES: CCCCCCCCCCCCCCNC(=O)[C@@H](NS(=O)(=O)c1ccc2ccccc2c1)[C@@H](C)O]

Another sphingolipid metabolite mimetic is N-((2S,3R)-3-hydroxy-1-oxo-1-(octadecylamino)butan-2-yl)-2-naphthylsulfonamide (herein referred to as NZJU63), the structure of which is shown below.

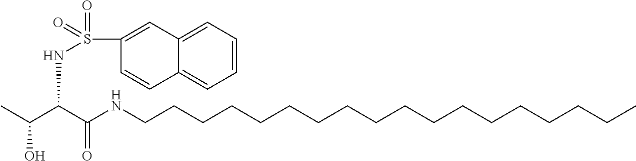 [SMILES: CCCCCCCCCCCCCCCCCCNC(=O)[C@@H](NS(=O)(=O)c1ccc2ccccc2c1)[C@@H](C)O]

Another sphingolipid metabolite mimetic is (3S,4R,5S)-3,4,5-trihydroxy-N-((2S,3R)-3-hydroxy-1-oxo-1-(tetradecylamino)butan-2-yl)cyclohex-1-enecarboxamide (herein referred to as NZJU64), the structure of which is shown below.

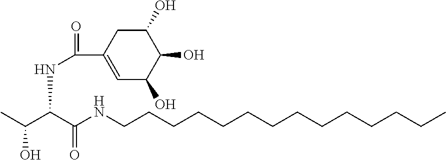 [SMILES: CCCCCCCCCCCCCCNC(=O)[C@@H](NC(=O)C1=C[C@H](O)[C@H](O)[C@@H](O)C1)[C@@H](C)O]

Although certain optical isomers have been described above, the disclosed sphingolipid metabolite mimetics include all possible stereoisomers. One of skill in the art can readily discern from the structures of the sphingolipid mimetics provided herein the various stereoisomers for each compound. For example, the disclosure of (2S,3R)-2-Amino-3-hydroxy-N-dodecylbutanamide should be construed as the separate disclosure of (2S,3S)-2-Amino-3-hydroxy-N-dodecylbutanamide, (2R,3R)-2-Amino-3-hydroxy-N-dodecylbutanamide, and (2R,3S)-2-Amino-3-hydroxy-N-dodecylbutanamide as well. Likewise, the disclosure of any one of the compounds having a structure shown below should be construed as the separate disclosure of each and every one of the compounds identified by structure below.

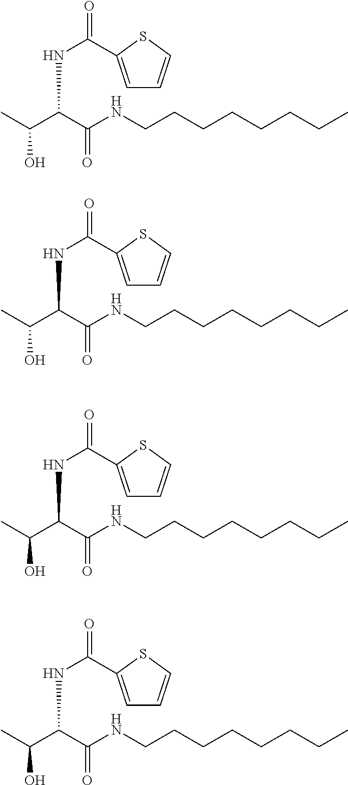

In addition, the disclosure of any generic formula should be construed as the separate disclosure of each and every possible stereoisomer of the disclosed generic formula. Any specific compound, including any specific stereoisomer, may be explicitly disclaimed or excluded. Likewise any specific enantiomer or stereoisomer of any generic formula disclosed may be explicitly disclaimed or excluded. In addition, any specific substituent disclosed in any generic formula may be explicitly excluded.

The disclosed sphingolipid metabolite mimetics include the compounds described herein as NZJU13(NZJU2b), NZJU16(NZJU2a), NZJU26 (NZJU4d), NZJU27(NZJU2e), NZJU28(NZJU3e), NZJU29(NZJU4e), NZJU30(NZJU2f), NZJU33(NZJU2g), NZJU34(NZJU3g), NZJU36(NZJU2h), NZJU37(NZJU3h), NZJU39(NZJU1a), NZJU41(NZJU1c), NZJU44(NZJU1g), NZJU45(NZJU1h), NZJU46, NZJU47(NZJU2i), NZJU48(NZJU3i), NZJU51(NZJU3j), NZJU57, NZJU60, and NZJU62. Preferred sphingolipid metabolite mimetics include, but are not limited to NZJU13(NZJU2b), NZJU16(NZJU2a), NZJU30(NZJU2f), NZJU34(NZJU3g), NZJU41(NZJU1c), NZJU44(NZJU1g), and NZJU45(NZJU1h).

To date, the synthetic sphingolipid metabolite mimetics have primarily focused on analogues of the ceramides and sphingosines, as these are expected to be involved in the proliferative and apoptotic signaling pathways. Examples of synthetic ceramide mimetics include those described in Padrón, J. M, Current Medicinal Chemistry 2006, 13:755-770; Shikata, K. et al., Tetrahedron 2002, 58:5803-5809; Shikata, K. et al, Bioorganic and Medicinal Chemistry Letters 2003, 13:613-616; Shikata, K. et al., Bioorganic and Medicinal Chemistry 2003, 11:2723-2728; Niro, H. et al., Bioorganic and Medicinal Chemistry 2004, 12:45-51; Rajan, R. et al., Chemistry and Biodiversity 2004, 1:1785-1799; Chun, J. et al., Journal of Organic Chemistry 2003, 68:355-359; Chun, J. et al., Journal of Organic Chemistry 2003, 68:348-354; Struckhoff, A. P. Journal of Pharmacology and Experimental Therapeutics 2004, 309:452-461; Chang, Y. T. Journal of the American Chemical Society 2002, 124:1856-1857 all of which are incorporated herein by reference. Examples of synthetic sphingosine mimetics include those described in Padrón, J. M, Current Medicinal Chemistry 2006, 13:755-770; Yatomi, et al., Biochemistry 1996, 35:626-633; Edsall, L. C. et al., Biochemistry 1998, 37:12892-12898; Sweeney, E. A. et al., International Journal of Cancer 1996, 66:358-366; Shirahama, T. et al. Clinical Cancer Research 1997, 3:257-264; Rajewski, R. A. et al. Journal of Pharmaceutical and Biomedical Analysis 1995, 13:247-253; Sachs, C. W. et al. Journal of Biological Chemistry 1995, 270:26639-26648; Schwartz, G. K. et al. Journal of the National Cancer Institute 1995, 87:1394-1399; Dragusin, M et al. Journal of Lipid Research 2003, 44:1772-1779; Mauer, B. J. Journal of the National Cancer Institute 2000, 92:1897-1909; Stewart, M. E. and Downing, D. T., Journal of Lipid Research 1999, 40:1434-1439; Bouwstra, J. A. et al. Journal of Lipid Research 2001, 42:1759-1770; Wakita, H. et al. Journal of Investigative Dermatology 1992, 99:617-622; Yadav, J. S. et al. Tetrahedron Letters 2003, 44:2983-2985; Padrón, J. M. et al. Bioorganic and Medicinal Chemistry Letters 1999, 9:821-826; Markidis, T. et al. Anticancer Research 2001, 21:2835-2840; Padrón, J. M. and Peters, G. J. Investigational New Drug—Journal of Anti-Cancer Agents 2006, 24:195-202; Padrón, J. M. et al. Critical Reviews in Oncology/Hematology 2000, 36:141-157; and Del Olmo, E. et al. Bioorganic and Medicinal Chemistry Letters 2002, 12:2621-2626 all of which are incorporated herein by reference.

B. Synthesis

Synthetic sphingolipid metabolite mimetics may be made using conventional techniques known in art. For example, the synthetic sphingolipid metabolite mimetics can be synthesized from a synthetic intermediate having the general formula:

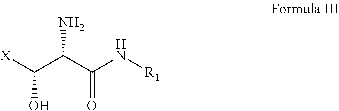

wherein R₁ and X taken independently may be a hydrogen atom, a halogen atom, a hydroxyl group, or any other organic groupings containing any number of carbon atoms and optionally including one or more heteroatoms such as oxygen, sulfur, or nitrogen grouping in linear, branched, or cyclic structural formats, representative R₁ and X groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group.

Alternatively, the sphingolipid metabolite mimetic can be synthesized from an intermediate represented by Formula III wherein X is selected from the group consisting of hydrogen, methyl, or alkyl; and R₁ is alkyl or substituted alkyl, preferably linear alkyl, preferably C₁₀-C₂₀ alkyl.

The sphingolipid metabolite mimetic can also be synthesized from an intermediate represented by Formula III wherein X is methyl or hydrogen, preferably methyl; and R₁ is linear alkyl, preferably a linear C₁₀-C₂₀ alkyl.

In certain embodiments one or more of the intermediates described above also function as a sphingolipid metabolite mimetic. Representative sphingolipid metabolite mimetics are represented by Formula III wherein R₁ and X taken independently may be a hydrogen atom, a halogen atom, a hydroxyl group, or any other organic groupings containing any number of carbon atoms and optionally including one or more heteroatoms such as oxygen, sulfur, or nitrogen grouping in linear, branched, or cyclic structural formats, representative R₁ and X groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group.

Still another sphingolipid metabolite mimetic is represented by Formula III wherein X is selected from the group consisting of hydrogen, methyl, or alkyl; and R₁ is alkyl or substituted alkyl, preferably linear alkyl, preferably C₁₀-C₂₀ alkyl.

Another sphingolipid metabolite mimetic is represented by Formula III wherein X is methyl or hydrogen, preferably methyl; and R₁ is linear alkyl, preferably a linear C₁₀-C₂₀ alkyl.

In certain embodiments the sphingolipid metabolite mimetic is synthesized from (2S,3R)-2-Amino-3-hydroxy-N-octylbutanamide (herein referred to as NZJU1), the structure of which is shown below.

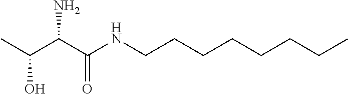 [SMILES: CCCCCCCCNC(=O)[C@@H](N)[C@@H](C)O]

(2S,3R)-2-Amino-3-hydroxy-N-octylbutanamide may be a sphingolipid metabolite mimetic in some embodiments.

Alternatively, the sphingolipid metabolite mimetic is synthesized from (2S,3R)-2-Amino-3-hydroxy-N-dodecylbutanamide (herein referred to as NZJU2), the structure of which is shown below.

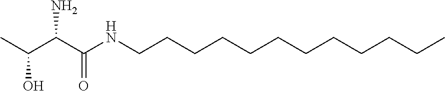 [SMILES: CCCCCCCCCCCCNC(=O)[C@@H](N)[C@@H](C)O]

In certain embodiments (2S,3R)-2-Amino-3-hydroxy-N-dodecylbutanamide is a sphingolipid metabolite mimetic.

The sphingolipid metabolite mimetic can also be synthesized from (2S,3R)-2-Amino-3-hydroxy-N-tetradecylbutanamide (herein referred to as NZJU46 or NZJU3), the structure of which is shown below.

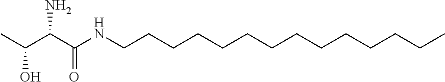 [SMILES: CCCCCCCCCCCCCCNC(=O)[C@@H](N)[C@@H](C)O]

(2S,3R)-2-Amino-3-hydroxy-N-tetradecylbutanamide may in some cases be a sphingolipid metabolite mimetic.

The sphingolipid metabolite mimetic can also be synthesized from (2S,3R)-2-Amino-3-hydroxy-N-octadecylbutanamide (herein referred to as NZJU4), the structure of which is shown below.

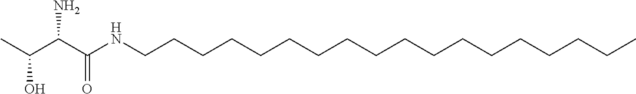 [SMILES: CCCCCCCCCCCCCCCCCCNC(=O)[C@@H](N)[C@@H](C)O]

In some instances (2S,3R)-2-Amino-3-hydroxy-N-octadecylbutanamide is a sphingolipid metabolite mimetic.

Sphingolipid metabolite mimetics may be synthesized using the intermediates described above employing a number of synthetic strategies known to those of skill in the art to obtain the desired compound. Sphingolipid metabolite mimetics can be synthesized through the combining, adding into, or mixing of any of the intermediates described above with one or more, preferably one, organic acid. An exemplary organic acid is an alkenyl-functionalized carboxylic acid, preferably a linear alkenyl-functionalized carboxylic acid, more preferably a linear C₃-C₇ alkenyl substituted carboxylic acid. Specific organic acids include, but are not limited to acetic acid, acrylic acid, (E)-but-2-enoic acid, (E)-penta-2,4-dienoic acid, (2E,4E)-hexa-2,4-dienoic acid, (2E,4E)-hepta-2,4,6-trienoic acid, (2E,4E,6E)-octa-2,4,6-trienoic acid, or suitable derivative s thereof. The organic acid can be an aryl, heteroaryl, or fused heteroaryl carboxylic acid including but not limited to benzoic acid, furan-2-carboxylic acid, cinnamic acid, 2-(1H-indol-3-yl)acetic, isoquinoline-1-carboxylic acid, thiophene-2-carboxylic acid, benzofuran-2-carboxylic acid, 1H-indole-2-carboxylic acid, 1-methyl-1H-indole-2-carboxylic acid, and suitable substitutions or derivatives thereof.

The sphingolipid metabolite mimetic can be synthesized from one or more of the intermediates described above according to the following scheme:

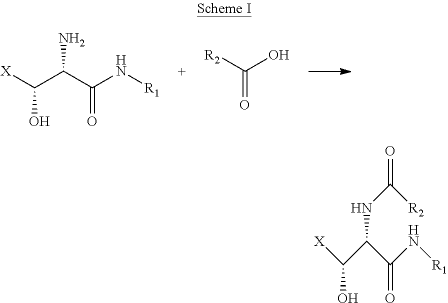

In certain embodiments the reaction according to Scheme I is performed at or below room temperature. In an exemplary reaction according to Scheme I, the reaction is performed at 0° C. for 2 hours followed by more than 8 hours at approximately room temperature. The reaction can also be performed in the presence of one or more dehydration agents that are useful for activating carboxylic acids. An exemplary dehydration agent is a carbodiimide, including but not limited to 1-Ethyl-3-[3-dimethylaminopropyl]carbodiimide hydrochloride (EDC.HCl), N,N′-dicyclohexylcarbodiimide (DCC), and N,N′-Diisopropylcarbodiimide (DIC). Alternatively, the reaction according to Scheme I is performed under slightly basic conditions for example at pH 8-9.

The synthetic sphingolipid metabolite mimetics produced according to the methods and reactions described above may be recovered, obtained, isolated, extracted, purified, crystallized, or separated by conventional methods known to those of skill in the art.

In one embodiment the intermediates useful for the synthesis of one or more sphingolipid metabolite mimetics are synthesized starting from a suitable α-amino acid or substituted α-amino acid wherein the amine is functionalized with one or more protecting groups.

In certain embodiments intermediates useful for synthesizing one or more sphingolipid metabolite mimetics are synthesized from starting materials having the general formula:

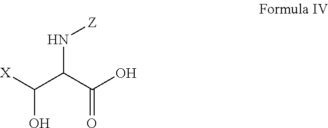

wherein X may be a hydrogen atom, a halogen atom, a hydroxyl group, or any other organic groupings containing any number of carbon atoms and optionally including one or more heteroatoms such as oxygen, sulfur, or nitrogen grouping in linear, branched, or cyclic structural formats, representative X groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; and Z may be any amine-protecting group suitable for protecting the amine under reaction conditions.

The intermediates useful for the synthesis of one or more sphingolipid metabolite mimetics can be synthesized from at least one starting materials according to Formula IV wherein X is hydrogen or methyl and Z is a amine-protecting group selected from group consisting of t-butyl carbamate, 9-fluorenmethyl carbamate, benzyl carbamate, acetamide, trifluoroacetamide, phthalimide, benzylamine, triphenylmethylamine, benzylideneamine, p-toluenesulfonamide, or suitable derivatives thereof.

The intermediates useful for the synthesis of one or more sphingolipid metabolite mimetics can be synthesized through the combining, adding into, or mixing of one or more materials according to Formula IV with one or more primary amines; wherein X is hydrogen or methyl, Z is an amine-protecting group selected from group consisting of t-butyl carbamate, 9-fluorenylmethyl carbamate, benzyl carbamate, acetamide, trifluoroacetamide, phthalimide, benzylamine, triphenylmethylamine, benzylideneamine, p-toluenesulfonamide, or suitable derivatives thereof. In particularly preferred embodiments the primary amine is an alkyl-substituted amine, preferably a linear alkyl-substituted amine, most preferably a C₁₀-C₂₀ linear alkyl-substituted amine. In certain embodiments the intermediates are synthesized at or below room temperature, for example at 0° C. for 2 hours followed by more than 8 hours at approximately room temperature. The intermediates can also be synthesized in the presence of one or more dehydration agents that are useful for activating carboxylic acids including but not limited to a carbodiimide. Exemplary carbodiimide include but are not limited to 1-Ethyl-3-[3-dimethylaminopropyl]carbodiimide hydrochloride (EDC.HCl), N,N′-dicyclohexylcarbodiimide (DCC), and N,N′-Diisopropylcarbodiimide (DIC). The intermediates are synthesized under slightly basic conditions. In particular embodiments the intermediates are synthesized wherein the pH is adjusted to approximately 8-9.

The intermediates produced according to the methods and reactions described above may be recovered, obtained, isolated, extracted, purified, crystallized, or separated by any suitable methods known in the art.

Synthetic sphingolipid metabolite mimetics can be made using conventional techniques known in the art. Examples of synthetic strategies include those described in Bielawska, A. et al. Journal of Biological Chemistry 1996, 271:12646-12654; Grijalvo, S. et al. Chemistry and Physics of Lipids 2006, 144:69-84; Sugita, M et al., Biochimica et Biophysica Acta 1975, 398:125-131; Xarnod, C. et al. Tetrahedron 2012, 68:6688-6695; Mu, Y. et al. European Journal of Organic Chemistry 2012, 13:2614-2620; Singh, A. et al. Bioorganic and Medicinal Chemistry 2011, 19:6174-6181; Lee, S., et al. Organic and Biomolecular Chemistry 2011, 9:4580-4586; Morales-Sema, J. A. et al. Current Organic Chemistry 2010, 14:2483-2521; Koroniak, K. et al. Synthesis-Stuttgart 2010, 19:3309-3314; Grijalvo, S. et al. European Jouranl of Organic Chemistry 2008, 1:150-155; Ha, H. J. et al. Bioorganic and Medicinal Chemistry Letters 2006, 16:1880-1883; Liao, J. Y. et al. Tetrahedron 2005, 61:4715-4733; Brockman, H. L. et al. Biophysical Journal 2004, 87:1722-1731; and Menaldino, D. S. et al. Pharmacological Research 2003, 47:373-381 all of which are incorporated herein by reference.

C. Sphingolipid Metabolites and Cancer

The disclosed sphingolipid metabolite mimetics are useful for elucidating the metabolic pathways involved in sphingolipid signaling, sphingolipid metabolism and in the treatment of proliferative diseases and disorders including cancer. Sphingolipids are the most structurally diverse as well as complex category of lipids due to their numerous variations in the sphingoid bases, N-acyl linked fatty acids and head groups (Vesper, H., et al., The Journal of Nutrition 129: 1239-1250). Sphingolipids are bioeffectors known to mediate various cellular processes, including proliferation and apoptosis of cancer cells (Baran et al. Journal of Biological Chemistry, 2007, 282:10922-10934; Elojeimy et al. Molecular Therapeutics, 2007; 15:1259-1263; Kolesnick and Fuks Oncogene, 2003, 22:5897-5906; Ogretmen B. FEBS Letters, 2006, 580:5467-5476; Oskouian et al. Proceedings of the National Academy of Science USA, 2006; 103: 17384-17389; Ponnusamy et al. Future Oncology, 2010, 6:1603-1624; Aoyagi, T. et al. Lymphatic Research and Biology 2012, 10:97-106; Apraiz, A. et al. BMC Cancer 2011, 11:477; Barcelo-Coblijn et al. Proceedings of the National Academy of Science USA 2011, 108(49):19569-19574; Berra et al. European Journal of Cancer Prevention, 2002, 11(2):193-197; Bini et al. Neuropharmacology, 2012, 63(4) 524-537; Chatzakos et al. Biochemical Pharmacology, 2012, 84(5):712-721; Duan and Nilsson Progress in Lipid Researhc, 2009, 48(1):62-72; Furuya et al. Cancer Metastasis Reviews, 2011, 30:567-576; Gault et al. Journal of Biological Chemistry, 2012, 287:31794-31803; Kamocki et al. American Journal of Respiratory Cell and Molecular Biology, 2012; Kent et al. Lipids, 2008, 43:143-149; Korbelik et al. Photochemical and Photobiological Sciences, 2012; Kumar et al. Carcinogenesis, 2012, 33:291-295; Morad et al. Molecular Cancer Therapeutics, 2012; Morales and Fernandez-Checa Mini-Reviews in Medicinal Chemistry, 2007, 7:371-382; Patwardhan and Liu Progress in Lipid Research, 2011, 50:104-114; Radin N. S. Urology, 2002, 60:562-568; Radin N. S. Cancer Investigation, 2002, 20:779-786; Russo et al. Journal of Clinical Investigations, 2012; Saddoughi et al. Subcellular Biochemistry, 2008, 49:413-440; Santos and Schulze FEBS Journal, 2012, 279:2610-2623; Schmelz Frontiers in Bioscience, 2004, 9:2632-2639; Segui et al. Biochemica et Biophysica Acta, 2006, 1758:2104-2120; Senkal et al. Faseb Journal, 2010, 24:296-308; Simon et al. Food & Function, 2010, 1:90-98; Simon et al. Molecular Nutrition and Food Research, 2009, 53:332-340; Spassieva and Bieberich Anticancer Agents in Medicinal Chemistry, 2011, 11:882-890; Tilly and Kolesnick Biochemical et Biophysica Acta, 2002, 1585:135-138)

While sphingolipids are present in most foods, biosynthetic sphingolipids are essential for human survival as the sphingoid bases are typically degraded in the mammalian digestive tract. The ceramides are central to the biosynthesis of the vast majority of sphingolipid metabolites, and interconversion between many of the metabolic forms is relatively facile. Interestingly, the different metabolites often exhibit differing or even competing activities. Many of the most important sphingolipid metabolites, including for instance ceramide, sphingosine, and sphingosine-1-phosphate, are interconvertible. This has complicated the process of determining their specific roles played as bioeffectors in various cellular processes. Sphingolipids have traditionally been considered simply structural components of cellular membranes, but in recent years have become appreciated as bioeffectors controlling signaling in processes such as proliferation, growth migration, differentiation, and apoptosis. Additionally, sphingolipids have been implicated in several diseases such as cancer, obesity, atherosclerosis and sphingolipidoses (Ozbayraktar, F. et al., 2009. Biotechnology journal 4: 1028-1041; Vance, D. et al., 1967. Journal of lipid research 8: 621-630; Bartke, N., et al., 2009. Journal of lipid research 50 Suppl: S91-96; Cowart, L. A. 2009. Trends in endocrinology and metabolism: TEM 20: 34-42; Lahiri, S. et al., 2007. Cell Mol Life Sci 64: 2270-2284; Liliom, K. et al., 2001. The Biochemical journal 355: 189-197; Merrill, A. H. et al., 2009. Journal of lipid research 50 Suppl: S97-102; Wymann, M. P. et al., 2008. Nature reviews 9: 162-176). Thus, it has become increasingly necessary to establish the metabolomic profile of sphingolipids to understand how sphingolipid biosynthesis and turnover regulate cell function under normal and abnormal conditions, how perturbations in one sphingolipid metabolite may enhance or interfere with the action of another, and where and how all these sphingolipid metabolites are made and removed.

Mammalian sphingolipid metabolites typically vary in the presence or absence of: (i) the 4,5-trans-double bond (for example, sphingosine has a double bond whereas sphinganine (also referred to as dihydrosphingosine) does not); (ii) double bond (s) at other positions, such as position 8; (iii) a hydroxyl group at position 4 (D-1-hydroxysphinganine, also called“phytosphingosine”) or elsewhere (Robson et al., J. Lipid Res. 35: 2060-2068, 1994); (iv) methyl group (s) on the alkyl side chain or on the amino group, such as N,N-dimethylsphingosine; and (v) acylation of the amino group (for example ceramide (also referred to as N-acylsphingosine), and dihydroceramide (also referred to as N-acyl-sphinganine)). The 4-hydroxysphinganines are the major long-chain bases of yeast (Wells, G. B. and Lester, R. L., J. Biol. Chem., 258: 10200-10203, 1983), plants (Lynch, D. V., Lipid Metabolism in Plants (T. S. Moore, Jr., ed.), pp. 285-308, CRC Press, Boca Raton, Fla. 1993), and fungi (Merrill et al., Fungal Lipids (R. Prasad and M. Ghanoum, eds.), CRC Press, Boca Raton, Fla., 1995a), but are also made by mammals (Crossman and Hirschberg, J. Biol. Chem, 252: 5815-5819, 1977). Other modifications of the long-chain base backbone include phosphorylation at the hydroxyloxygen of carbon 1 (Buehrer and Bell, Adv. Lipid Res. 6: 59-67, 1993), and acylation (Merrill and Wang, Methods Enzymol,. 209: 427-437, 1992) (Igarashi and Hakomori, Biochim. Biophys. Res. Commun. 164: 1411-1416, 1989; Felding-Habermann et al., Biochemistry 29: 6314-6322, 1990) of the amino group. Each of these compounds can be found in various alkyl chain lengths, with 18 carbons predominating in most sphingolipids, but other homologs can constitute a major portion of specific sphingolipid (as exemplified by the large amounts of C₂₀ sphingosine in brain gangliosides) (Valsecchi et al., J. Neurochem., 60: 193-196, 1993) and in different sources (e.g., C₁₆ sphingosine is a substantial component of milk sphingomyelin) (Morrison, Biochim. Biophys. Acta., 176: 537-546, 1969).

Several sphingolipid metabolites, especially ceramide (Cer), sphingosine (Sph), sphingosine 1-phosphate (S1P) and ceramide 1-phosphate (Cer1P), have been identified as bioactive signaling molecules that control cell growth and death (Cuvillier, O., et al., Nature 381: 800-803; Hannun, Y. A. 1996, Science 274: 1855-1859); Mathias, S., et al., 1998. The Biochemical journal 335 (Pt 3): 465-480; Spiegel, S et al., 1996. Faseb J 10: 1388-1397; Vesper, H., et al., 1999. The Journal of nutrition 129: 1239-1250; Ichi, I., et al., 2007. Nutrition 23: 570-574; Katsikas, H., et al., 1995. Biochimica et biophysica acta 1258: 95-100; Ozbayraktar, F. et al., 2009. Biotechnology journal 4: 1028-1041; Vance, D. et al., 1967. Journal of lipid research 8: 621-630; Hammad, S. M., et al. 2008. Prostaglandins & other lipid mediators 85: 107-114(1-10)). Ceramides have been found to mediate Fas receptor clustering, capping and activation to promote Fas-mediated apoptosis (Cremesti et al. Journal of Biological Chemistry, 2001, 276:23954-23961; Grassme et al. Oncogene, 2003, 22:5457-5470; Park et al. Cancer Biology & Therapy, 2008, 7:1648-1662; Park et al. Molecular Cancer Therapeutics, 2008, 7:2633-2648; Castro et al. Biophysics Journal, 2011, 101:1632-1641; Corre et al. Mutation Research, 2010, 704:61-67; Martin et al. Cancer Research, 2005, 65:11447-11458). Whereas sphingosin-1-phosphatase generally decreases cancer cell sensitivity to apoptosis and thus promotes tumor growth and progression. The presence of elevated levels of glucosylceramide and other glycosphingolipids is closely linked to cancer cell drug resistance and metastasis. Sphingolipid metabolites can thus be understood as important targets in understanding the signaling pathways leading to drug resistant and metastatic cancers. The synthetic sphingolipid metabolite mimetics and methods of synthesizing described herein will not only provide a powerful doorway to elucidate these pathways, but may also provide powerful therapeutics for treating cancer, and in particular for treating difficult metastatic and drug-resistant cancers.

### II. Compositions and Formulations

Compositions for treating cancer or tumor cells include one or more active agents. Active agents include sphingolipid metabolite mimetics, death receptor agonists, and other therapeutic agents, for example anti-inflammatories, anti-infective agents, chemotherapeutic agents or anti-neoplastic agents. In some embodiments, compositions contain multiple active agents. In other embodiments, active agents are administered in separate compositions, either simultaneously or at different times.

A. Sphingolipid Metabolite Mimetics The compositions can contain one or more sphingolipid metabolite mimetics represented by Formula I, wherein R₁, R₂, and X may be taken independently as described above. In some instances the compositions contain an enantiomer, derivative, prodrug, or salt of one or more of the sphingolipid metabolite mimetics described herein.

The compositions can contain one or more sphingolipid metabolite mimetics represented by Formula I wherein X is selected from the group consisting of hydrogen, methyl, alkyl, or substituted alkyl; R₁ is alkyl or substituted alkyl, preferably a linear alkyl, more preferably a C₁₀-C₂₀ alkyl; and R₂ is selected from the group consisting of phenyl, 2-furanyl, 2-thiophenyl, 2,4-heaxdienyl, 3-phenyl acrylic, isoquinoline, indole, methyl indole, and benzofuranyl.

The compositions may contains one or more sphingolipid metabolite mimetic represented by Formula II wherein Y is selected from the group consisting of hydrogen, methyl, alkyl, or substituted alkyl; R₃ is alkyl or substituted alkyl, preferably a linear alkyl, more preferably a C₁₀-C₂₀ alkyl or linear alkyl; and R₄ is selected from the group consisting of alkenyl, substituted alkenyl, aryl, or heteroaryl.

The compositions can contain one or more sphingolipid metabolite mimetics selected from the group consisting of NZJU1, NZJU2, NZJU4, NZJU10(NZJU2c), NZJU11(NZJU3c), NZJU12(NZJU4c), NZJU13(NZJU2b), NZJU14(NZJU3b), NZJU15(NZJU4b), NZJU16(NZJU2a), NZJU17(NZJU3a), NZJU18(NZJU4a), NZJU19, NZJU24(NZJU2d), NZJU25(NZJU3d), NZJU26(NZJU4d), NZJU27(NZJU2e), NZJU28(NZJU3e), NZJU29(NZJU4e), NZJU30(NZJU2f), NZJU31(NZJU3f), NZJU32(NZJU4f), NZJU33(NZJU2g), NZJU34(NZJU3g), NZJU35(NZJU4g), NZJU36(NZJU2h), NZJU37(NZJU3h), NZJU38(NZJU4h), NZJU39(NZJU1a), NZJU40(NZJU1b), NZJU41(NZJU1c), NZJU42(NZJU1d), NZJU43(NZJU1f), NZJU44(NZJU1g), NZJU45(NZJU1h), NZJU46, NZJU47(NZJU2i), NZJU48(NZJU3i), NZJU49(NZJU4i), NZJU50(NZJU2j), NZJU51(NZJU3j), NZJU52(NZJU4j), NZJU53, NZJU54, NZJU55, NZJU56, NZJU57, NZJU58, NZJU59, NZJU60, NZJU61, NZJU62, NZJU63, NZJU64. In certain embodiments the compositions contain one or more sphingolipid metabolite mimetics described herein as NZJU13(NZJU2b), NZJU16(NZJU2a), NZJU26 (NZJU4d), NZJU27(NZJU2e), NZJU28(NZJU3e), NZJU29(NZJU4e), NZJU30(NZJU2f), NZJU33(NZJU2g), NZJU34(NZJU3g), NZJU36(NZJU2h), NZJU37(NZJU3h), NZJU39(NZJU1a), NZJU41(NZJU1c), NZJU44(NZJU1g), NZJU45(NZJU1h), NZJU46, NZJU47(NZJU2i), NZJU48(NZJU3i), NZJU51(NZJU3j), NZJU57, NZJU60, NZJU62. In one embodiment the compositions contain one or more of the compounds described herein as NZJU13(NZJU2b), NZJU16(NZJU2a), NZJU30(NZJU2f), NZJU34(NZJU3g), NZJU41(NZJU1c), NZJU44(NZJU1g), and NZJU45(NZJU1h).

The compositions can be a racemic mixture of all stereoisomers of the sphingolipid metabolite mimetic or a purified mixture of only 1, or only 2, or only 3, or only 4 specific stereoisomers. The term purified refers to a mixture that is 90, 95, 99, or 99.9 percent of one isomer relative to another. The composition can contain only a single stereoisomer of one or more sphingolipid metabolite mimetics.

B. Death Receptor Agonists

The sphingolipid metabolite mimetics sensitize cells to death receptor-induced apoptosis, and compositions containing one or more sphingolipid metabolite mimetics can be administered in combination or alternation with apoptosis-inducing agents including death receptor agonists. A composition containing NZJU1, NZJU2, NZJU4, NZJU10(NZJU2c), NZJU11(NZJU3c), NZJU12(NZJU4c), NZJU13(NZJU2b), NZJU14(NZJU3b), NZJU15(NZJU4b), NZJU16(NZJU2a), NZJU17(NZJU3a), NZJU18(NZJU4a), NZJU19, NZJU24(NZJU2d), NZJU25(NZJU3d), NZJU26(NZJU4d), NZJU27(NZJU2e), NZJU28(NZJU3e), NZJU29(NZJU4e), NZJU30(NZJU2f), NZJU31(NZJU3f), NZJU32(NZJU4f), NZJU33(NZJU2g), NZJU34(NZJU3g), NZJU35(NZJU4g), NZJU36(NZJU2h), NZJU37(NZJU3h), NZJU38(NZJU4h), NZJU39(NZJU1a), NZJU40(NZJU1b), NZJU41(NZJU1c), NZJU42(NZJU1d), NZJU43(NZJU1f), NZJU44(NZJU1g), NZJU45(NZJU1h), NZJU46, NZJU47(NZJU2i), NZJU48(NZJU3i), NZJU49(NZJU4i), NZJU50(NZJU2j), NZJU51(NZJU3j), NZJU52(NZJU4j), NZJU53, NZJU54, NZJU55, NZJU56, NZJU57, NZJU58, NZJU59, NZJU60, NZJU61, NZJU62, NZJU63, NZJU64 or combinations thereof can be co-administered with a death receptor agonist. In one embodiment a composition containing one or more sphingolipid metabolite mimetics described herein as NZJU13(NZJU2b), NZJU16(NZJU2a), NZJU26 (NZJU4d), NZJU27(NZJU2e), NZJU28(NZJU3e), NZJU29(NZJU4e), NZJU30(NZJU2f), NZJU33(NZJU2g), NZJU34(NZJU3g), NZJU36(NZJU2h), NZJU37(NZJU3h), NZJU39(NZJU1a), NZJU41(NZJU1c), NZJU44(NZJU1g), NZJU45(NZJU1h), NZJU46, NZJU47(NZJU2i), NZJU48(NZJU3i), NZJU51(NZJU3j), NZJU57, NZJU60, NZJU62 is co-administered with a death receptor agonist. In another embodiment the compositions contain one or more of the compounds described herein as NZJU13(NZJU2b), NZJU16(NZJU2a), NZJU30(NZJU2f), NZJU34(NZJU3g), NZJU41(NZJU1c), NZJU44(NZJU1g), and NZJU45(NZJU1h).

Suitable death receptors include, but are not limited to, TNFR1, Fas, DR3, DR4, DR5, DR6, LTβR and combinations thereof. The death receptor agonist can be a natural ligand of a death receptor, including fragments or variants of the natural ligand that induce apoptosis. The death receptor agonist can be an antibody that binds and activates a death receptor or otherwise induces apoptosis. The death receptor agonist can be a compound, such as a small molecule identified from a compound library.

In one embodiment, the death receptor ligand is TNF-related apoptosis-inducing ligand (TRAIL). In other embodiments, the death receptor ligand is Fas ligand.

C. Additional Therapeutics

The disclosed compositions can further contain one or more additional active agents (e.g., therapeutics agents). The one or more additional active agents include antibiotics. Exemplary antibiotics include aminoglycosides, cephalosporins, chloramphenicol, clindamycin, erythromycins, fluoroquinolones, macrolides, azolides, metronidazole, penicillins, tetracyclines, trimethoprim-sulfamethoxazole, or vancomycin.

The one or more additional active can be a steroid. Exemplary steroids include but are not limited to androstanes (e.g., testosterone), cholestanes (e.g., cholesterol), cholic acids (e.g., cholic acid), corticosteroids (such as dexamethasone and prednisone), estranes (e.g., estradiol), or pregnanes (e.g., progesterone).

The composition can contain one or more classes of narcotic and non-narcotic analgesics, such as morphine, codeine, heroin, hydromorphone, levorphanol, meperidine, methadone, oxydone, propoxyphene, fentanyl, naloxone, buprenorphine, butorphanol, nalbuphine, or pentazocine.

The composition can contain one or more classes of anti-inflammatory agents, including, but not limited to salicylates (such as acetylsalicylic acid, diflunisal and salsalate), propionic acid derivatives (such as ibuprofen, naproxen, fenoprofen, ketoprofen, flurbiprofen, oxaprozin, andioxoprofen), acetic acid derivatives (such as indomethacin, sulindac, etodolac, and ketorolac), enolic acid (oxicam) derivatives (such as piroxicam, meloxicam, tenoxicam, droxicam, lornoxicam, and isoxicam), fenamic acid derivatives (such as mefenamic acid, meclofenamic acid, flufenamic acid, and tolfenamic acid), selective COX-2 inhibitors, sulphonanilides (such as nimesulide), and COX/LOX inhibitors (such as licofelone)

The composition can contain one or more classes of anti-histaminic agents, such as ethanolamines (e.g., diphenhydramine carbinoxamine), ethylenediamines (e.g., tripelennamine pyrilamine), alkylamines (e.g., chlorpheniramine, dexchlorpheniramine, brompheniramine, triprolidine), or other anti-histamines such as astemizole, loratadine, fexofenadine, bropheniramine, clemastine, acetaminophen, pseudoephedrine, and triprolidine.

The disclosed compositions can be administered alone or in combination with one or more additional therapeutic agents. For example, the disclosed compositions can be administered with an antibody or antigen binding fragment thereof specific for a growth factor receptors or tumor specific antigens. Representative growth factors receptors include, but are not limited to, epidermal growth factor receptor (EGFR; HER1); c-erbB2 (HER2); c-erbB3 (HER3); c-erbB4 (HER4); insulin receptor; insulin-like growth factor receptor 1 (IGF-1R); insulin-like growth factor receptor 2/Mannose-6-phosphate receptor (IGF-II R/M-6-P receptor); insulin receptor related kinase (IRRK); platelet-derived growth factor receptor (PDGFR); colony-stimulating factor-1receptor (CSF-1R) (c-Fms); steel receptor (c-Kit); Flk2/Flt3; fibroblast growth factor receptor 1 (Flg/Cek1); fibroblast growth factor receptor 2 (Bek/Cek3/K-Sam); Fibroblast growth factor receptor 3; Fibroblast growth factor eceptor 4; nerve growth factor receptor (NGFR) (TrkA); BDNF receptor (TrkB); NT-3-receptor (TrkC); vascular endothelial growth factor receptor 1 (Flt1); vascular endothelial growth factor receptor 2/Flk1/KDR; hepatocyte growth factor receptor (HGF-R/Met); Eph; Eck; Eek; Cek4/Mek4/HEK; Cek5; Elk/Cek6; Cek7; Sek/Cek8; Cek9; Cek10; HEK11; 9 Ror1; Ror2; Ret; Axl; RYK; DDR; and Tie.

Additional active agents include conventional cancer therapeutics such as chemotherapeutic agents, cytokines, chemokines, and radiation therapy. The majority of chemotherapeutic drugs can be divided in to: alkylating agents, antimetabolites, anthracyclines, plant alkaloids, topoisomerase inhibitors, and other antitumour agents. All of these drugs affect cell division or DNA synthesis and function in some way. Additional therapeutics include monoclonal antibodies and the new tyrosine kinase inhibitors e.g. imatinib mesylate (GLEEVEC® or GLIVEC®), which directly targets a molecular abnormality in certain types of cancer (chronic myelogenous leukemia, gastrointestinal stromal tumors).

Representative chemotherapeutic agents include, but are not limited to cisplatin, carboplatin, oxaliplatin, mechlorethamine, cyclophosphamide, chlorambucil, vincristine, vinblastine, vinorelbine, vindesine, taxol and derivatives thereof, irinotecan, topotecan, amsacrine, etoposide, etoposide phosphate, teniposide, epipodophyllotoxins, trastuzumab (HERCEPTIN®), cetuximab, and rituximab (RITUXAN® or MABTHERA®), bevacizumab (AVASTIN®), and combinations thereof.

D. Pharmaceutical Formulations

The disclosed compositions can be formulated into suitable pharmaceutical preparations such as solutions, suspensions, tablets, dispersible tablets, pills, capsules, powders, delayed and/or sustained release formulations, or elixirs for oral administration, or in sterile solutions or suspensions for parenteral administration. In one embodiment, one or more sphingolipid metabolite mimetics are formulated into pharmaceutical compositions using techniques and procedures well known in the art. In some formulations, NZJU1, NZJU2, NZJU4, NZJU10(NZJU2c), NZJU11(NZJU3c), NZJU12(NZJU4c), NZJU13(NZJU2b), NZJU14(NZJU3b), NZJU15(NZJU4b), NZJU16(NZJU2a), NZJU17(NZJU3a), NZJU18(NZJU4a), NZJU19, NZJU24(NZJU2d), NZJU25(NZJU3d), NZJU26(NZJU4d), NZJU27(NZJU2e), NZJU28(NZJU3e), NZJU29(NZJU4e), NZJU30(NZJU2f), NZJU31(NZJU3f), NZJU32(NZJU4f), NZJU33(NZJU2g), NZJU34(NZJU3g), NZJU35(NZJU4g), NZJU36(NZJU2h), NZJU37(NZJU3h), NZJU38(NZJU4h), NZJU39(NZJU1a), NZJU40(NZJU1b), NZJU41(NZJU1c), NZJU42(NZJU1d), NZJU43(NZJU1f), NZJU44(NZJU1g), NZJU45(NZJU1h), NZJU46, NZJU47(NZJU2i), NZJU48(NZJU3i), NZJU49(NZJU4i), NZJU50(NZJU2j), NZJU51(NZJU3j), NZJU52(NZJU4j), NZJU53, NZJU54, NZJU55, NZJU56, NZJU57, NZJU58, NZJU59, NZJU60, NZJU61, NZJU62, NZJU63, NZJU64 is formulated into pharmaceutical compositions.

In some embodiments, the disclosed compositions are formulated for single dosage administration. To formulate a composition, the weight fraction of active agent(s) is dissolved, suspended, dispersed or otherwise mixed in a selected carrier at an effective concentration such that the treated condition is relieved or one or more symptoms are ameliorated. The disclosed active agent(s) can be included in the pharmaceutically acceptable carrier in an amount sufficient to exert a therapeutically useful effect in the absence of undesirable side effects on the patient treated. The therapeutically effective concentration is determined empirically by testing the compounds in in vitro, ex vivo and in vivo systems, and then extrapolated therefrom for dosages for humans. The concentration of active agent(s) in the pharmaceutical composition will depend on absorption, inactivation and excretion rates of the active compound, the physicochemical characteristics of the agent, the dosage schedule, and amount administered as well as other factors known to those of skill in the art.

Dosage forms or compositions containing active agent(s) in the range of 0.005% to 100% with the balance made up from non-toxic carrier may be prepared. Methods for preparation of these compositions are known to those skilled in the art. The contemplated compositions may contain 0.001%-100% active ingredient, or in one embodiment 0.1-95%.

Methods for solubilizing active agents or improving bioavailability may be used. Such methods are known to those of skill in this art, and include, but are not limited to, using cosolvents, such as dimethylsulfoxide (DMSO), using surfactants, such as TWEEN®, or dissolution in aqueous sodium bicarbonate. The pharmaceutical compositions of one or more of the active agents can be incorporated into a polymer matrix, for example, hydroxypropylmethyl cellulose, gel, permeable membrane, osmotic system, multilayer coating, microparticle, nanoparticle, liposome, microsphere, nanosphere, or the like. The active agent(s) may be suspended in micronized or other suitable form or may be derivatized (e.g., by adding one or more polyethylene glycol chains) to produce a more soluble active product or improve bioavailability. To optimize absorption, distribution, metabolism, and excretion, or improve oral bioavailability, the active agent(s) may be provided as prodrugs (i.e. in an inactive or significantly less active form which is metabolised in vivo into an active agent). The active agent(s) described herein may also be conjugated to a biomolecule, including, but not limited to a protein or nucleic acid, to affect bioavailability.

Liquid pharmaceutically administrable compositions can, for example, be prepared by dissolving, dispersing, or otherwise mixing the active agent(s) and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrin derivatives, sorbitan monolaurate, triethanolamine sodium acetate, triethanolamine oleate, and other such agents.

Oral pharmaceutical dosage forms can be either solid, gel, or liquid. The solid dosage forms can be tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating.

The active agent(s), or a pharmaceutically acceptable salt(s) thereof, can be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes, and cellulose acetate phthalate.

The pharmaceutical composition can be in a parenteral administration form. Injectables can be prepared in conventional forms, either as liquid solutions or suspensions, solid forms suitable for solution or suspension in liquid prior to injection, or as emulsions. The injectables, solutions, and emulsions may also contain one or more excipients. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. The percentage of active compound contained in such parenteral compositions is highly dependent on the specific nature thereof, as well as the activity of the compound and the needs of the subject.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

The unit-dose parenteral preparations can be packaged in an ampoule, a vial or a syringe with a needle. All preparations for parenteral administration should be sterile, as is known and practiced in the art. The injectable compositions described herein can be optimized for local and/or systemic administration.

The active agent(s) may be suspended in micronized or other suitable form. The active agent(s) may also be derivatized to produce a more soluble active product or to produce a prodrug. The form of the resulting mixture depends upon a number of factors, including the intended mode of administration and the solubility of the active agent(s) in the selected carrier or vehicle. The effective concentration is sufficient for ameliorating the symptoms of the condition and may be empirically determined.

Implantation of a slow-release or sustained-release system, such that a constant level of dosage is maintained is also contemplated herein. In such cases, the active agent(s) provided herein can be dispersed in a solid matrix optionally coated with an outer rate-controlling membrane. The compound diffuses from the solid matrix (and optionally through the outer membrane) sustained, rate-controlled release. The solid matrix and membrane may be formed from any suitable material known in the art including, but not limited to, polymers, bioerodible polymers, and hydrogels.

Lyophilized powders can be reconstituted for administration as solutions, emulsions and other mixtures. They may also be reconstituted and formulated as solids or gels. The sterile, lyophilized powder can be prepared by dissolving a disclosed active agent, such as NZJU1, NZJU2, NZJU4, NZJU10(NZJU2c), NZJU11(NZJU3c), NZJU12(NZJU4c), NZJU13(NZJU2b), NZJU14(NZJU3b), NZJU15(NZJU4b), NZJU16(NZJU2a), NZJU17(NZJU3a), NZJU18(NZJU4a), NZJU19, NZJU24(NZJU2d), NZJU25(NZJU3d), NZJU26(NZJU4d), NZJU27(NZJU2e), NZJU28(NZJU3e), NZJU29(NZJU4e), NZJU30(NZJU2f), NZJU31(NZJU3f), NZJU32(NZJU4f), NZJU33(NZJU2g), NZJU34(NZJU3g), NZJU35(NZJU4g), NZJU36(NZJU2h), NZJU37(NZJU3h), NZJU38(NZJU4h), NZJU39(NZJU1a), NZJU40(NZJU1b), NZJU41(NZJU1c), NZJU42(NZJU1d), NZJU43(NZJU1f), NZJU44(NZJU1g), NZJU45(NZJU1h), NZJU46, NZJU47(NZJU2i), NZJU48(NZJU3i), NZJU49(NZJU4i), NZJU50(NZJU2j), NZJU51(NZJU3j), NZJU52(NZJU4j), NZJU53, NZJU54, NZJU55, NZJU56, NZJU57, NZJU58, NZJU59, NZJU60, NZJU61, NZJU62, NZJU63, NZJU64, or a pharmaceutically acceptable salt thereof, in a suitable solvent. The solvent may contain an excipient which improves the stability or other pharmacological component of the powder or reconstituted solution, prepared from the powder. The solvent may also contain a buffer, such as citrate, sodium or potassium phosphate or other such buffer known to those of skill in the art at. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

The disclosed active agent(s), or pharmaceutically acceptable salts thereof, can be formulated to be targeted to a particular tissue, receptor, or other area of the body of the subject to be treated. Many such targeting methods are well known to those of skill in the art. In one embodiment, liposomal suspensions, including tissue-targeted liposomes, such as tumor-targeted liposomes, may also be suitable as pharmaceutically acceptable carriers. These may be prepared according to methods known to those skilled in the art.

Compositions can be formulated to provide immediate or delayed release of one or more of the active agent(s), including sphingolipid metabolite mimetics. Also disclosed are sustained release formulations to maintain therapeutically effective amounts of one or more active agents, including sphingolipid metabolite mimetics, over a period of time. In some embodiments, the sphingolipid metabolite mimetic is NZJU1, NZJU2, NZJU4, NZJU10(NZJU2c), NZJU11(NZJU3c), NZJU12(NZJU4c), NZJU13(NZJU2b), NZJU14(NZJU3b), NZJU15(NZJU4b), NZJU16(NZJU2a), NZJU17(NZJU3a), NZJU18(NZJU4a), NZJU19, NZJU24(NZJU2d), NZJU25(NZJU3d), NZJU26(NZJU4d), NZJU27(NZJU2e), NZJU28(NZJU3e), NZJU29(NZJU4e), NZJU30(NZJU2f), NZJU31(NZJU3f), NZJU32(NZJU4f), NZJU33(NZJU2g), NZJU34(NZJU3g), NZJU35(NZJU4g), NZJU36(NZJU2h), NZJU37(NZJU3h), NZJU38(NZJU4h), NZJU39(NZJU1a), NZJU40(NZJU1b), NZJU41(NZJU1c), NZJU42(NZJU1d), NZJU43(NZJU1f), NZJU44(NZJU1g), NZJU45(NZJU1h), NZJU46, NZJU47(NZJU2i), NZJU48(NZJU3i), NZJU49(NZJU4i), NZJU50(NZJU2j), NZJU51(NZJU3j), NZJU52(NZJU4j), NZJU53, NZJU54, NZJU55, NZJU56, NZJU57, NZJU58, NZJU59, NZJU60, NZJU61, NZJU62, NZJU63, NZJU64.

In compositions containing multiple active agents, the active agents may be individually formulated to control the duration and/or time release of each active agent. In one embodiment, a composition containing one or more sphingolipid metabolite mimetics further contains a death receptor agonist formulated for sustained and/or timed release. In one embodiment, a composition containing one or more sphingolipid metabolite mimetics further contains an anti-neoplastic agent formulated for sustained and/or timed release.

Such sustained and/or timed release formulations may be made by sustained release means of delivery devices that are well known to those of ordinary skill in the art. These pharmaceutical compositions can be used to provide slow or sustained release of one or more of the active agents using, for example, hydroxypropylmethyl cellulose, other polymer matrices, gels, permeable membranes, osmotic systems, multilayer coatings, microparticles, nanoparticles, liposomes, microspheres, nanospheres or the like. The active agents may also be suspended, micronized, or derivatized to vary release of the active ingredient(s).

In some embodiments, the composition containing one or more sphingolipid metabolite mimetics is provided in a kit, optionally containing a composition containing a death receptor agonist. In one embodiment, the composition containing NZJU1, NZJU2, NZJU4, NZJU10(NZJU2c), NZJU11(NZJU3c), NZJU12(NZJU4c), NZJU13(NZJU2b), NZJU14(NZJU3b), NZJU15(NZJU4b), NZJU16(NZJU2a), NZJU17(NZJU3a), NZJU18(NZJU4a), NZJU19, NZJU24(NZJU2d), NZJU25(NZJU3d), NZJU26(NZJU4d), NZJU27(NZJU2e), NZJU28(NZJU3e), NZJU29(NZJU4e), NZJU30(NZJU2f), NZJU31(NZJU3f), NZJU32(NZJU4f), NZJU33(NZJU2g), NZJU34(NZJU3g), NZJU35(NZJU4g), NZJU36(NZJU2h), NZJU37(NZJU3h), NZJU38(NZJU4h), NZJU39(NZJU1a), NZJU40(NZJU1b), NZJU41(NZJU1c), NZJU42(NZJU1d), NZJU43(NZJU1f), NZJU44(NZJU1g), NZJU45(NZJU1h), NZJU46, NZJU47(NZJU2i), NZJU48(NZJU3i), NZJU49(NZJU4i), NZJU50(NZJU2j), NZJU51(NZJU3j), NZJU52(NZJU4j), NZJU53, NZJU54, NZJU55, NZJU56, NZJU57, NZJU58, NZJU59, NZJU60, NZJU61, NZJU62, NZJU63, NZJU64 is provided in a kit, optionally containing a composition containing a death receptor agonist.

### III. Methods of Use

A. Treating Cancer and Other Proliferative Diseases

Methods and compositions are provided for use in the treatment of diseases associated with inappropriate survival or proliferation of target cells, including those attributable to dysregulation of the apoptosis systems in cancer or in inflammatory and autoimmune diseases.

Inflammatory diseases are primarily T cell-mediated (Mottet et al. Journal of Immunology, 2003, 170:3939-3943; Ostanin et al. American Journal of Physiology—Gastrointestinal and Liver Physiology, 2009, 296:135-146; Uronis et al. PLoS One, 2009, 4, e6026). Accumulation of hyperactivated T cells causes colonic inflammation that often leads to inflammatory diseases and autoimmune disease (Arthur and Jobin Inflammatory Bowell Diseases, 2011, 17:396-409; Arthur et al. Science, 2012; Leonardo et al. Immunity, 2010, 32:291-295; Mottet et al. Journal of Immunology, 2003, 170:3939-3943; Ostanin et al. American Journal of Physiology—Gastrointestinal and Liver Physiology, 2009, 296:135-146; Nikolov et al. Blood, 2010, 116:740-747; Ten Hove et al. Journal of Leukocyte Biology, 2004, 75:1010-1015). Termination of an immune response after clearance of pathogen-infected or diseased cells is by elimination of effector T cells through Fas-FasL interaction to induce Fas-mediated apoptosis under physiological conditions. T cell homeostasis, including colonic T cell hemostasis, is controlled by Fas-mediated apoptosis (De Maria et al. Journal of Clinical Investigations, 1996, 97:316-322; Russel and Ley Annual Reviews in Immunology, 2002, 20:323-370) T cells, especially activated T cells, often express high levels of Fas under normal conditions (De Maria et al. Journal of Clinical Investigations, 1996, 97:316-322; Hongo et al. Journal of International Medical Research, 2000, 28:132-142; however, in cases of inflammatory disease, such as ulcerative colitis, Fas expression levels in colonic T cells as well as colonic T cell sensitivity to Fas-mediated apoptosis is often decreased (Bu et al. Journal of Immunology, 2001, 166:6399-6403; Suzuki et al. Scandinavian Journal of Gastroenterology, 2000, 45:1278-1283), suggesting that down-regulation of Fas receptor expression and decreased sensitivity to Fas-mediated apoptosis is responsible, at least in part, for T cell persistence in the colonic mucosa under inflammatory conditions. Sphingosine metabolites, particularly ceramide, have been shown to mediate T cell apoptosis (Cremesti et al. Journal of Biological Chemistry, 2001, 276:23954-23961). Therefore, sphingolipid mimetics have the potential to be developed as therapeutic agents for inflammatory and autoimmune disease. Inflammatory and autoimmune diseases illustratively include systemic lupus erythematosus, Hashimoto's disease, rheumatoid arthritis, graft-versus-host disease, Sjogren's syndrome, pernicious anemia, Addison disease, scleroderma, Goodpasture's syndrome, Crohn's disease, autoimmune hemolytic anemia, sterility, myasthenia gravis, multiple sclerosis, Basedow's disease, thrombopenia purpura, insulin-dependent diabetes mellitus, allergy, asthma, atopic disease, arteriosclerosis, myocarditis, cardiomyopathy, glomerular nephritis, hypoplastic anemia, rejection after organ transplantation.

Cancers which can be treated using the composition and methods describe herein include sarcomas, lymphomas, leukemias, carcinomas, blastomas, and germ cell tumors. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

A method for treating a subject with cancer is provided. This method involves administering to the subject a composition containing one or more sphingolipid metabolite mimetics. In some embodiments, the sphingolipid metabolite mimetic is NZJU1, NZJU2, NZJU4, NZJU10(NZJU2c), NZJU11(NZJU3c), NZJU12(NZJU4c), NZJU13(NZJU2b), NZJU14(NZJU3b), NZJU15(NZJU4b), NZJU16(NZJU2a), NZJU17(NZJU3a), NZJU18(NZJU4a), NZJU19, NZJU24(NZJU2d), NZJU25(NZJU3d), NZJU26(NZJU4d), NZJU27(NZJU2e), NZJU28(NZJU3e), NZJU29(NZJU4e), NZJU30(NZJU2f), NZJU31(NZJU3f), NZJU32(NZJU4f), NZJU33(NZJU2g), NZJU34(NZJU3g), NZJU35(NZJU4g), NZJU36(NZJU2h), NZJU37(NZJU3h), NZJU38(NZJU4h), NZJU39(NZJU1a), NZJU40(NZJU1b), NZJU41(NZJU1c), NZJU42(NZJU1d), NZJU43(NZJU1f), NZJU44(NZJU1g), NZJU45(NZJU1h), NZJU46, NZJU47(NZJU2i), NZJU48(NZJU3i), NZJU49(NZJU4i), NZJU50(NZJU2j), NZJU51(NZJU3j), NZJU52(NZJU4j), NZJU53, NZJU54, NZJU55, NZJU56, NZJU57, NZJU58, NZJU59, NZJU60, NZJU61, NZJU62, NZJU63, NZJU64. Preferably the sphingolipid metabolite mimetic used for treating a subject with cancer is NZJU13(NZJU2b), NZJU16(NZJU2a), NZJU26(NZJU4d), NZJU27(NZJU2e), NZJU28(NZJU3e), NZJU29(NZJU4e), NZJU30(NZJU2f), NZJU33(NZJU2g), NZJU34(NZJU3g), NZJU36(NZJU2h), NZJU37(NZJU3h), NZJU39(NZJU1a), NZJU41(NZJU1c), NZJU44(NZJU1g), NZJU45(NZJU1h), NZJU46, NZJU47(NZJU2i), NZJU48(NZJU3i), NZJU51(NZJU3j), NZJU57, NZJU60, NZJU62. In particularly preferred embodiments it is NZJU13(NZJU2b), NZJU16(NZJU2a), NZJU30(NZJU2f), NZJU34(NZJU3g), NZJU41(NZJU1c), NZJU44(NZJU1g), and NZJU45(NZJU1h).

It has been shown, for example, that NZJU2i and NZJU3i have an IC50 of less than 20 μM for multiple types of cancer cells. Therefore, in some embodiments, the composition can contain about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 450, 500, 550, 600, 650, 700, 750, 800, 850, 900, 950, or 1000 μM of one or more sphingolipid metabolite mimetics described by Formulas I. In some embodiments, the sphingolipid metabolite mimetic is NZJU1, NZJU2, NZJU4, NZJU10(NZJU2c), NZJU11(NZJU3c), NZJU12(NZJU4c), NZJU13(NZJU2b), NZJU14(NZJU3b), NZJU15(NZJU4b), NZJU16(NZJU2a), NZJU17(NZJU3a), NZJU18(NZJU4a), NZJU19, NZJU24(NZJU2d), NZJU25(NZJU3d), NZJU26(NZJU4d), NZJU27(NZJU2e), NZJU28(NZJU3e), NZJU29(NZJU4e), NZJU30(NZJU2f), NZJU31(NZJU3f), NZJU32(NZJU4f), NZJU33(NZJU2g), NZJU34(NZJU3g), NZJU35(NZJU4g), NZJU36(NZJU2h), NZJU37(NZJU3h), NZJU38(NZJU4h), NZJU39(NZJU1a), NZJU40(NZJU1b), NZJU41(NZJU1c), NZJU42(NZJU1d), NZJU43(NZJU1f), NZJU44(NZJU1g), NZJU45(NZJU1h), NZJU46, NZJU47(NZJU2i), NZJU48(NZJU3i), NZJU49(NZJU4i), NZJU50(NZJU2j), NZJU51(NZJU3j), NZJU52(NZJU4j), NZJU53, NZJU54, NZJU55, NZJU56, NZJU57, NZJU58, NZJU59, NZJU60, NZJU61, NZJU62, NZJU63, NZJU64.

In preferred embodiments, a therapeutic amount of composition containing one or more sphingolipid metabolite mimetics is co-administered with a therapeutic amount of a composition containing death receptor agonist, wherein the sphingolipid metabolite mimetic(s) reduce resistance of the cancer cells to the death receptor agonist. In some embodiments, the sphingolipid metabolite mimetic is NZJU1, NZJU2, NZJU4, NZJU10(NZJU2c), NZJU11(NZJU3c), NZJU12(NZJU4c), NZJU13(NZJU2b), NZJU14(NZJU3b), NZJU15(NZJU4b), NZJU16(NZJU2a), NZJU17(NZJU3a), NZJU18(NZJU4a), NZJU19, NZJU24(NZJU2d), NZJU25(NZJU3d), NZJU26(NZJU4d), NZJU27(NZJU2e), NZJU28(NZJU3e), NZJU29(NZJU4e), NZJU30(NZJU2f), NZJU31(NZJU3f), NZJU32(NZJU4f), NZJU33(NZJU2g), NZJU34(NZJU3g), NZJU35(NZJU4g), NZJU36(NZJU2h), NZJU37(NZJU3h), NZJU38(NZJU4h), NZJU39(NZJU1a), NZJU40(NZJU1b), NZJU41(NZJU1c), NZJU42(NZJU1d), NZJU43(NZJU1f), NZJU44(NZJU1g), NZJU45(NZJU1h), NZJU46, NZJU47(NZJU2i), NZJU48(NZJU3i), NZJU49(NZJU4i), NZJU50(NZJU2j), NZJU51(NZJU3j), NZJU52(NZJU4j), NZJU53, NZJU54, NZJU55, NZJU56, NZJU57, NZJU58, NZJU59, NZJU60, NZJU61, NZJU62, NZJU63, NZJU64.

Sphingolipid metabolite mimetics enhance the efficacy of TRAIL in TRAIL induced apoptosis. Therefore, a therapeutic amount of composition containing one or more sphingolipid metabolite mimetics can be given in combination with TRAIL. The co-administration of one or more sphingolipid metabolite mimetics and death receptor agonists can be simultaneous or sequential. Simultaneous administration includes the use of a single composition containing both sphingolipid metabolite mimetics and a death receptor agonist. Simultaneous administration also includes administration of separate compositions of one or more sphingolipid metabolite mimetics and a death receptor agonist at substantially the same time. “Substantially the same time” includes administration of the second composition within 1 minute of the first composition.

In some embodiments, the compositions are administered sequentially. In this method, the composition containing one or more sphingolipid metabolite mimetics is administered first to sensitize the target cells prior to administration of the second composition containing the death receptor agonist. For example, the composition containing one or more sphingolipid metabolite mimetics can be administered from 1 minute to 7 days before administration of the composition containing the death receptor agonist. For example, the composition containing one or more sphingolipid metabolite mimetics can be administered at least about 1, 2, 3, 4, 5, 6, 7, 8, 9, 10, 11, 12, 13, 14, 15, 16, 17, 18, 19, 20, 25, 30, 35, 40, 45, 50, 60 minutes before administration of the composition containing the death receptor agonist. For example, the composition containing one or more sphingolipid metabolite mimetics can be administered at least about 1, 2, 3, 4, 5, 6, 7, 8, 9, 10, 11, 12, 13, 14, 15, 16, 17, 18, 19, 20, 21, 22, 23, 24 hours before administration of the composition containing the death receptor agonist. For example, the composition containing one or more sphingolipid metabolite mimetics can be administered at least about 1, 2, 3, 4, 5, 6, 7 days before administration of the composition containing the death receptor agonist.

Sequential administration can also be accomplished by administering a composition containing one or more sphingolipid metabolite mimetics and a delayed release formulation of the death receptor agonist.

In one method, a composition containing one or more sphingolipid metabolite mimetics is administered locally to sensitize the target cells while a composition containing a death receptor agonist is administered systemically. In one method, a composition containing one or more sphingolipid metabolite mimetics is administered systemically to sensitize the target cells while a composition containing a death receptor agonist is administered locally.

The cancer of the disclosed methods can be any cell in a subject undergoing unregulated growth, invasion, or metastasis. In some aspects, the cancer can be resistant to TRAIL-induced apoptosis.

B. Sensitizing Cells to Death Receptor Agonists

A method of selectively inducing apoptosis in a target cell expressing a death receptor is provided. The method generally involves contacting the cell with a composition containing an effective amount of one or more sphingolipid metabolite mimetics and a death receptor agonist that binds the death receptor on the target cell. In some embodiments, the method involves contacting the cell with NZJU1, NZJU2, NZJU4, NZJU10(NZJU2c), NZJU11(NZJU3c), NZJU12(NZJU4c), NZJU13(NZJU2b), NZJU14(NZJU3b), NZJU15(NZJU4b), NZJU16(NZJU2a), NZJU17(NZJU3a), NZJU18(NZJU4a), NZJU19, NZJU24(NZJU2d), NZJU25(NZJU3d), NZJU26(NZJU4d), NZJU27(NZJU2e), NZJU28(NZJU3e), NZJU29(NZJU4e), NZJU30(NZJU2f), NZJU31(NZJU3f), NZJU32(NZJU4f), NZJU33(NZJU2g), NZJU34(NZJU3g), NZJU35(NZJU4g), NZJU36(NZJU2h), NZJU37(NZJU3h), NZJU38(NZJU4h), NZJU39(NZJU1a), NZJU40(NZJU1b), NZJU41(NZJU1c), NZJU42(NZJU1d), NZJU43(NZJU1f), NZJU44(NZJU1g), NZJU45(NZJU1h), NZJU46, NZJU47(NZJU2i), NZJU48(NZJU3i), NZJU49(NZJU4i), NZJU50(NZJU2j), NZJU51(NZJU3j), NZJU52(NZJU4j), NZJU53, NZJU54, NZJU55, NZJU56, NZJU57, NZJU58, NZJU59, NZJU60, NZJU61, NZJU62, NZJU63, or NZJU64 and a death receptor agonist that binds the death receptor on the target cell. In all such embodiments, the sphingolipid metabolite mimetics and the death receptor agonist can be in the same composition or in separate compositions.

It has been shown that concentrations of sphingolipid metabolite mimetics between 20 and 100 μM can be effective for various cancer cell lines to overcome TRAIL resistance. In some embodiments, the method involves contacting the cell with one or more sphingolipid metabolite mimetics at a concentration of about 10 to 200 μM sphingolipid metabolite mimetic, including about 20 to 100 μM sphingolipid metabolite mimetic. In some methods, cells are contacted with one or more sphingolipid metabolite mimetics at a concentration of about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 450, 500, 550, 600, 650, 700, 750, 800, 850, 900, 950, or 1000 μM sphingolipid metabolite mimetic.

Sphingolipid metabolite mimetic sensitize the cell to death receptor-induced apopotis. In some embodiments, compositions containing sphingolipid metabolite mimetics and further containing one or more death receptor agonists increases cell death by more than 10%, 15%, 20%, 25%, 30%, 35%, 40%, 45%, 50%, 55%, 60%, 65%, 70%, 75%, 80%, 90%, or 100% as compared to administration of the death receptor agonist alone. In preferred embodiments, the cell is resistant to apoptosis induced by the death receptor agonist. Therefore, the method can in some embodiments induce apoptosis in a target cell that will not undergo apoptosis when contacted with the same amount of death receptor agonist alone. In other embodiments, the method reduces the amount of death receptor agonist required to induce apoptosis in the target cell.

C. Therapeutic Administration

The disclosed compositions, including pharmaceutical compositions, may be administered in a number of ways depending on whether local or systemic treatment is desired, and on the area to be treated. For example, the disclosed compositions can be administered orally, parenterally (e.g., intravenous, intramuscular, intraperitoneal, subcutaneous injection), topically or the like.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

The disclosed compositions may be administered prophylactically, e.g., to patients or subjects who are at risk for cancer growth or metastasis. Thus, the method can further comprise identifying a subject at risk for cancer growth or metastasis prior to administration of the disclosed compositions.

The exact amount of the compositions required will vary from subject to subject, depending on the species, age, sex, weight and general condition of the subject, extent of the disease in the subject, route of administration, whether other drugs are included in the regimen, and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. Dosage can vary, and can be administered in one or more dose administrations daily, for one or several days. Guidance can be found in the literature for appropriate dosages for given classes of pharmaceutical products.

### IV. Definitions

The term “amine-protecting group”, as used herein, refers to any chemical functional group that may be attached to a primary or secondary amine to impart stability under specified reaction conditions, preventing the formation of undesired bonds and side reactions involving the amine nitrogen. Exemplary amine-protecting groups include but are certainly not limited to benzyloxycarbonyl, 9-fluoromethyloxycarbonyl, t-butyloxycarbonyl (Boc), acetyl, trifluoroacetyl, benzyl, triphenylmethyl (Trityl), benzylideneamine, p-toluenesulfonamide, 3,5-dimethoxyphenylisoproxycarbonyl, 2-(4-biphenyl)isoproxycarbonyl, 2-nitrophenylsulfenyl, or suitable derivatives or analogs thereof. Upon seeing the mechanisms described below, one of skill in the art will envision many alternative amine-protecting groups. The amine-protecting group can be attached to and subsequently removed from the amine nitrogen using any method or reaction conditions known in the art. Optimal methods and conditions for attaching and subsequently removing the amine-protecting group will depend upon many factors, including the choice of protecting group. One of skill in the art will readily envision many methods for attaching and subsequently removing the amine-protecting group. Exemplary amine protecting groups as well as methods for attaching and subsequently removing them include those described in Isidro-Llobet et al. Chemical Reviews 2009, 109:2455-2504 and the references cited therein.

The term “mimetic”, as used herein, refers to a chemical substance that has similar structure and properties to a naturally occurring molecule, or that has similar structure to a naturally occurring molecule, or that has similar properties to a naturally occurring molecule, or that mimics the binding of a naturally occurring molecule to a receptor. A chemical substance that has a similar structure to, or that has similar properties to, or that mimics one or more binding affinities of a sphingolipid metabolite to a receptor is referred to as a “sphingolipid metabolite mimetic.” In certain embodiments the sphingolipid metabolite mimetic is a derivative or analogue of a naturally occurring sphingolipid metabolite. In certain embodiments the sphingolipid metabolite mimetic exhibits a binding affinity to one or more receptors that is less than that of the analogous naturally occurring sphingolipid metabolite. In certain embodiments the sphingolipid metabolite mimetic exhibits a binding affinity to one or more receptors that is approximately identical to that of the analogous naturally occurring sphingolipid metabolite. In certain embodiments the sphingolipid metabolite mimetic exhibits a binding affinity to one or more receptors that is greater than that of the analogous naturally occurring sphingolipid metabolite. In certain embodiments, the sphingolipid metabolite mimetic is not a derivative or analogue of a naturally occurring sphingolipid metabolite.

The term “intermediate” or “synthetic intermediate”, as used interchangeably herein, refers to a chemical substance or compound that is necessarily produced or synthesized during the synthesis of a different product, substance, or compound. The intermediate may be obtained, isolated, extracted, purified, crystallized, or separated in some manner from the reaction mixture using a number of methods known in the art. The synthetic intermediate need not necessarily be obtained, isolated, extracted, purified, crystallized, or separated in any manner from the reaction mixture. The intermediate may be produced and subsequently consumed, transformed, derivatized, or reacted with to produce the desired product or to produce a different intermediate without ever obtaining, isolating, extracting, purifying, crystallizing, or separating the intermediate from the reaction mixture.

The term “co-administration”, as used herein, includes simultaneous and sequential administration. An appropriate time course for sequential administration may be chosen by the physician, according to such factors as the nature of a patient's illness, and the patient's condition.

The term “death receptor”, as used herein, refers to a cell-surface receptor that induces cellular apoptosis once bound by a ligand. Death receptors preferably include tumor necrosis factor (TNF) receptor superfamily members having death domains (e.g., TNFR1, Fas, DR3, DR4, DR5, DR6, and LTβR).

The term “death receptor agonist”, as used herein, refers to a substance that is capable of binding a death receptor on a cell and initiating apoptosis. For example, a “death receptor agonist small molecule” is a compound that is capable of interacting with the death receptor to initiate apoptosis.

The terms “inhibit,” “inhibiting,” or “inhibition”, as used herein, refer to a decrease in activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “subject”, as used herein, refers to any individual who is the target of administration. The subject can be a vertebrate, for example, a mammal. Thus, the subject can be a human. The term does not denote a particular age or sex. The term “patient” refers to a subject afflicted with a disease or disorder. The term “patient” includes human and veterinary subjects.

The term “target cell”, as used herein, refers to a cell bearing the targeted death receptor, including, for example, a cell that expresses DR5 or DR4. Preferably, the target cell is an abnormally growing cell or tumor cell.

The term “therapeutically effective”, as used herein, means that the amount of the composition used is of sufficient quantity to ameliorate one or more causes or symptoms of a disease or disorder. Such amelioration only requires a reduction or alteration, not necessarily elimination. For example, a therapeutically effective amount of a composition containing a death receptor agonist is the quantity sufficient to cause apoptosis in one or more target cells. As used herein, the terms “therapeutically effective amount” “therapeutic amount” and “pharmaceutically effective amount” are synonymous. One of skill in the art could readily determine the proper therapeutic amount.

The terms “treat” or “treatment”, as used herein, refer to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The terms “prevent,” “preventing,” or “prevention”, as used herein, do not require absolute forestalling of the condition or disease but can also include a reduction in the onset or severity of the disease or condition. For example, in the case of death receptor resistance, to prevent a target cell's resistance to a death receptor agonist is to make the cell less resistant to said agonist.

The terms “analog” and “derivative”, as used interchangeably herein, refer to a compound having a structure similar to that a parent compound, but varying from the parent compound by a difference in one or more certain components. The analog or derivative can differ from the parent compound in one or more atoms, functional groups, or substructures, which are replaced with other atoms, groups, or substructures. An analog or derivative can be imagined to be formed, at least theoretically, from the parent compound via some chemical or physical process. The terms analog and derivative encompass compounds which retain the same basic ring structure as the parent compound, but possess one or more different substituents on the ring(s). The terms analog and derivative also encompasses compounds which possesses a different ring structure from the parent compound which is obtained via chemical modification of the parent compound.

The term “aryl”, as used herein, refers to C₅-C₁₀-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles”, “heteroaromatics”, or “heteroaryls”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

The term “alkyl”, as used herein, refers to the radical of saturated or unsaturated aliphatic groups, including straight-chain alkyl, alkenyl, or alkynyl groups, branched-chain alkyl, alkenyl, or alkynyl groups, cycloalkyl, cycloalkenyl, or cycloalkynyl (alicyclic) groups, alkyl substituted cycloalkyl, cycloalkenyl, or cycloalkynyl groups, and cycloalkyl substituted alkyl, alkenyl, or alkynyl groups. Unless otherwise indicated, a straight chain or branched chain alkyl has 30 or fewer carbon atoms in its backbone (e.g., C₁-C₃₀ for straight chain, C₃-C₃₀ for branched chain), preferably 20 or fewer, more preferably from about 10 to 20. If the alkyl is unsaturated, the alkyl chain generally has from 2-30 carbons in the chain, preferably from 2-20 carbons in the chain, more preferably from 10-20 carbons in the chain. Likewise, preferred cycloalkyls have from 3-20 carbon atoms in their ring structure, preferably from 3-10 carbons atoms in their ring structure, most preferably 5, 6 or 7 carbons in the ring structure.

The terms “alkenyl” and “alkynyl” refer to unsaturated aliphatic groups analogous in length and possible substitution to the alkyls described above, but that contain at least one double or triple bond respectively.

The term “alkyl” includes one or more substitutions at one or more carbon atoms of the hydrocarbon radical as well as heteroalkyls. Suitable substituents include, but are not limited to, halogens, such as fluorine, chlorine, bromine, or iodine; hydroxyl; —NR₁R₂, wherein R₁ and R₂ are independently hydrogen, alkyl, or aryl, and wherein the nitrogen atom is optionally quaternized; —SR, wherein R is hydrogen, alkyl, or aryl; —CN; —NO₂; —COOH; carboxylate; —COR, —COOR, or —CONR₂, wherein R is hydrogen, alkyl, or aryl; azide, aralkyl, alkoxyl, imino, phosphonate, phosphinate, silyl, ether, sulfonyl, sulfonamido, heterocyclyl, aromatic or heteroaromatic moieties, —CF₃; —CN; —NCOCOCH₂CH₂; —NCOCOCHCH; —NCS; and combinations thereof.

The terms “amino” and “amine”, as used herein, are art-recognized and refer to both substituted and unsubstituted amines, e.g., a moiety that can be represented by the general formula:

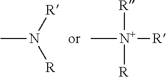

wherein, R, R′, and R″ each independently represent a hydrogen, substituted or unsubstituted alkyl, substituted or unsubstituted alkenyl, substituted or unsubstituted alkynyl, substituted or unsubstituted carbonyl, —(CH₂)ₘ—R′″, or R and R′ taken together with the N atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred embodiments, only one of R and R′ can be a carbonyl, e.g., R and R′ together with the nitrogen do not form an imide. In preferred embodiments, R and R′ (and optionally R″) each independently represent a hydrogen atom, substituted or unsubstituted alkyl, a substituted or unsubstituted alkenyl, or —(CH₂)ₘ—R′″. Thus, the term ‘alkylamine’ as used herein refers to an amine group, as defined above, having a substituted or unsubstituted alkyl attached thereto (i.e. at least one of R, R′, or R″ is an alkyl group).

The term “carbonyl”, as used herein, is art-recognized and includes such moieties as can be represented by the general formula:

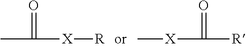

wherein X is a bond, or represents an oxygen or a sulfur, and R represents a hydrogen, a substituted or unsubstituted alkyl, substituted or unsubstituted alkenyl, substituted or unsubstituted alkynyl, —(CH₂)ₘ—R″, or a pharmaceutical acceptable salt, R′ represents a hydrogen, substituted or unsubstituted alkyl, substituted or unsubstituted alkenyl, substituted or unsubstituted alkynyl, or —(CH₂)ₘ—R″; R″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. Where X is oxygen and R is defines as above, the moiety is also referred to as a carboxyl group. When X is oxygen and R is hydrogen, the formula represents a ‘carboxylic acid’. Where X is oxygen and R′ is hydrogen, the formula represents a ‘formate’. In general, where the oxygen atom of the above formula is replaced by a sulfur, the formula represents a ‘thiocarbonyl’ group. Where X is sulfur and R or R′ is not hydrogen, the formula represents a ‘thioester’. Where X is sulfur and R is hydrogen, the formula represents a ‘thiocarboxylic acid’. Where X is sulfur and R′ is hydrogen, the formula represents a ‘thioformate’. Where X is a bond and R is not hydrogen, the above formula represents a ‘ketone’. Where X is a bond and R is hydrogen, the above formula represents an ‘aldehyde’.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized.

Examples of saturated hydrocarbon radicals include, but are not limited to, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, and 3-butynyl.

The terms “alkoxy”, “alkylamino”, and “alkylthio” are used herein in their conventional sense, and refer to those alkyl groups attached to the remainder of the molecule via an oxygen atom, an amino group, or a sulfur atom, respectively.

The term “alkylaryl”, as used herein, refers to an alkyl group substituted with an aryl group (e.g., an aromatic or hetero aromatic group).

The terms “heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, C₁-C₁₀ alkyl, phenyl or benzyl, and optionally containing 1-3 double bonds and optionally substituted with one or more substituents. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Heterocyclic groups can optionally be substituted with one or more substituents as defined above for alkyl and aryl.

The term “halogen”, as used herein, refers to fluorine, chlorine, bromine, or iodine.

The term “pharmaceutically acceptable salt”, as used herein, refers to derivatives of the compounds defined herein, wherein the parent compound is modified by making acid or base salts thereof. Example of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, naphthalenesulfonic, methanesulfonic, ethane disulfonic, oxalic, and isethionic salts.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704; and “Handbook of Pharmaceutical Salts: Properties, Selection, and Use,” P. Heinrich Stahl and Camille G. Wermuth, Eds., Wiley-VCH, Weinheim, 2002.

As generally used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

Unless otherwise stated to the contrary, the teaching or disclosure of any of the compounds and compositions described herein should be understood to encompass the separate disclosure of each and every distinct stereoisomer. The compounds and compositions may be purified single stereoisomers, purified mixtures of only a few stereoisomers, or racemic mixtures of all stereoisomers. Methods of purifying stereoisomers include crystallization, chromatographic approaches, multi-phase extractions, or membrane filtration, among others. Such methods are known in the art. Likewise, the disclosure of any of the compounds or compositions herein should be understood to encompass all possible isotopic substitutions of the elements contained. The disclosed compounds or compositions may contain a mixture of all naturally occurring isotopes or may contain compounds where one or more element is substituted with a different isotope. For instance, one or my hydrogen atoms may be replaced with deuterium or tritium. Methods of isotopic labeling are known in the art. In some instances, the compounds may include all enantiomers thereof, derivatives thereof, or prodrugs thereof.

### EXAMPLES

### Example 1

### Synthesis of (2S,3R)-2-Amino-3-hydroxy-N-octylbutanamide

1.350 g (10 mmol) of 1-hydroxybenzotriazole hydrate (HOBt) was added at 0° C. to a solution of 2.190 g (10 mmol) of N-Boc-L-threonine in 60 mL of anhydrous tetrahydrofuran (THF). The pH of the solution was adjusted to 8-9 with 4-methylmorpholine. After the mixture was stirred for 5 min, 2.200 g (11 mmol) of 1-ethyl-3-(3-dimethyllaminopropyl)carbodiimide hydrochloride (EDC.HCl) was added. A solution of 10 mmol of 1-octyl amine in 5 mL of anhydrous THF was added to a solution of N-Boc-L-threonine, and the mixture was stirred at 0° C. for 2 h and at room temperature overnight. On evaporation the residue was dissolved in 60 mL of ethyl acetate. The solution was washed successively with saturated sodium bicarbonate, 5% potassium bisulfate, and saturated sodium chloride, and the organic phase was separated and dried over anhydrous magnesium sulfate for 2 h. After filtration and evaporation under reduced pressure (2S,3R)-tert-Butyl-3-hydroxy-1-oxo-1-(substituted)butan-2-ylcarbamate was obtained. At 0° C., to a solution of (2S,3R)-tert-Butyl-3-hydroxy-1-oxo-1-(substituted)butan-2-ylcarbamate in 70 mL of dichloromethane (DCM), 10.0 mL of trifluoroacetic acid was added. After the mixture was stirred for 4 h, the solvent was removed under vacuum pressure, and the residue was precipitated in 100 mL 1N NaOH solution. Filtration and dried over to give compound NZJU1.

The product was obtained as a colorless solid in a yield of 62.5%. ¹H NMR (300 MHz, CDCl₃) δ 7.41 (s, 1H), 4.34-4.16 (m, 1H), 3.37-3.09 (m, 3H), 1.57-1.43 (m, 2H), 1.38-1.22 (m, 10H), 1.19 (d, J=6.5 Hz, 3H), 0.87 (t, J=6.6 Hz, 3H); ¹³C NMR (75 MHz, CDCl₃) δ 173.7, 67.8, 59.6, 39.2, 31.8, 29.5, 29.3, 29.2, 27.0, 22.6, 19.0, 14.1; ESI/MS (m/e) 231.15 [M+H]⁺.

### Example 2

### Synthesis of (2S,3R)-2-Amino-3-hydroxy-N-dodecylbutanamide

1.350 g (10 mmol) of 1-hydroxybenzotriazole hydrate (HOBt) was added at 0° C. to a solution of 2.190 g (10 mmol) of N-Boc-L-threonine in 60 mL of anhydrous tetrahydrofuran (THF). The pH of the solution was adjusted to 8-9 with 4-methylmorpholine. After the mixture was stirred for 5 min, 2.200 g (11 mmol) of 1-ethyl-3-(3-dimethyllaminopropyl)carbodiimide hydrochloride (EDC.HCl) was added. A solution of 10 mmol of 1-dodecyl amine in 5 mL of anhydrous THF was added to a solution of N-Boc-L-threonine, and the mixture was stirred at 0° C. for 2 h and at room temperature overnight. On evaporation the residue was dissolved in 60 mL of ethyl acetate. The solution was washed successively with saturated sodium bicarbonate, 5% potassium bisulfate, and saturated sodium chloride, and the organic phase was separated and dried over anhydrous magnesium sulfate for 2 h. After filtration and evaporation under reduced pressure (2S,3R)-tert-Butyl-3-hydroxy-1-oxo-1-(substituted)butan-2-ylcarbamate was obtained. At 0° C., to a solution of (2S,3R)-tert-Butyl-3-hydroxy-1-oxo-1-(substituted)butan-2-ylcarbamate in 70 mL of dichloromethane (DCM), 10.0 mL of trifluoroacetic acid was added. After the mixture was stirred for 4 h, the solvent was removed under vacuum pressure, and the residue was precipitated in 100 mL 1N NaOH solution. Filtration and dried over to give compound NZJU2.

The product was obtained as a yellow solid in a yield of 62.3%. ¹H NMR (300 MHz, CDCl₃) δ 7.39 (s, 1H), 4.36-4.12 (m, 1H), 3.35-3.11 (m, 3H), 1.58-1.42 (m, 2H), 1.36-1.22 (m, 18H), 1.19 (d, J=6.5 Hz, 3H), 0.88 (t, J=6.6 Hz, 3H); ¹³C NMR (75 MHz, CDCl₃) δ 173.5, 67.8, 59.7, 39.3, 31.9, 29.7, 29.6, 29.5, 29.4, 29.3, 27.0, 22.7, 19.0, 14.1; ESI/MS (m/e) 287.67 [M+H]⁺.

### Example 3

### Synthesis of (2S,3R)-2-Amino-3-hydroxy-N-tetradecylbutanamide

1.350 g (10 mmol) of 1-hydroxybenzotriazole hydrate (HOBt) was added at 0° C. to a solution of 2.190 g (10 mmol) of N-Boc-L-threonine in 60 mL of anhydrous tetrahydrofuran (THF). The pH of the solution was adjusted to 8-9 with 4-methylmorpholine. After the mixture was stirred for 5 min, 2.200 g (11 mmol) of 1-ethyl-3-(3-dimethyllaminopropyl)carbodiimide hydrochloride (EDC.HCl) was added. A solution of 10 mmol of 1-tetradecyl amine in 5 mL of anhydrous THF was added to a solution of N-Boc-L-threonine, and the mixture was stirred at 0° C. for 2 h and at room temperature overnight. On evaporation the residue was dissolved in 60 mL of ethyl acetate. The solution was washed successively with saturated sodium bicarbonate, 5% potassium bisulfate, and saturated sodium chloride, and the organic phase was separated and dried over anhydrous magnesium sulfate for 2 h. After filtration and evaporation under reduced pressure (2S,3R)-tert-Butyl-3-hydroxy-1-oxo-1-(substituted)butan-2-ylcarbamate was obtained. At 0° C., to a solution of (2S,3R)-tert-Butyl-3-hydroxy-1-oxo-1-(substituted)butan-2-ylcarbamate in 70 mL of dichloromethane (DCM), 10.0 mL of trifluoroacetic acid was added. After the mixture was stirred for 4 h, the solvent was removed under vacuum pressure, and the residue was precipitated in 100 mL 1N NaOH solution. Filtration and dried over to give compound NZJU3.

The product was obtained as a pale yellow solid in a yield of 64.6%. ¹H NMR (500 MHz, CDCl₃) δ 7.42 (s, 1H), 4.35-4.22 (m, 1H), 3.33-3.25 (m, 2H), 3.24 (d, J=3.0 Hz, 1H), 1.58-1.47 (m, 2H), 1.38-1.24 (m, 22H), 1.21 (d, J=6.4 Hz, 3H), 0.90 (t, J=6.8 Hz, 3H); ¹³C NMR (75 MHz, CDCl₃) δ 173.6, 68.1, 59.4, 39.2, 31.9, 29.7, 29.6, 29.4, 29.3, 27.0, 22.7, 18.7, 14.1; ESI/MS (m/e) 315.58 [M+H]⁺.

### Example 4

### Synthesis of (2S,3R)-2-Amino-3-hydroxy-N-octadecylbutanamide

1.350 g (10 mmol) of 1-hydroxybenzotriazole hydrate (HOBt) was added at 0° C. to a solution of 2.190 g (10 mmol) of N-Boc-L-threonine in 60 mL of anhydrous tetrahydrofuran (THF). The pH of the solution was adjusted to 8-9 with 4-methylmorpholine. After the mixture was stirred for 5 min, 2.200 g (11 mmol) of 1-ethyl-3-(3-dimethyllaminopropyl)carbodiimide hydrochloride (EDC.HCl) was added. A solution of 10 mmol of 1-octadecyl amine in 5 mL of anhydrous THF was added to a solution of N-Boc-L-threonine, and the mixture was stirred at 0° C. for 2 h and at room temperature overnight. On evaporation the residue was dissolved in 60 mL of ethyl acetate. The solution was washed successively with saturated sodium bicarbonate, 5% potassium bisulfate, and saturated sodium chloride, and the organic phase was separated and dried over anhydrous magnesium sulfate for 2 h. After filtration and evaporation under reduced pressure (2S,3R)-tert-Butyl-3-hydroxy-1-oxo-1-(substituted)butan-2-ylcarbamate was obtained. At 0° C., to a solution of (2S,3R)-tert-Butyl-3-hydroxy-1-oxo-1-(substituted)butan-2-ylcarbamate in 70 mL of dichloromethane (DCM), 10.0 mL of trifluoroacetic acid was added. After the mixture was stirred for 4 h, the solvent was removed under vacuum pressure, and the residue was precipitated in 100 mL 1N NaOH solution. Filtration and dried over to give compound NZJU4.

The product was obtained as a pale colorless solid in a yield of 66.7%. ¹H NMR (300 MHz, CDCl₃) δ 7.40 (s, 1H), 4.35-4.20 (m, 1H), 3.35-3.17 (m, 3H), 1.59-1.43 (m, 2H), 1.34-1.22 (m, 30H), 1.19 (d, J=6.4 Hz, 3H), 0.88 (t, J=6.3 Hz, 3H); ¹³C NMR (75 MHz, CDCl₃) δ 173.6, 67.9, 59.5, 39.2, 31.9, 29.7, 29.6, 29.4, 29.3, 27.0, 22.7, 18.8, 14.1; ESI/MS (m/e) 371.67 [M+H]⁺.

### Example 5

### Condensation of NZJU1-NZJU4 with Benzoic Acid

At 0° C., to a solution of 1.0 mmol benzoic acid in anhydrous THF (20 mL), 0.135 g (1.0 mmol) of HOBt and 1.0 mmol of (2S,3R)-2-Amino-3-hydroxy-N-octylbutanamide, (2S,3R)-2-Amino-3-hydroxy-N-dodecylbutanamide, (2S,3R)-2-Amino-3-hydroxy-N-tetradecylbutanamide, or (2S,3R)-2-Amino-3-hydroxy-N-octadecylbutanamide were added. After 5 min, 0.220 g (1.1 mmol) of EDC.HCl was added, and the pH of the solution was adjusted to 8-9 with 4-methylmorpholine. The mixture was stirred at 0° C. for 2 h and at room temperature overnight. On evaporation the residue was dissolved in 80 mL of ethyl acetate. The solution was washed successively with saturated sodium bicarbonate, 5% potassium bisulfate, and saturated sodium chloride, and the organic phase was separated and dried over anhydrous magnesium sulfate for 2 h. After filtration and evaporation under reduced pressure crude product was obtained and recrystallized using ethyl acetate to obtain compounds NZJU1a, NZJU2a, NZJU3a, and NZJU4a.

N-((2S,3R)-3-hydroxy-1-oxo-1-(octylamino)butan-2-yl)-benzamide. (NZJU1a) was obtained in a yield of 0.259 g (77.5%) as colorless powder. ¹H NMR (300 MHz, CDCl₃) δ 7.84 (s, 1H), 7.81 (d, J=1.5 Hz, 1H), 7.57-7.52 (m, 1H), 7.51-7.41 (m, 2H), 7.19 (d, J=7.5 Hz, 1H), 6.89 (s, 1H), 4.58-4.40 (m, 2H), 3.34-3.16 (m, 2H), 1.56-1.44 (m, 2H), 1.35-1.13 (m, 13H), 0.86 (t, J=6.7 Hz, 3H); ¹³C NMR (75 MHz, CDCl₃) δ 171.2, 168.5, 133.3, 132.2, 128.8, 127.1, 66.4, 56.8, 39.6, 31.8, 29.4, 29.2, 26.9, 22.6, 18.2, 14.1; ESI/MS (m/e) 335 [M+H]⁺; Anal. Calcd. For C₁₉H₃₀N₂O₃: C, 68.23; H, 9.04; N, 8.38%. Found: C, 68.19; H, 9.07; N, 8.42%.

N-((2S,3R)-3-hydroxy-1-oxo-1-(dodecylamino)butan-2-yl)-benzamide (NZJU2a) was obtained in a yield of 0.315 g (80.7%) as colorless powder. ¹H NMR (300 MHz, CDCl₃) δ 7.87-7.74 (m, 2H), 7.63-7.48 (m, 1H), 7.49-7.29 (m, 3H), 7.05 (s, 1H), 4.62-4.38 (m, 2H), 3.33-3.15 (m, 2H), 1.54-1.39 (m, 2H), 1.36-1.09 (m, 21H), 0.88 (t, J=6.5 Hz, 3H); ¹³C NMR (75 MHz, CDCl₃) δ 170.9, 168.1, 133.7, 131.9, 128.6, 127.2, 67.1, 57.9, 39.7, 31.9, 29.6, 29.5, 29.4, 29.3, 26.9, 22.7, 18.3, 14.1; ESI/MS (m/e) 391.25 [M+H]⁺; Anal. Calcd. For C₂₃H₃₈N₂O₃: C, 70.73; H, 9.81; N, 7.17%. Found: C, 70.82; H, 9.73; N, 7.16%.

N-((2S,3R)-3-hydroxy-1-oxo-1-(tetradecylamino)butan-2-yl)-benzamide (NZJU3a) was obtained in a yield of 0.330 g (78.9%) as colorless powder. ¹H NMR (300 MHz, CDCl₃) δ 7.88-7.75 (m, 2H), 7.59-7.50 (m, 1H), 7.49-7.27 (m, 3H), 7.00 (s, 1H), 4.58-4.41 (m, 2H), 3.32-3.16 (m, 2H), 1.57-1.40 (m, 2H), 1.37-1.10 (m, 25H), 0.88 (t, J=6.6 Hz, 3H); ¹³C NMR (75 MHz, CDCl₃) δ 171.3, 168.4, 133.4, 132.2, 128.7, 127.2, 66.5, 57.0, 39.6, 31.9, 29.7, 29.6, 29.5, 29.4, 29.3, 26.9, 22.7, 18.2, 14.1; ESI/MS (m/e) 441.67 [M+Na]⁺; Anal. Calcd. For C₂₅H₄₂N₂O₃: C, 71.73; H, 10.11; N, 6.69%. Found: C, 71.70; H, 9.90; N, 6.65%.

N-((2S,3R)-3-hydroxy-1-oxo-1-(octadecylamino)butan-2-yl)-benzamide (NZJU4a) was obtained in a yield of 0.401 g (84.5%) as colorless powder. ¹H NMR (300 MHz, CDCl₃) δ 7.90-7.81 (m, 2H), 7.61-7.51 (m, 1H), 7.50-7.37 (m, 3H), 7.23 (s, 1H), 4.61-4.53 (m, 1H), 4.48 (d, J=6.1 Hz, 1H), 3.34-3.16 (m, 2H), 1.59-1.43 (m, 2H), 1.36-1.15 (m, 33H), 0.88 (t, J=6.5 Hz, 3H); ¹³C NMR (75 MHz, CDCl₃) δ 171.5, 168.6, 133.1, 132.3, 128.7, 127.2, 66.5, 57.1, 39.7, 31.9, 29.7, 29.6, 29.5, 29.4, 29.3, 29.2, 26.9, 22.7, 18.3, 14.1; ESI/MS (m/e) 497.50 [M+Na]⁺; Anal. Calcd. For C₂₉H₅₀N₂O₃: C, 73.37; H, 10.62; N, 5.90%. Found: C, 73.34; H, 10.49; N, 5.96%.

### Example 6

### Condensation of NZJU1-NZJU4 with Furan-2-Carboxylic Acid

At 0° C., to a solution of 1.0 mmol of furan-2-carboxylic acid in anhydrous THF (20 mL), 0.135 g (1.0 mmol) of HOBt and 1.0 mmol of (2S,3R)-2-Amino-3-hydroxy-N-octylbutanamide, (2S,3R)-2-Amino-3-hydroxy-N-dodecylbutanamide, (2S,3R)-2-Amino-3-hydroxy-N-tetradecylbutanamide, or (2S,3R)-2-Amino-3-hydroxy-N-octadecylbutanamide were added. After 5 min, 0.220 g (1.1 mmol) of EDC.HCl was added, and the pH of the solution was adjusted to 8-9 with 4-methylmorpholine. The mixture was stirred at 0° C. for 2 h and at room temperature overnight. On evaporation the residue was dissolved in 80 mL of ethyl acetate. The solution was washed successively with saturated sodium bicarbonate, 5% potassium bisulfate, and saturated sodium chloride, and the organic phase was separated and dried over anhydrous magnesium sulfate for 2 h. After filtration and evaporation under reduced pressure crude product was obtained and recrystallized using ethyl acetate to obtain compounds NZJU1b, NZJU2b, NZJU3b, and NZJU4b.

N-((2S,3R)-3-hydroxy-1-oxo-1-(octylamino)butan-2-yl)-Furan-2-carboxamide (NZJU1b) was obtained in a yield of 0.262 g (80.9%) as colorless powder. ¹H NMR (300 MHz, CDCl₃) δ 7.49 (s, 1H), 7.33 (d, J=7.5 Hz, 1H), 7.14 (d, J=3.0 Hz, 1H), 6.84 (s, 1H), 6.52 (dd, J=3.5, 1.7 Hz, 1H), 4.53-4.39 (m, 2H), 3.34-3.09 (m, 2H), 1.56-1.42 (m, 2H), 1.37-1.11 (m, 13H), 0.86 (t, J=6.7 Hz, 3H); ¹³C NMR (75 MHz, CDCl₃) δ 170.7, 159.4, 147.1, 144.7, 115.1, 112.2, 66.5, 56.5, 39.6, 31.8, 29.4, 29.2, 26.9, 22.6, 18.1, 14.1; ESI/MS (m/e) 325.00 [M+H]⁺; Anal. Calcd. For C₁₇H₂₈N₂O₄: C, 62.94; H, 8.70; N, 8.64%. Found: C, 62.89; H, 8.84; N, 8.69%.

N-((2S,3R)-3-hydroxy-1-oxo-1-(dodecylamino)butan-2-yl)-Furan-2-carboxamide (NZJU2b) was obtained in a yield of 0.302 g (79.4%) as colorless powder. ¹H NMR (300 MHz, CDCl₃) δ 7.48 (s, 1H), 7.36 (d, J=7.5 Hz, 1H), 7.13 (d, J=3.0 Hz, 1H), 6.88 (s, 1H), 6.52 (dd, J=3.5, 1.7 Hz, 1H), 4.57-4.36 (m, 2H), 3.36-3.10 (m, 2H), 1.56-1.39 (m, 2H), 1.36-1.11 (m, 21H), 0.87 (t, J=6.7 Hz, 3H); ¹³C NMR (75 MHz, CDCl₃) δ 170.9, 159.2, 147.0, 144.7, 115.1, 112.2, 66.4, 56.3, 39.6, 31.9, 29.6, 29.5, 29.4, 29.2, 26.9, 22.7, 18.1, 14.1; ESI/MS (m/e) 403.50 [M+Na]⁺; Anal. Calcd. For C₂₁H₃₆N₂O₄: C, 66.28; H, 9.54; N, 7.36%. Found: C, 66.17; H, 9.64; N, 7.43%.

N-((2S,3R)-3-hydroxy-1-oxo-1-(tetradecylamino)butan-2-yl)-Furan-2-carboxamide (NZJU3b) was obtained in a yield of 0.343 g (84.0%) as colorless powder. ¹H NMR (300 MHz, CDCl₃) δ 7.49 (s, 1H), 7.34 (d, J=7.0 Hz, 1H), 7.15 (d, J=3.3 Hz, 1H), 6.84 (s, 1H), 6.57-6.42 (m, 1H), 4.53-4.38 (m, 2H), 3.37-3.07 (m, 2H), 1.56-1.38 (m, 2H), 1.37-1.08 (m, 25H), 0.88 (t, J=6.6 Hz, 3H); ¹³C NMR (75 MHz, CDCl₃) δ 171.0, 159.3, 147.1, 144.7, 115.1, 112.3, 66.3, 56.2, 39.6, 31.9, 29.7, 29.6, 29.5, 29.4, 29.3, 26.9, 22.7, 18.2, 14.1; ESI/MS (m/e) 431.50 [M+Na]⁺; Anal. Calcd. For C₂₃H₄₀N₂O₄: C, 67.61; H, 9.87; N, 6.86%. Found: C, 67.68; H, 9.77; N, 6.92%.

N-((2S,3R)-3-hydroxy-1-oxo-1-(octadecylamino)butan-2-yl)-Furan-2-carboxamide (NZJU4b) was obtained in a yield of 0.398 g (85.7%) as colorless powder. ¹H NMR (300 MHz, CDCl₃) δ 7.50 (s, 1H), 7.31 (d, J=7.9 Hz, 1H), 7.15 (d, J=3.3 Hz, 1H), 6.79 (s, 1H), 6.53 (dd, J=3.3, 1.7 Hz, 1H), 4.55-4.45 (m, 1H), 4.42 (d, J=8.3 Hz, 1H), 3.36-3.14 (m, 2H), 1.58-1.44 (m, 2H), 1.36-1.11 (m, 33H), 0.88 (t, J=6.6 Hz, 4H); ¹³C NMR (75 MHz, CDCl₃) δ 171.2, 159.3, 146.5, 144.8, 115.1, 112.3, 66.2, 56.0, 39.6, 31.9, 29.7, 29.6, 29.5, 29.4, 29.2, 26.9, 22.7, 18.2, 14.1; ESI/MS (m/e) 487.58 [M+Na]⁺; Anal. Calcd. For C₂₇H₄₈N₂O₄: C, 69.79; H, 10.41; N, 6.03%. Found: C, 69.81; H, 10.43; N, 6.12%.

### Example 7

### Condensation of NZJU1-NZJU4 with (2E,4E)-hexa-2,4-dienoic Acid

At 0° C., to a solution of 1.0 mmol of (2E,4E)-hexa-2,4-dienoic acid in anhydrous THF (20 mL), 0.135 g (1.0 mmol) of HOBt and 1.0 mmol of (2S,3R)-2-Amino-3-hydroxy-N-octylbutanamide, (2S,3R)-2-Amino-3-hydroxy-N-dodecylbutanamide, (2S,3R)-2-Amino-3-hydroxy-N-tetradecylbutanamide, or (2S,3R)-2-Amino-3-hydroxy-N-octadecylbutanamide were added. After 5 min, 0.220 g (1.1 mmol) of EDC.HCl was added, and the pH of the solution was adjusted to 8-9 with 4-methylmorpholine. The mixture was stirred at 0° C. for 2 h and at room temperature overnight. On evaporation the residue was dissolved in 80 mL of ethyl acetate. The solution was washed successively with saturated sodium bicarbonate, 5% potassium bisulfate, and saturated sodium chloride, and the organic phase was separated and dried over anhydrous magnesium sulfate for 2 h. After filtration and evaporation under reduced pressure crude product was obtained and recrystallized using ethyl acetate to obtain compounds NZJU1c, NZJU2c, NZJU3c, and NZJU4c.

(2E,4E)-N-((2S,3R)-3-hydroxy-1-oxo-1-(octylamino)butan-2-yl)-2,4-hexadienoic acid amide (NZJU1c) was obtained in a yield of 0.270 g (83.3%) as colorless powder. ¹H NMR (300 MHz, CDCl₃) δ 7.24-7.15 (m, 1H), 6.87 (s, 1H), 6.52 (d, J=6.2 Hz, 1H), 6.26-6.04 (m, 2H), 5.83 (d, J=15.1 Hz, 1H), 4.51-4.38 (m, 1H), 4.35 (d, J=7.8 Hz, 1H), 3.29-3.13 (m, 2H), 1.86 (d, J=5.3 Hz, 3H), 1.55-1.41 (m, 2H), 1.36-1.20 (m, 10H), 1.16 (d, J=6.5 Hz, 3H), 0.87 (t, J=6.6 Hz, 3H); ¹³C NMR (75 MHz, CDCl₃) δ 171.5, 167.6, 142.4, 138.9, 129.6, 120.6, 66.5, 56.8, 39.5, 31.8, 29.4, 29.2, 26.9, 22.6, 18.6, 18.1, 14.1; ESI/MS (m/e) 325 [M+H]⁺; Anal. Calcd. For C₁₈H₃₂N₂O₃: C, 66.63; H, 9.94; N, 8.63%. Found: C, 66.57; H, 10.03; N, 8.74%.

(2E,4E)-N-((2S,3R)-3-hydroxy-1-oxo-1-(dodecylamino)butan-2-yl)-2,4-hexadienoic acid amide (NZJU2c) was obtained in a yield of 0.310 g (81.6%) as colorless powder. ¹H NMR (300 MHz, CDCl₃) δ 7.24-7.15 (m, 1H), 6.95 (s, 1H), 6.64 (s, 1H), 6.28-6.05 (m, 2H), 5.85 (d, J=15.1 Hz, 1H), 4.48-4.28 (m, 2H), 3.31-3.11 (m, 2H), 1.86 (d, J=5.3 Hz, 3H), 1.57-1.42 (m, 2H), 1.40-1.20 (m, 18H), 1.17 (d, J=6.4 Hz, 3H), 0.88 (t, J=6.7 Hz, 3H); ¹³C NMR (75 MHz, CDCl₃) δ 171.6, 167.5, 142.6, 139.1, 129.6, 120.3, 66.3, 56.4, 39.5, 32.0, 29.7, 29.6, 29.4, 29.3, 26.9, 22.7, 18.7, 18.1, 14.2. ESI/MS (m/e) 403.42 [M+Na]⁺; Anal. Calcd. For C₂₂H₄₀N₂O₃: C, 69.43; H, 10.59; N, 7.36%. Found: C, 69.33; H, 10.64; N, 7.26%.

(2E,4E)-N-((2S,3R)-3-hydroxy-1-oxo-1-(tetradecylamino)butan-2-yl)-2,4-hexadienoic acid amide (NZJU3c) was obtained in a yield of 0.335 g (82.1%) as colorless powder. ¹H NMR (300 MHz, CDCl₃) δ 7.24-7.13 (m, 1H), 6.87 (s, 1H), 6.54 (d, J=8.0 Hz, 1H), 6.25-6.02 (m, 2H), 5.84 (d, J=15.0 Hz, 1H), 4.49-4.26 (m, 2H), 3.30-3.09 (m, 2H), 1.86 (d, J=5.3 Hz, 3H), 1.56-1.40 (m, 2H), 1.39-1.19 (m, 22H), 1.16 (d, J=6.5 Hz, 3H), 0.88 (t, J=6.6 Hz, 3H); ¹³C NMR (75 MHz, CDCl₃) δ 171.5, 167.5, 142.5, 139.0, 129.6, 120.4, 66.3, 56.4, 39.5, 31.9, 29.7, 29.6, 29.6, 29.4, 29.3, 26.9, 22.7, 18.7, 18.1, 14.1; ESI/MS (m/e) 431.42 [M+Na]⁺; Anal. Calcd. For C₂₄H₄₄N₂O₃: C, 70.54; H, 10.85; N, 6.86%. Found: C, 70.45; H, 10.78; N, 6.81%.

(2E,4E)-N-((2S,3R)-3-hydroxy-1-oxo-1-(octadecylamino)butan-2-yl)-2,4-hexadienoic acid amide (NZJU4c) was obtained in a yield of 0.392 g (84.5%) as colorless powder. ¹H NMR (300 MHz, CDCl₃) δ 7.26-7.15 (m, 1H), 6.89 (s, 1H), 6.56 (s, 1H), 6.31-6.05 (m, 2H), 5.83 (d, J=15.1 Hz, 1H), 4.51-4.30 (m, 2H), 3.28-3.13 (m, 2H), 1.86 (d, J=5.3 Hz, 7H), 1.55-1.42 (m, 2H), 1.38-1.20 (m, 30H), 1.17 (d, J=6.5 Hz, 3H), 0.88 (t, J=6.7 Hz, 3H); ¹³C NMR (75 MHz, CDCl₃) δ 171.6, 167.7, 142.8, 139.3, 129.5, 120.1, 66.3, 56.5, 39.6, 32.0, 29.7, 29.6, 29.6, 29.4, 29.3, 26.9, 22.7, 18.7, 18.2, 14.2; ESI/MS (m/e) 487.50 [M+Na]⁺; Anal. Calcd. For C₂₈H₅₂N₂O₃: C, 72.37; H, 11.28; N, 6.03%. Found: C, 72.29; H, 11.21; N, 5.98%.

### Example 8

### Condensation of NZJU1-NZJU4 with Cinnamic Acid

At 0° C., to a solution of 1.0 mmol of cinnamic acid in anhydrous THF (20 mL), 0.135 g (1.0 mmol) of HOBt and 1.0 mmol (2S,3R)-2-Amino-3-hydroxy-N-octylbutanamide, (2S,3R)-2-Amino-3-hydroxy-N-dodecylbutanamide, (2S,3R)-2-Amino-3-hydroxy-N-tetradecylbutanamide, or (2S,3R)-2-Amino-3-hydroxy-N-octadecylbutanamide were added. After 5 min, 0.220 g (1.1 mmol) of EDC.HCl was added, and the pH of the solution was adjusted to 8-9 with 4-methylmorpholine. The mixture was stirred at 0° C. for 2 h and at room temperature overnight. On evaporation the residue was dissolved in 80 mL of ethyl acetate. The solution was washed successively with saturated sodium bicarbonate, 5% potassium bisulfate, and saturated sodium chloride, and the organic phase was separated and dried over anhydrous magnesium sulfate for 2 h. After filtration and evaporation under reduced pressure crude product was obtained and recrystallized using ethyl acetate to obtain compounds NZJU1d, NZJU2d, NZJU3d, and NZJU4d.

(E)-N-((2S,3R)-3-hydroxy-1-oxo-1-(octylamino)butan-2-yl)-3-phenyl acrylic acid amide (NZJU1d) was obtained in a yield of 0.291 g (80.8%) as colorless powder. ¹H NMR (300 MHz, CDCl₃) δ 7.66 (d, J=15.8 Hz, 1H), 7.51 (d, J=3.7 Hz, 2H), 7.42-7.30 (m, 3H), 6.95 (s, 1H), 6.89-6.81 (m, 1H), 6.53 (d, J=15.6 Hz, 1H), 4.53-4.33 (m, 2H), 3.33-3.13 (m, 2H), 1.57-1.39 (m, 2H), 1.37-1.06 (m, 13H), 0.85 (t, J=6.6 Hz, 3H); ¹³C NMR (75 MHz, CDCl₃) δ 170.9, 166.9, 141.9, 134.5, 129.9, 128.8, 127.9, 120.1, 67.0, 57.3, 39.7, 31.8, 29.4, 29.2, 27.0, 22.6, 18.2, 14.1; ESI/MS (m/e) 361 [M+H]⁺; Anal. Calcd. For C₂₁H₃₂N₂O₃: C, 69.97; H, 8.95; N, 7.77%. Found: C, 69.83; H, 8.87; N, 7.69%.

(E)-N-((2S,3R)-3-hydroxy-1-oxo-1-(dodecylamino)butan-2-yl)-3-phenyl acrylic acid amide (NZJU2d) was obtained in a yield of 0.327 g (78.6%) as colorless powder. ¹H NMR (300 MHz, CDCl₃) δ 7.65 (d, J=15.6 Hz, 1H), 7.50 (d, J=5.4 Hz, 2H), 7.43-7.30 (m, 3H), 6.98 (s, 1H), 6.88 (d, J=4.2 Hz, 1H), 6.54 (d, J=15.5 Hz, 1H), 4.54-4.34 (m, 2H), 3.34-3.12 (m, 2H), 1.56-1.40 (m, 2H), 1.22 (s, 21H), 0.87 (t, J=6.6 Hz, 3H); ¹³C NMR (75 MHz, CDCl₃) δ 171.5, 167.2, 142.3, 134.3, 130.1, 128.9, 128.0, 119.6, 66.4, 56.5, 39.6, 31.9, 29.7, 29.6, 29.6, 29.4, 29.3, 26.9, 22.7, 17.9, 14.2; ESI/MS (m/e) 417 [M+H]⁺; Anal. Calcd. For C₂₅H₄₀N₂O₃: C, 72.08; H, 9.68; N, 6.72%. Found: C, 72.12; H, 9.71; N, 6.70%.

(E)-N-((2S,3R)-3-hydroxy-1-oxo-1-(tetradecylamino)butan-2-yl)-3-phenyl acrylic acid amide (NZJU3d) was obtained in a yield of 0.352 g (79.3%) as colorless powder. ¹H NMR (300 MHz, CDCl₃) δ 7.67 (d, J=15.6 Hz, 1H), 7.52 (d, J=3.7 Hz, 2H), 7.43-7.31 (m, 3H), 6.92 (s, 1H), 6.78 (d, J=5.1 Hz, 1H), 6.51 (d, J=15.6 Hz, 1H), 4.53-4.33 (m, 2H), 3.33-3.14 (m, 2H), 1.57-1.41 (m, 2H), 1.40-1.07 (m, 25H), 0.88 (t, J=6.6 Hz, 3H); ¹³C NMR (75 MHz, CDCl₃) δ 171.2, 167.0, 142.1, 134.5, 130.0, 128.9, 128.0, 119.8, 66.6, 56.8, 39.6, 31.9, 29.7, 29.6, 29.4, 29.3, 26.9, 22.7, 18.1, 14.1; ESI/MS (m/e) 445 [M+H]⁺; Anal. Calcd. For C₂₇H₄₄N₂O₃: C, 72.93; H, 9.97; N, 6.30%. Found: C, 72.97; H, 9.91; N, 6.27%.

(E)-N-((2S,3R)-3-hydroxy-1-oxo-1-(octadecylamino)butan-2-yl)-3-phenyl acrylic acid amide (NZJU4d) was obtained in a yield of 0.389 g (77.8%) as colorless powder. ¹H NMR (300 MHz, CDCl₃) δ 7.66 (d, J=15.6 Hz, 1H), 7.52 (d, J=3.5 Hz, 2H), 7.45-7.33 (m, 3H), 6.89 (s, 1H), 6.74 (d, J=6.9 Hz, 1H), 6.51 (d, J=15.6 Hz, 1H), 4.60-4.36 (m, 2H), 3.37-3.14 (m, 2H), 1.61-1.44 (m, 2H), 1.40-1.07 (m, 33H), 0.88 (t, J=6.4 Hz, 3H); ¹³C NMR (75 MHz, CDCl₃) δ 171.3, 167.0, 142.3, 134.5, 130.1, 128.9, 128.0, 119.7, 66.5, 56.7, 39.6, 31.9, 29.7, 29.6, 29.5, 29.4, 29.3, 26.9, 22.7, 18.1, 14.1; ESI/MS (m/e) 501 [M+H]⁺; Anal. Calcd. For C₃₁H₅₂N₂O₃: C, 74.35; H, 10.47; N, 5.59%. Found: C, 74.37; H, 10.49; N, 5.52%.

### Example 9

### Condensation of NZJU2-NZJU4 with 2-(1H-indol-3-yl)acetic Acid

At 0° C., to a solution of 1.0 mmol of 2-(1H-indol-3-yl)acetic acid in anhydrous THF (20 mL), 0.135 g (1.0 mmol) of HOBt and 1.0 mmol of (2S,3R)-2-Amino-3-hydroxy-N-dodecylbutanamide, (2S,3R)-2-Amino-3-hydroxy-N-tetradecylbutanamide, or (2S,3R)-2-Amino-3-hydroxy-N-octadecylbutanamide were added. After 5 min, 0.220 g (1.1 mmol) of EDC.HCl was added, and the pH of the solution was adjusted to 8-9 with 4-methylmorpholine. The mixture was stirred at 0° C. for 2 h and at room temperature overnight. On evaporation the residue was dissolved in 80 mL of ethyl acetate. The solution was washed successively with saturated sodium bicarbonate, 5% potassium bisulfate, and saturated sodium chloride, and the organic phase was separated and dried over anhydrous magnesium sulfate for 2 h. After filtration and evaporation under reduced pressure crude product was obtained and recrystallized using ethyl acetate to obtain compounds NZJU2e, NZJU3e, and NZJU4e.

N-((2S,3R)-3-hydroxy-1-oxo-1-(dodecylamino)butan-2-yl)-indole-3-acetic acid amide (NZJU2e) was obtained in a yield of 0.318 g (71.8%) as purple powder. ¹H NMR (300 MHz, CDCl₃) δ 8.53 (s, 1H), 7.53 (d, J=7.8 Hz, 1H), 7.34 (d, J=8.1 Hz, 1H), 7.24-7.16 (m, 1H), 7.15-7.03 (m, 2H), 6.81-6.67 (m, 2H), 4.39-4.19 (m, 2H), 3.78 (s, 2H), 3.20-3.00 (m, 2H), 1.49-1.33 (m, 2H), 1.32-1.08 (m, 18H), 0.95 (d, J=0.9 Hz, 3H), 0.88 (t, J=6.6 Hz, 3H); ¹³C NMR (75 MHz, CDCl₃) δ 173.2, 171.0, 136.5, 126.9, 123.9, 122.4, 119.8, 118.3, 111.7, 108.1, 66.5, 56.9, 39.6, 33.4, 31.9, 29.7, 29.6, 29.4, 29.3, 26.9, 22.7, 17.9, 14.2; ESI/MS (m/e) 442 [M−H]⁻; Anal. Calcd. For C₂₆H₄₁N₃O₃: C, 70.39; H, 9.32; N, 9.47%. Found: C, 70.27; H, 9.46; N, 9.39%.

N-((2S,3R)-3-hydroxy-1-oxo-1-(tetradecylamino)butan-2-yl)-indole-3-acetic acid amide (NZJU3e) was obtained in a yield of 0.305 g (64.8%) as purple powder. ¹H NMR (300 MHz, CDCl₃) δ 8.46 (s, 1H), 7.53 (d, J=7.6 Hz, 1H), 7.35 (d, J=7.7 Hz, 1H), 7.24-7.16 (m, 1H), 7.16-7.04 (m, 2H), 6.84-6.58 (m, 2H), 4.46-4.15 (m, 2H), 3.78 (s, 2H), 3.33-2.88 (m, 2H), 1.53-1.36 (m, 2H), 1.36-1.09 (m, 22H), 0.94 (d, J=1.9 Hz, 3H), 0.88 (t, J=5.9 Hz, 3H); ¹³C NMR (75 MHz, CDCl₃) δ 173.3, 170.8, 136.5, 126.9, 124.0, 122.4, 119.8, 118.3, 111.7, 108.0, 66.6, 57.2, 39.6, 33.5, 32.0, 29.7, 29.6, 29.4, 29.3, 26.9, 22.7, 18.1, 14.2; ESI/MS (m/e) 470 [M−H]⁻; Anal. Calcd. For C₂₈H₄₅N₃O₃: C, 71.30; H, 9.62; N, 8.91%. Found: C, 71.29; H, 9.71; N, 8.87%.

N-((2S,3R)-3-hydroxy-1-oxo-1-(octadecylamino)butan-2-yl)-indole-3-acetic acid amide (NZJU4e) was obtained in a yield of 0.406 g (77.0%) as colorless powder. ¹H NMR (300 MHz, CDCl₃) δ 8.39 (s, 1H), 7.53 (d, J=7.6 Hz, 1H), 7.36 (d, J=7.8 Hz, 1H), 7.25-7.17 (m, 1H), 7.17-7.09 (m, 2H), 6.73-6.59 (m, 2H), 4.35-4.20 (m, 2H), 3.79 (s, 2H), 3.19-3.04 (m, 2H), 1.49-1.34 (m, 2H), 1.33-1.12 (m, 30H), 0.94 (d, J=5.9 Hz, 3H), 0.88 (t, J=6.2 Hz, 3H); ¹³C NMR (75 MHz, CDCl₃) δ 173.0, 170.9, 136.5, 126.8, 123.8, 122.5, 119.9, 118.3, 111.6, 108.2, 66.3, 56.8, 39.5, 33.4, 32.0, 29.7, 29.6, 29.4, 29.3, 26.9, 22.7, 18.0, 14.2; ESI/MS (m/e) 550 [M+Na]⁺; Anal. Calcd. For C₃₂H₅₃N₃O₃: C, 72.82; H, 10.12; N, 7.96%. Found: C, 72.72; H, 10.07; N, 7.87%.

### Example 10

### Condensation of NZJU1-NZJU4 with Isoquinoline-1-carboxylic Acid

At 0° C., to a solution of 1.0 mmol of isoquinoline-1-carboxylic acid in anhydrous THF (20 mL), 0.135 g (1.0 mmol) of HOBt and 1.0 mmol of (2S,3R)-2-Amino-3-hydroxy-N-octylbutanamide, (2S,3R)-2-Amino-3-hydroxy-N-dodecylbutanamide, (2S,3R)-2-Amino-3-hydroxy-N-tetradecylbutanamide, or (2S,3R)-2-Amino-3-hydroxy-N-octadecylbutanamide were added. After 5 min, 0.220 g (1.1 mmol) of EDC.HCl was added, and the pH of the solution was adjusted to 8-9 with 4-methylmorpholine. The mixture was stirred at 0° C. for 2 h and at room temperature overnight. On evaporation the residue was dissolved in 80 mL of ethyl acetate. The solution was washed successively with saturated sodium bicarbonate, 5% potassium bisulfate, and saturated sodium chloride, and the organic phase was separated and dried over anhydrous magnesium sulfate for 2 h. After filtration and evaporation under reduced pressure crude product was obtained and recrystallized using ethyl acetate to obtain compounds NZJU1f, NZJU2f, NZJU3f, and NZJU4f.

N-((2S,3R)-3-hydroxy-1-oxo-1-(octylamino)butan-2-yl)-isoquinoline-1-carboxamide (NZJU1f) was obtained in a yield of 0.258 g (67.0%) as colorless powder. ¹H NMR (300 MHz, CDCl₃) δ 9.51 (d, J=8.1 Hz, 1H), 9.08 (d, J=8.0 Hz, 1H), 8.52 (d, J=5.5 Hz, 1H), 7.92-7.79 (m, 2H), 7.79-7.61 (m, 2H), 6.89 (s, 1H), 4.66-4.46 (m, 2H), 3.32-3.11 (m, 2H), 1.56-1.42 (m, 2H), 1.34-1.04 (m, 13H), 0.82 (t, J=6.7 Hz, 3H); ¹³C NMR (75 MHz, CDCl₃) δ 171.4, 167.5, 147.2, 140.6, 137.5, 130.6, 128.8, 127.3, 127.0, 124.8, 66.6, 56.9, 39.6, 31.7, 29.4, 29.2, 26.9, 22.6, 18.6, 14.1; ESI/MS (m/e) 386.20 [M+H]⁺; Anal. Calcd. For C₂₂H₃₁N₃O₃: C, 68.54; H, 8.11; N, 10.90%. Found: C, 68.47; H, 8.24; N, 10.86%.

N-((2S,3R)-3-hydroxy-1-oxo-1-(dodecylamino)butan-2-yl)-isoquinoline-1-carboxamide (NZJU2f) was obtained in a yield of 0.310 g (70.3%) as colorless powder. ¹H NMR (300 MHz, CDCl₃) δ 9.51 (d, J=7.6 Hz, 1H), 9.08 (d, J=7.8 Hz, 1H), 8.52 (d, J=5.2 Hz, 1H), 7.95-7.80 (m, 2H), 7.80-7.62 (m, 2H), 6.89 (s, 1H), 4.67-4.45 (m, 2H), 3.35-3.09 (m, 2H), 1.57-1.41 (m, 2H), 1.37-0.98 (m, 21H), 0.87 (t, J=6.4 Hz, 3H); ¹³C NMR (75 MHz, CDCl₃) δ 171.4, 167.3, 147.3, 140.6, 137.4, 130.5, 128.8, 127.3, 127.0, 124.7, 66.6, 56.9, 39.6, 31.9, 29.6, 29.5, 29.4, 29.3, 26.9, 22.7, 18.6, 14.1; ESI/MS (m/e) 442 [M+H]⁺; Anal. Calcd. For C₂₆H₃₉N₃O₃: C, 70.71; H, 8.90; N, 9.52%. Found: C, 70.68; H, 8.86; N, 9.49%.

N-((2S,3R)-3-hydroxy-1-oxo-1-(tetradecylamino)butan-2-yl)-isoquinoline-1-carboxamide (NZJU3f) was obtained in a yield of 0.317 g (67.6%) as colorless powder. ¹H NMR (300 MHz, CDCl₃) δ 9.51 (d, J=8.3 Hz, 1H), 9.07 (d, J=8.1 Hz, 1H), 8.52 (d, J=5.5 Hz, 1H), 7.94-7.79 (m, 2H), 7.78-7.60 (m, 2H), 6.89 (s, 1H), 4.68-4.46 (m, 2H), 3.35-3.11 (m, 2H), 1.58-1.39 (m, 2H), 1.39-1.00 (m, 25H), 0.88 (t, J=6.4 Hz, 3H); ¹³C NMR (75 MHz, CDCl₃) δ 171.2, 167.2, 147.4, 140.6, 137.4, 130.5, 128.7, 127.3, 127.0, 124.7, 66.7, 57.2, 39.6, 31.9, 29.7, 29.6, 29.4, 29.3, 28.3, 26.9, 22.7, 18.6, 14.1; ESI/MS (m/e) 470 [M+H]⁺; Anal. Calcd. For C₂₈H₄₃N₃O₃: C, 71.61; H, 9.23; N, 8.95%. Found: C, 71.58; H, 9.19; N, 8.98%.

N-((2S,3R)-3-hydroxy-1-oxo-1-(octadecylamino)butan-2-yl)-isoquinoline-1-carboxamide (NZJU4f) was obtained in a yield of 0.409 g (77.3%) as colorless powder. ¹H NMR (300 MHz, CDCl₃) δ 9.46 (d, J=8.3 Hz, 1H), 9.08 (d, J=7.4 Hz, 1H), 8.52 (d, J=5.2 Hz, 1H), 7.94-7.83 (m, 2H), 7.81-7.62 (m, 2H), 6.95 (s, 1H), 4.68-4.47 (m, 2H), 3.37-3.15 (m, 2H), 1.54-1.40 (m, 2H), 1.38-1.00 (m, 33H), 0.88 (t, J=6.2 Hz, 3H); ¹³C NMR (75 MHz, CDCl₃) δ 171.5, 167.4, 147.4, 140.5, 137.4, 130.6, 128.8, 127.3, 127.0, 124.7, 66.7, 57.0, 39.6, 31.9, 29.7, 29.6, 29.4, 29.3, 26.9, 22.7, 18.6, 14.1; ESI/MS (m/e) 526 [M+H]⁺; Anal. Calcd. For C₃₂H₅₁N₃O₃: C, 73.10; H, 9.78; N, 7.99%. Found: C, 73.07; H, 9.86; N, 8.02%.

### Example 11

### Condensation of NZJU1-NZJU4 with Thiophene-2-carboxylic Acid

At 0° C., to a solution of 1.0 mmol of thiophene-2-carboxylic acid in anhydrous THF (20 mL), 0.135 g (1.0 mmol) of HOBt and 1.0 mmol of (2S,3R)-2-Amino-3-hydroxy-N-octylbutanamide, (2S,3R)-2-Amino-3-hydroxy-N-dodecylbutanamide, (2S,3R)-2-Amino-3-hydroxy-N-tetradecylbutanamide, or (2S,3R)-2-Amino-3-hydroxy-N-octadecylbutanamide were added. After 5 min, 0.220 g (1.1 mmol) of EDC.HCl was added, and the pH of the solution was adjusted to 8-9 with 4-methylmorpholine. The mixture was stirred at 0° C. for 2 h and at room temperature overnight. On evaporation the residue was dissolved in 80 mL of ethyl acetate. The solution was washed successively with saturated sodium bicarbonate, 5% potassium bisulfate, and saturated sodium chloride, and the organic phase was separated and dried over anhydrous magnesium sulfate for 2 h. After filtration and evaporation under reduced pressure crude product was obtained and recrystallized using ethyl acetate to obtain compounds NZJU1g, NZJU2g, NZJU3g, and NZJU4g.

N-((2S,3R)-3-hydroxy-1-oxo-1-(octylamino)butan-2-yl)-2-thiophene carboxamide (NZJU1g) was obtained in a yield of 0.235 g (69.1%) as colorless powder. ¹H NMR (300 MHz, CDCl₃) δ 7.59 (d, J=3.5 Hz, 1H), 7.53 (d, J=5.0 Hz, 1H), 7.19-7.06 (m, 2H), 6.95 (s, 1H), 4.48 (s, 2H), 3.32-3.16 (m, 2H), 1.55-1.43 (m, 2H), 1.35-1.13 (m, 13H), 0.86 (t, J=6.7 Hz, 3H); ¹³C NMR (75 MHz, CDCl₃) δ 171.0, 162.9, 137.9, 130.9, 128.8, 127.9, 66.7, 57.2, 39.7, 31.8, 29.4, 29.2, 26.9, 22.6, 18.2, 14.1; ESI/MS (m/e) 339.10 [M−H]⁻; Anal. Calcd. For C₁₇H₂₈N₂O₃S: C, 59.97; H, 8.29; N, 8.23%. Found: C, 60.02; H, 8.37; N, 8.21%.

N-((2S,3R)-3-hydroxy-1-oxo-1-(dodecylamino)butan-2-yl)-2-thiophene carboxamide (NZJU2g) was obtained in a yield of 0.298 g (75.3%) as colorless powder. ¹H NMR (300 MHz, CDCl₃) δ7.59 (d, J=3.1 Hz, 1H), 7.54 (d, J=4.9 Hz, 1H), 7.16-6.98 (m, 2H), 6.88 (s, 1H), 4.55-4.37 (m, 2H), 3.33-3.13 (m, 2H), 1.56-1.41 (m, 2H), 1.37-1.07 (m, 21H), 0.88 (t, J=6.5 Hz, 3H); ¹³C NMR (75 MHz, CDCl₃) δ 170.8, 162.9, 137.9, 130.9, 128.9, 127.9, 66.8, 57.2, 39.7, 31.9, 29.6, 29.5, 29.4, 29.3, 26.9, 22.7, 18.2, 14.1; ESI/MS (m/e) 397 [M+H]⁺; Anal. Calcd. For C₂₁H₃₆N₂O₃S: C, 63.60; H, 9.15; N, 7.06%. Found: C, 63.57; H, 9.21; N, 7.01%.

N-((2S,3R)-3-hydroxy-1-oxo-1-(tetradecylamino)butan-2-yl)-2-thiophene carboxamide (NZJU3g) was obtained in a yield of 0.324 g (76.4%) as colorless powder. ¹H NMR (300 MHz, CDCl₃) δ 7.58 (d, J=3.7 Hz, 1H), 7.54 (d, J=4.9 Hz, 1H), 7.15-7.08 (m, 1H), 7.04 (s, 1H), 6.85 (s, 1H), 4.56-4.35 (m, 2H), 3.34-3.12 (m, 2H), 1.55-1.42 (m, 2H), 1.35-1.12 (m, 25H), 0.88 (t, J=6.6 Hz, 3H); ¹³C NMR (75 MHz, CDCl₃) δ 170.9, 162.9, 137.9, 130.9, 128.9, 127.9, 66.7, 57.2, 39.7, 31.9, 29.6, 29.5, 29.4, 29.3, 26.9, 22.7, 18.2, 14.1; ESI/MS (m/e) 425 [M+H]⁺; Anal. Calcd. For C₂₃H₄₀N₂O₃S: C, 65.05; H, 9.49; N, 6.60%. Found: C, 65.07; H, 9.54; N, 6.68%.

N-((2S,3R)-3-hydroxy-1-oxo-1-(octadecylamino)butan-2-yl)-2-thiophene carboxamide (NZJU4g) was obtained in a yield of 0.376 g (78.3%) as colorless powder. ¹H NMR (300 MHz, CDCl₃) δ 7.59 (d, J=3.3 Hz, 1H), 7.54 (d, J=4.8 Hz, 1H), 7.16-7.03 (m, 2H), 6.86 (s, 1H), 4.55-4.32 (m, 2H), 3.35-3.11 (m, 2H), 1.55-1.40 (m, 2H), 1.39-1.09 (m, 33H), 0.88 (t, J=5.6 Hz, 3H); ¹³C NMR (75 MHz, CDCl₃) δ 171.1, 162.9, 137.9, 130.9, 128.8, 127.9, 66.5, 56.9, 39.6, 31.9, 29.7, 29.6, 29.5, 29.4, 29.3, 26.9, 22.7, 18.2, 14.2; ESI/MS (m/e) 481.25 [M+H]⁺; Anal. Calcd. For C₂₇H₄₈N₂O₃S: C, 67.46; H, 10.06; N, 5.83%. Found: C, 67.37; H, 10.11; N, 5.78%.

### Example 12

### Condensation of NZJU1-NZJU4 with Benzofuran-2-carboxylic Acid

At 0° C., to a solution of 1.0 mmol of benzofuran-2-carboxylic acid in anhydrous THF (20 mL), 0.135 g (1.0 mmol) of HOBt and 1.0 mmol of (2S,3R)-2-Amino-3-hydroxy-N-octylbutanamide, (2S,3R)-2-Amino-3-hydroxy-N-dodecylbutanamide, (2S,3R)-2-Amino-3-hydroxy-N-tetradecylbutanamide, or (2S,3R)-2-Amino-3-hydroxy-N-octadecylbutanamide were added. After 5 min, 0.220 g (1.1 mmol) of EDC.HCl was added, and the pH of the solution was adjusted to 8-9 with 4-methylmorpholine. The mixture was stirred at 0° C. for 2 h and at room temperature overnight. On evaporation the residue was dissolved in 80 mL of ethyl acetate. The solution was washed successively with saturated sodium bicarbonate, 5% potassium bisulfate, and saturated sodium chloride, and the organic phase was separated and dried over anhydrous magnesium sulfate for 2 h. After filtration and evaporation under reduced pressure crude product was obtained and recrystallized using ethyl acetate to obtain compounds NZJU1 h, NZJU2h, NZJU3h, and NZJU4h.

N-((2S,3R)-3-hydroxy-1-oxo-1-(octylamino)butan-2-yl)-benzofuran-2-carboxamide (NZJU1h) was obtained in a yield of 0.248 g (66.3%) as colorless powder. ¹H NMR (300 MHz, CDCl₃) δ 7.70 (d, J=6.9 Hz, 1H), 7.66 (d, J=7.8 Hz, 1H), 7.53 (d, J=8.3 Hz, 1H), 7.48 (s, 1H), 7.46-7.37 (m, 1H), 7.33-7.27 (m, 1H), 6.96 (s, 1H), 4.61-4.42 (m, 2H), 3.36-3.12 (m, 2H), 1.57-1.41 (m, 2H), 1.37-1.06 (m, 13H), 0.83 (t, J=6.7 Hz, 3H); ¹³C NMR (75 MHz, CDCl₃) δ 170.3, 159.8, 155.0, 147.9, 127.3, 123.8, 122.7, 112.0, 111.2, 66.8, 57.2, 39.7, 31.8, 29.4, 29.2, 26.9, 22.6, 18.2, 14.1; ESI/MS (m/e) 409.15 [M+Cl]⁻; Anal. Calcd. For C₂₁H₃₀N₂O₄: C, 67.35; H, 8.07; N, 7.48%. Found: C, 67.37; H, 8.13; N, 7.39%.

N-((2S,3R)-3-hydroxy-1-oxo-1-(dodecylamino)butan-2-yl)-benzofuran-2-carboxamide (NZJU2h) was obtained in a yield of 0.276 g (64.2%) as colorless powder. ¹H NMR (300 MHz, CDCl₃) δ 7.76-7.61 (m, 2H), 7.53 (d, J=8.3 Hz, 1H), 7.48 (s, 1H), 7.47-7.38 (m, 1H), 7.31 (d, J=7.2 Hz, 1H), 6.95 (s, 1H), 4.64-4.41 (m, 2H), 3.39-3.09 (m, 2H), 1.59-1.41 (m, 2H), 1.39-1.02 (m, 21H), 0.87 (t, J=6.6 Hz, 3H); ¹³C NMR (75 MHz, CDCl₃) δ 170.6, 159.8, 155.0, 147.8, 127.4, 123.9, 122.7, 112.1, 111.3, 66.6, 56.8, 39.7, 31.9, 29.6, 29.6, 29.5, 29.4, 29.3, 26.9, 22.7, 18.2, 14.1; ESI/MS (m/e) 431.10 [M+H]⁺; Anal. Calcd. For C₂₅H₃₈N₂O₄: C, 69.74; H, 8.90; N, 6.51%. Found: C, 69.78; H, 8.76; N, 6.44%.

N-((2S,3R)-3-hydroxy-1-oxo-1-(tetradecylamino)butan-2-yl)-benzofuran-2-carboxamide (NZJU3h) was obtained in a yield of 0.323 g (70.5%) as colorless powder. ¹H NMR (300 MHz, CDCl₃) δ 7.74-7.60 (m, 2H), 7.54 (d, J=8.3 Hz, 1H), 7.50 (s, 1H), 7.44 (t, J=7.6 Hz, 1H), 7.31 (t, J=7.5 Hz, 1H), 6.85 (s, 1H), 4.61-4.44 (m, 2H), 3.37-3.09 (m, 2H), 1.58-1.40 (m, 2H), 1.41-1.01 (m, 25H), 0.88 (t, J=6.5 Hz, 3H); ¹³C NMR (75 MHz, CDCl₃) δ 170.5, 159.7, 155.0, 147.8, 127.3, 123.8, 122.7, 112.1, 111.2, 66.6, 56.9, 39.7, 31.9, 29.7, 29.5, 29.4, 29.3, 26.9, 22.7, 18.2, 14.1; ESI/MS (m/e) 459.15 [M+H]⁺; Anal. Calcd. For C₂₇H₄₂N₂O₄: C, 70.71; H, 9.23; N, 6.11%. Found: C, 70.59; H, 9.29; N, 6.18%.

N-((2S,3R)-3-hydroxy-1-oxo-1-(octadecylamino)butan-2-yl)-benzofuran-2-carboxamide (NZJU4h) was obtained in a yield of 0.362 g (70.4%) as colorless powder. ¹H NMR (300 MHz, CDCl₃) δ 7.71-7.62 (m, 2H), 7.54 (d, J=8.3 Hz, 1H), 7.50 (s, 1H), 7.48-7.40 (m, 1H), 7.30 (t, J=7.2 Hz, 1H), 6.87 (s, 1H), 4.59-4.44 (m, 2H), 3.35-3.15 (m, 2H), 1.57-1.43 (m, 2H), 1.36-1.11 (m, 33H), 0.88 (t, J=6.6 Hz, 3H); ¹³C NMR (75 MHz, CDCl₃) δ 170.4, 159.7, 155.0, 147.8, 127.3, 123.8, 122.7, 112.0, 111.2, 66.6, 56.9, 39.7, 31.9, 29.7, 29.6, 29.5, 29.4, 29.3, 26.9, 22.7, 18.2, 14.1; ESI/MS (m/e) 515.25 [M+H]⁺; Anal. Calcd. For C₃₁H₅₀N₂O₄: C, 72.33; H, 9.79; N, 5.44%. Found: C, 72.31; H, 9.81; N, 5.43%.

### Example 13

### Condensation of NZJU2-NZJU4 with 1H-indole-2-carboxylic Acid

At 0° C., to a solution of 1.0 mmol of 1H-indole-2-carboxylic acid in anhydrous THF (20 mL), 0.135 g (1.0 mmol) of HOBt and 1.0 mmol of (2S,3R)-2-Amino-3-hydroxy-N-dodecylbutanamide, (2S,3R)-2-Amino-3-hydroxy-N-tetradecylbutanamide, or (2S,3R)-2-Amino-3-hydroxy-N-octadecylbutanamide were added. After 5 min, 0.220 g (1.1 mmol) of EDC.HCl was added, and the pH of the solution was adjusted to 8-9 with 4-methylmorpholine. The mixture was stirred at 0° C. for 2 h and at room temperature overnight. On evaporation the residue was dissolved in 80 mL of ethyl acetate. The solution was washed successively with saturated sodium bicarbonate, 5% potassium bisulfate, and saturated sodium chloride, and the organic phase was separated and dried over anhydrous magnesium sulfate for 2 h. After filtration and evaporation under reduced pressure crude product was obtained and recrystallized using ethyl acetate to obtain compounds NZJU2i, NZJU3i, and NZJU4i.

N-((2S,3R)-3-hydroxy-1-oxo-1-(dodecylamino)butan-2-yl)-indole-2-carboxamide (NZJU2i) was obtained in a yield of 0.292 g (68.1%) as colorless powder. ¹H NMR (300 MHz, CDCl₃) δ 9.69 (s, 1H), 7.65 (d, J=8.0 Hz, 1H), 7.49 (d, J=7.6 Hz, 1H), 7.40 (d, J=8.2 Hz, 1H), 7.30 (d, J=7.3 Hz, 1H), 7.14 (t, J=7.4 Hz, 1H), 7.08 (s, 1H), 6.88 (t, J=5.3 Hz, 1H), 4.67-4.44 (m, 2H), 3.40-3.06 (m, 2H), 1.58-1.38 (m, 2H), 1.37-1.05 (m, 21H), 0.88 (t, J=6.6 Hz, 3H); ¹³C NMR (75 MHz, CDCl₃) δ 170.7, 162.7, 136.8, 129.7, 127.5, 124.9, 122.2, 120.8, 112.0, 104.5, 67.0, 57.5, 39.8, 31.9, 30.3, 29.6, 29.5, 29.4, 29.3, 26.9, 22.7, 18.4, 14.1; ESI/MS (m/e) 430 [M+H]⁺; Anal. Calcd. For C₂₅H₃₉N₃O₃: C, 69.90; H, 9.15; N, 9.78%. Found: C, 69.87; H, 9.07; N, 9.75%.

N-((2S,3R)-3-hydroxy-1-oxo-1-(tetradecylamino)butan-2-yl)-indole-2-carboxamide (NZJU3i) was obtained in a yield of 0.335 g (73.3%) as colorless powder. ¹H NMR (300 MHz, CDCl₃) δ 9.61 (s, 1H), 7.65 (d, J=7.9 Hz, 1H), 7.46-7.40 (m, 2H), 7.30 (d, J=7.3 Hz, 1H), 7.15 (t, J=7.5 Hz, 1H), 7.07 (s, 1H), 6.86 (t, J=5.3 Hz, 1H), 4.58-4.52 (m, 2H), 3.40-3.06 (m, 2H), 1.55-1.40 (m, 2H), 1.39-1.10 (m, 25H), 0.87 (t, J=6.8 Hz, 3H); ¹³C NMR (75 MHz, CDCl₃) δ 170.7, 162.8, 136.9, 129.8, 127.5, 124.9, 122.2, 120.8, 112.1, 104.7, 67.1, 57.7, 39.8, 31.9, 30.3, 29.7, 29.4, 29.3, 26.9, 22.7, 18.5, 14.1; ESI/MS (m/e) 458 [M+H]⁺; Anal. Calcd. For C₂₇H₄₃N₃O₃: C, 70.86; H, 9.47; N, 9.18%. Found: C, 70.91; H, 9.54; N, 9.21%.

N-((2S,3R)-3-hydroxy-1-oxo-1-(octadecylamino)butan-2-yl)-indole-2-carboxamide (NZJU4i) was obtained in a yield of 0.367 g (71.5%) as colorless powder. ¹H NMR (300 MHz, CDCl₃) δ 9.40 (s, 1H), 7.67 (d, J=7.9 Hz, 1H), 7.46-7.40 (m, 2H), 7.32 (d, J=6.8 Hz, 1H), 7.16 (t, J=7.4 Hz, 1H), 7.05 (s, 1H), 6.82 (s, 1H), 4.58-4.50 (m, 2H), 3.34-3.14 (m, 2H), 1.55-1.40 (m, 2H), 1.39-1.10 (m, 33H), 0.88 (t, J=6.2 Hz, 3H); ¹³C NMR (75 MHz, DMSO) δ 170.4, 161.5, 137.0, 131.8, 127.5, 123.9, 122.0, 120.2, 112.7, 104.0, 67.2, 59.5, 40.0, 31.8, 29.5, 29.3, 28.6, 26.8, 22.6, 20.7, 14.4; ESI/MS (m/e) 512.30 [M−H]⁻; Anal. Calcd. For C₃₁H₅₁N₃O₃: C, 72.47; H, 10.01; N, 8.18%. Found: C, 72.39; H, 9.94; N, 8.07%.

### Example 14

### Condensation of NZJU2-NZJU4 with 1-methyl-1H-indole-2-carboxylic Acid

At 0° C., to a solution of 1.0 mmol of 1-methyl-1H-indole-2-carboxylic acid in anhydrous THF (20 mL), 0.135 g (1.0 mmol) of HOBt and 1.0 mmol of (2S,3R)-2-Amino-3-hydroxy-N-dodecylbutanamide, (2S,3R)-2-Amino-3-hydroxy-N-tetradecylbutanamide, or (2S,3R)-2-Amino-3-hydroxy-N-octadecylbutanamide were added. After 5 min, 0.220 g (1.1 mmol) of EDC.HCl was added, and the pH of the solution was adjusted to 8-9 with 4-methylmorpholine. The mixture was stirred at 0° C. for 2 h and at room temperature overnight. On evaporation the residue was dissolved in 80 mL of ethyl acetate. The solution was washed successively with saturated sodium bicarbonate, 5% potassium bisulfate, and saturated sodium chloride, and the organic phase was separated and dried over anhydrous magnesium sulfate for 2 h. After filtration and evaporation under reduced pressure crude product was obtained and recrystallized using ethyl acetate to obtain compounds NZJU2j, NZJU3j, and NZJU4j.

N-((2S,3R)-3-hydroxy-1-oxo-1-(dodecylamino)butan-2-yl)-(N-methyl) indole-2-carboxamide (NZJU2j) was obtained in a yield of 0.298 g (67.3%) as colorless powder. ¹H NMR (300 MHz, CDCl₃) δ 8.07-7.96 (m, 1H), 7.75 (s, 1H), 7.45-7.29 (m, 3H), 7.10 (s, 1H), 7.04 (s, 1H), 4.60-4.48 (m, 2H), 3.84 (s, 3H), 3.32-3.18 (m, 2H), 1.55-1.42 (m, 2H), 1.37-1.10 (m, 21H), 0.88 (t, J=6.6 Hz, 3H). ¹³C NMR (75 MHz, CDCl₃) δ 171.4, 165.8, 137.3, 132.7, 125.5, 122.8, 121.9, 120.4, 110.0, 109.8, 66.9, 56.9, 39.6, 33.3, 31.9, 29.6, 29.5, 29.4, 29.3, 26.9, 22.7, 18.2, 14.1; ESI/MS (m/e) 444 [M+H]⁺; Anal. Calcd. For C₂₆H₄₁N₃O₃: C, 70.39; H, 9.32; N, 9.47%. Found: C, 70.21; H, 9.48; N, 9.39%.

N-((2S,3R)-3-hydroxy-1-oxo-1-(tetradecylamino)butan-2-yl)-(N-methyl)indole-2-carboxamide (NZJU3j) was obtained in a yield of 0.326 g (69.2%) as colorless powder. ¹H NMR (300 MHz, CDCl₃) δ 8.09-7.96 (m, 1H), 7.74 (s, 1H), 7.43-7.28 (m, 3H), 7.16 (s, 1H), 7.09 (s, 1H), 4.66-4.45 (m, 2H), 3.83 (s, 3H), 3.36-3.12 (m, 2H), 1.59-1.40 (m, 2H), 1.37-1.06 (m, 25H), 0.88 (t, J=6.5 Hz, 3H). ¹³C NMR (75 MHz, MeOD) δ 171.3, 165.8, 137.2, 132.7, 125.6, 122.7, 121.8, 120.5, 110.0, 109.7, 67.2, 57.2, 39.7, 33.2, 31.9, 29.7, 29.5, 29.4, 29.3, 27.0, 22.7, 18.3, 14.1; ESI/MS (m/e) 494 [M+Na]⁺; Anal. Calcd. For C₂₈H₄₅N₃O₃: C, 71.30; H, 9.62; N, 8.91%. Found: C, 71.21; H, 9.76; N, 8.87%.

N-((2S,3R)-3-hydroxy-1-oxo-1-(octadecylamino)butan-2-yl)-(N-methyl)indole-2-carboxamide (NZJU4j) was obtained in a yield of 0.358 g (67.9%) as colorless powder. ¹H NMR (300 MHz, CDCl₃) δ 8.08-7.96 (m, 1H), 7.74 (s, 1H), 7.42-7.29 (m, 3H), 7.16 (s, 1H), 7.13-7.04 (m, 1H), 4.71-4.41 (m, 2H), 3.83 (s, 3H), 3.36-3.13 (m, 2H), 1.57-1.39 (m, 2H), 1.37-1.07 (m, 33H), 0.88 (t, J=6.4 Hz, 3H). ¹³C NMR (75 MHz, CDCl₃) δ 171.2, 165.8, 137.2, 132.7, 125.6, 122.7, 121.8, 120.5, 110.0, 109.7, 67.2, 57.3, 39.7, 33.2, 31.9, 29.7, 29.6, 29.4, 29.3, 27.0, 22.7, 18.3, 14.1; ESI/MS (m/e) 528 [M+H]⁺; Anal. Calcd. For C₃₂H₅₃N₃O₃: C, 72.82; H, 10.12; N, 7.96%. Found: C, 72.78; H, 10.17; N, 8.06%.

### Example 15

### Cell Viability Assays

Cell survival after exposure to the sphingolipid metabolite mimetics was examined by MTT cytotoxicity assay. HepG2, A431, HeLa, MCF-7 and SA cells (7000 cells/well) were seeded in each well of a 96-well plate for 24 h. New medium was added to give final concentrations of 0 to 200 μM of the sphingolipid metabolite mimetics. Then, 50 μl of 1 mg/ml 3-(4,5-dimethylthiazol-2-yl)-2,5-diphenyltetrazolium bromide (MTT, Sigma-Aldrich Chemical) was added to each well for 4 h incubation at 37° C. The purple formazan formed was then solubilized by DMSO and absorbance at 570 nm was read by a microplate reader (Molecular Devices, Sunnyvale, Calif.).

The measured IC₅₀ values (μM) are reported in Table 1. Several of the sphingolipid metabolite mimetics exhibit IC₅₀ values that are below 30 μM, many below 20 μM. NZJU3i presents IC₅₀ values below 20 μM for all of the cell lines tested. NZJU2e, NZJU3e, NZJU3f, NZJU2h, and NZJU3h exhibit IC₅₀ values below 30 μM for all five cell lines tested.

TABLE 1
The cytotoxicity data (IC₅₀ μM) of ceramide analogs against HepG2,
A431, SA, MCF-7, SiHa, SW620, and SW480.

| | | Squamous | Pancreatic | Breast | Ovarian | | |
|---|---|---|---|---|---|---|---|
| | Liver Cancer | Carcinoma | Cancer | Cancer | Cancer | Colon Cancer | Colon Cancer |
| | HepG2 | A431 | SA | MCF-7 | SiHa | SW620 | SW480 |
| NZJU4 | 44.52 ± 3.49 | 21.32 ± 1.72 | | 27.42 ± 1.8 | 39.54 ± 0.88 | 21.37 ± 1.24 | 27.525 ± 3.03 |
| NZJU9 | 72.46 ± 9.63 | 26.73 ± 0.84 | 49.07 ± 4.05 | 50.26 ± 1.06 | 34.22 ± 0.13 | 40.54 ± 0.76 | 44.49 ± 4.97 |
| NZJU10 | >400 | 171.45 ± 12.08 | 189.28 ± 12.18 | 37.1 ± 4.11 | >200 | 200 | >200 |
| NZJU11 | >400 | >200 | 184.37 ± 1.9 | 27.76 ± 0.14 | >200 | 200 | >200 |
| NZJU12 | 284.22 ± 8.30 | 187.13 ± 2.41 | >200 | 79.63 ± 2.34 | >200 | 200 | 195.61 |
| NZJU13 | 146.86 ± 22.28 | 92.44 ± 2.64 | 93.49 ± 3.13 | 67.49 ± 4.88 | 64.32 ± 1.68 | 87.5 ± 7.58 | 125.01 ± 16.05 |
| NZJU14 | >400 | 190.28 ± 6.86 | 148 ± 11.63 | 104.63 ± 5.03 | 127.1 ± 7.41 | 200 | 110.34 ± 5.03 |
| NZJU15 | 42.29 ± 3.73 | 19.93 ± 0.45 | 52.55 ± 6.02 | 21.38 ± 2.4 | 13.35 ± 1.05 | | 24.39 ± 0.7 |
| NZJU16 | >400 | 41.18 ± 0.69 | 75.45 ± 6.24 | 36.57 ± 8.86 | 63.92 ± 6.01 | 200 | 67.21 ± 7.42 |
| NZJU17 | 54.21 ± 3.95 | 118.49 ± 9.09 | 56.04 ± 7.43 | 24.77 ± 6.75 | >200 | 200 | 200 |
| NZJU18 | >400 | 113.05 ± 7.87 | >400 | 31.71 ± 6.55 | 38.22 ± 2.52 | 200 | 36.47 ± 0.23 |
| NZJU19 | >400 | >200 | >200 | >400 | >200 | 200 | >200 |
| NZJU24 | >200 | >200 | 154.82 ± 1.16 | >200 | >200 | 200 | >200 |
| NZJU25 | 150.18 ± 4.37 | 64.86 ± 4.87 | 80.87 ± 1.9 | 64.57 ± 4.54 | 35.82 ± 0.68 | 200 | 86.89 |
| NZJU26 | 127.61 ± 9.04 | 177.08 ± 7.17 | 47.09 ± 0.54 | 39.22 ± 2.21 | 35.94 ± 2.64 | 200 | 123.08 |
| NZJU27 | 22.61 ± 2.89 | 23.41 ± 3.48 | 19.97 ± 0.74 | 15.5 ± 2.3 | 13.6 ± 1.02 | 22.02 ± 2.86 | 20 |
| NZJU28 | 21.11 ± 1.92 | 15.47 ± 2.95 | 11.52 ± 0.97 | 6.81 ± 1.73 | 4.8 ± 0.29 | 21.965 ± 0.71 | 20 |
| NZJU29 | 57.57 ± 2.69 | 31.78 ± 1.93 | 54.91 ± 3.90 | 37.33 ± 1.82 | 65.85 ± 6.45 | 103 ± 7.48 | 60 |
| NZJU30 | 65.69 ± 2.63 | 62.76 ± 7.56 | 51.93 ± 0.42 | 50.53 ± 5.65 | 56.62 ± 4.91 | 65.79 ± 4.14 | 58.8 |
| NZJU31 | 28.56 ± 3.77 | 23.04 ± 3.72 | 24.41 ± 3.60 | 22.79 ± 3.94 | 21.6 ± 2.26 | 30.4 ± 2.09 | 23.64 |
| NZJU32 | 96.94 ± 6.77 | 44.13 ± 2.02 | 49.14 ± 4.72 | 30.78 ± 0.67 | 51.17 ± 3.46 | 136.26 ± 13.29 | 30.72 |
| NZJU33 | 30.11 ± 3.84 | 16.61 ± 2.12 | 23.28 ± 3.33 | 30.43 ± 2.8 | 23.85 ± 3.65 | 29.19 ± 0.57 | 30.21 |
| NZJU34 | 88.4 ± 15.53 | 26.45 ± 4.03 | 21.22 ± 3.78 | 36.07 ± 0.27 | 63 ± 0.25 | 78.44 ± 11.128 | 70.31 |
| NZJU35 | 91.96 ± 7.76 | 33.3 ± 3.32 | 53.49 ± 5.32 | 25.73 ± 0.47 | 116.6 ± 3.9 | 200 | 88.97 |
| NZJU36 | 22.85 ± 1.18 | 20.04 ± 0.9 | 10.49 ± 1.31 | 20.89 ± 0.54 | 12.85 ± 2.07 | 21.32 ± 1.28 | 21.41 |
| NZJU37 | 23.38 ± 1.19 | 20.09 ± 0.16 | 9.61 ± 0.56 | 23.38 ± 1.19 | 14.23 ± 1.84 | 26.7 ± 2.48 | 22.75 |
| NZJU38 | >200 | 78.79 ± 6.99 | 161.96 ± 4.55 | 126.36 ± 7.19 | >200 | | >200 |
| NZJU39 | 136.48 ± 5.92 | 61.33 ± 4.09 | 34.62 ± 4.17 | 111.47 ± 4.66 | 112.45 ± 2.43 | 200 | >200 |
| NZJU40 | 192.16 ± 11.09 | 64.5 ± 0.78 | 64.65 ± 0.5 | 91.84 ± 4.21 | 124.01 ± 8.78 | 200 | 194.2 |
| NZJU41 | 101.68 ± 4.07 | 58.24 ± 0.11 | 40.44 ± 2.16 | 148.86 ± 3.24 | 123.22 ± 2.5 | 200 | 158.12 |
| NZJU42 | 55.55 ± 0.64 | 36.04 ± 3.36 | 25.59 ± 4.41 | 35.06 ± 0.2 | 41.41 ± 5.96 | 61.35 ± 0.27 | 62.71 |
| NZJU43 | 88.19 ± 1.92 | 26.87 ± 2.81 | 22.91 ± 2.59 | 63.21 ± 4.73 | 65.99 ± 5.48 | 67.75 ± 0.099 | |
| NZJU44 | 149.26 ± 8.7 | 32.15 ± 4.6 | 60.93 ± 4.29 | 72.46 ± 3.37 | 91.85 ± 0.59 | 136.32 ± 42.57 | |
| NZJU45 | 81.26 ± 9.13 | 52.74 ± 3.82 | 40.78 ± 3.63 | 47.22 ± 7.3 | 66.48 ± 2.5 | 200 | |
| NZJU46 | N/A | 14.58 ± 0.76 | 10.98 ± 1.39 | 10.69 ± 0.98 | 10.98 ± 1.39 | 18.74 ± 3.81 | |
| NZJU47 | 11.97 ± 2.38 | 10.31 ± 2.78 | 12.28 ± 2.75 | | 21.25 ± 0.01 | | 10.19 ± 0.73 |
| NZJU48 | 10.51 ± 0.35 | 12.25 ± 1.90 | 12.11 ± 2.77 | | 14.20 ± 1.80 | | 11.84 ± 1.46 |
| NZJU49 | 89.42 ± 0.27 | 80.97 ± 11.42 | 35.65 ± 0.27 | | 29.56 ± 1.48 | | 34.76 ± 0.56 |
| NZJU50 | >200 | 168.74 ± 14.66 | 31.3 ± 1.14 | | >200 | | 35.99 ± 2.87 |
| NZJU51 | 34.45 ± 2.34 | 13.68 ± 1.73 | 25.65 ± 0.42 | | 26.52 ± 2.33 | | 34.19 ± 1.36 |
| NZJU52 | >200 | 60.98 ± 1.82 | 36.37 ± 0.19 | | 112.98 ± 12.02 | | 35.96 ± 3.23 |
| NZJU53 | 42.96 ± 4.04 | 32.98 ± 3.55 | 37.06 ± 1.17 | | 27.40 ± 2.07 | | 58.13 ± 4.37 |
| NZJU54 | 23.6 ± 1.16 | 24.39 ± 1.27 | 24.23 ± 0.83 | | 30.13 ± 3.01 | | 33.25 ± 1.08 |
| NZJU55 | 73.69 ± 5.21 | 85.82 ± 1.73 | 84.81 ± 4.34 | | 48.07 ± 4.67 | | 160.335 ± 5.86 |
| NZJU56 | 25.78 ± 0.52 | 25.19 ± 0.99 | 27.23 ± 0.99 | | 31.59 ± 4.56 | | 39.9 ± 2.54 |
| NZJU57 | 10.92 ± 0.54 | 11.99 ± 1.87 | 10.89 ± 1.25 | | 19.23 ± 2.14 | | 11.78 ± 1.38 |
| NZJU58 | 75.76 ± 9.46 | 34.03 ± 1.03 | 47.88 ± 5.2 | | 29.78 ± 2.23 | | >200 |
| NZJU59 | 25.88 ± 1.21 | 21.99 ± 0.86 | 20.1 ± 1.65 | | 30.94 ± 1.28 | | 41.04 ± 2.25 |
| NZJU60 | 21.57 ± 2.05 | 21.97 ± 0.56 | 11.56 ± 2.2 | | 20.37 ± 2.47 | | 11.82 ± 1.25 |
| NZJU61 | >200 | >200 | >200 | | >200 | | >200 |
| NZJU62 | 11.38 ± 1.11 | 19.57 ± 2.16 | 10.21 ± 0.3 | | 11.10 ± 1.39 | | 5.76 ± 1.02 |
| NZJU63 | 19.83 ± 3.12 | 23.66 ± 1.46 | 21.23 ± 0.81 | | 19.90 ± 3.31 | | 69.06 ± 9.67 |
| NZJU64 | 29.92 ± 2.81 | 23.07 ± 2.97 | 33.7 ± 1.28 | | 24.56 ± 2.04 | | 47.77 ± 3.43 |

### Example 16

### Apoptosis Assays

Detection of apoptotic cells by fluorescence staining: MES-SA cells treated with or without sphingolipid metabolite mimetics at doses of their IC50 for 8 h were washed with PBS, and then fixed in ice-cold 70% ethanol for 15 min. The cells were then washed with PBS and stained with Hoechst 33342 (from sigma chemical) for 20 min in the dark. Morphologic changes were analyzed under a fluorescence microscope. The experiment was repeated three times.

### Example 17

### Drug Sensitivity Assays

Cell survival after exposure to the anti-tumor agents was examined by MTT cytotoxicity assay. Cells were seeded in each well of a 96-well plate for 24 h. After incubation with various concentrations of compounds, TRAIL, or both for 48 h, the medium was discarded. Then, 50 μl of 1 mg/ml 3-(4,5-dimethylthiazol-2-yl)-2,5-diphenyltetrazolium bromide (MTT, Sigma-Aldrich Chemical) was added to each well for 4 h incubation at 37° C. The purple formazan formed was then solubilized by DMSO and absorbance at 570 nm was read by a microplate reader (Molecular Devices, Sunnyvale, Calif.).

TABLE 2
Drug sensitivity data for TRAIL in SW620

| | | | NZJU alone | | combine | | Enhanced | |
|---|---|---|---|---|---|---|---|---|
| SW620 | Dose | | mean | | mean | | mean | |
| Drugs | (μM) | | value | SD | value | SD | value | SD |
| TRAIL | 100 | ng/ml | 0.177 | 0.043 | | | | |
| NZJU13 | 22 | | 0.126 | 0.034 | 0.520 | 0.100 | 0.321 | 0.039 |
| (NZJU2b) | | | | | | | | |
| NZJU16 | 25 | | 0.376 | 0.034 | 0.607 | 0.119 | 0.304 | 0.121 |
| (NZJU2a) | | | | | | | | |
| NZJU30 | 33 | | −0.038 | 0.065 | 0.493 | 0.127 | 0.375 | 0.177 |
| (NZJU2f) | | | | | | | | |
| NZJU34 | 19 | | 0.315 | 0.084 | 0.664 | 0.101 | 0.275 | 0.121 |
| (NZJU3g) | | | | | | | | |
| NZJU41 | 100 | | 0.551 | 0.025 | 0.790 | 0.055 | 0.169 | 0.144 |
| (NZJU1c) | | | | | | | | |
| NZJU44 | 34 | | 0.563 | 0.009 | 0.853 | 0.009 | 0.128 | 0.112 |
| (NZJU1g) | | | | | | | | |
| NZJU45 | 25 | | 0.205 | 0.014 | 0.516 | 0.072 | 0.309 | 0.019 |
| (NZJU1h) | | | | | | | | |

The SW620 data is presented in Table 2. SW620 cells exhibit sensitivity to the different sphingolipid metabolite mimetics. NZJU1c and NZJU1g demonstrate sensitivities when used alone of greater than 0.5. NZJU2a, NZJU2b, NZJU2f, NZJU3g, and NZJU1h are observed to sensitize SW620 cells to TRAIL, demonstrating enhancements greater than 0.25. SW620 cells exhibit very little sensitivity to NZJU2f when provided alone, but an enhancement of 0.375 is observed when provided in combination with TRAIL.

The MCF-7 data is presented in Table 3. MCF-7 cells exhibit sensitivities ranging from 0.021 to 0.625 to the sphingolipid metabolite mimetics when provided alone. NZJU1g is observed to be efficient at killing MCF-7 cells when provided alone. NZJU2b, NZJU3g, and NZJU1h are observed to be efficient at sensitizing MCF-7 cells to TRAIL.

TABLE 3
Drug sensitivity data for TRAIL in MCF-7

| MCF-7 | | | NZJU alone | | combine | | Enhanced | |
|---|---|---|---|---|---|---|---|---|
| Drugs | Dose (μM) | | mean value | SD | mean value | SD | mean value | SD |
| TRAIL | 100 | ng/ml | 0.118 | 0.008 | | | | |
| NZJU13 (NZJU2b) | 22 | | 0.148 | 0.026 | 0.582 | 0.006 | 0.321 | 0.064 |
| NZJU16 (NZJU2a) | 25 | | 0.479 | 0.178 | 0.741 | 0.016 | 0.197 | 0.203 |
| NZJU30 (NZJU2f) | 33 | | 0.002 | 0.002 | 0.274 | 0.095 | 0.157 | 0.011 |
| NZJU34 (NZJU3g) | 19 | | 0.444 | 0.177 | 0.759 | 0.053 | 0.263 | 0.084 |
| NZJU41 (NZJU1c) | 100 | | 0.347 | 0.038 | 0.625 | 0.060 | 0.113 | 0.014 |
| NZJU44 (NZJU1g) | 34 | | 0.625 | 0.058 | 0.717 | 0.119 | 0.042 | 0.041 |
| NZJU45 (NZJU1h) | 25 | | 0.021 | 0.029 | 0.538 | 0.085 | 0.382 | 0.118 |

The HELA cell data is presented in Table 4. Sphingolipid metabolite mimetics are observed to be efficient at inducing apoptosis in HELA cells. NZJU2a, NZJU3g, NZJU1c, and NZJU1g demonstrate sensitivities for HELA cells greater than 0.2 when provided alone. When provided in combination with TRAIL, NZJU2b, NZJU2a, NZJU3g, NZJU1c, NZJU1g, and NZJU1h are observed to sensitize HELA cells to TRAIL-induced apoptosis.

TABLE 4
Drug sensitivity data for TRAIL in HELA

| HELA | | | NZJU alone | | combine | | Enhanced | |
|---|---|---|---|---|---|---|---|---|
| Drugs | Dose (μM) | | mean value | SD | mean value | SD | mean value | SD |
| TRAIL | 100 | ng/ml | 0.036 | 0.050 | | | | |
| NZJU13 (NZJU2b) | 22 | | 0.146 | 0.007 | 0.435 | 0.117 | 0.254 | 0.074 |
| NZJU16 (NZJU2a) | 25 | | 0.264 | 0.086 | 0.597 | 0.050 | 0.298 | 0.086 |
| NZJU30 (NZJU2f) | 33 | | 0.057 | 0.016 | 0.195 | 0.021 | 0.075 | 0.007 |
| NZJU34 (NZJU3g) | 19 | | 0.334 | 0.083 | 0.744 | 0.009 | 0.375 | 0.023 |
| NZJU41 (NZJU1c) | 100 | | 0.362 | 0.002 | 0.819 | 0.050 | 0.421 | 0.002 |
| NZJU44 (NZJU1g) | 34 | | 0.218 | 0.288 | 0.315 | 0.022 | 0.357 | 0.101 |
| NZJU45 (NZJU1h) | 25 | | 0.173 | 0.039 | 0.432 | 0.015 | 0.223 | 0.026 |

1. (canceled)

2. A compound having a structure according to Formula I or Formula II 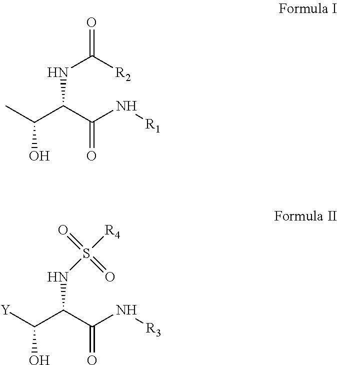
or a derivative thereof, wherein
Y is hydrogen, methyl, alkyl, or substituted alkyl;
R₁ and R₃ are alkyl or substituted alkyl; and
R₂ and R₄ are alkyl, substituted alkyl, aryl, or substituted aryl.

3. The compound of claim 2 having a structure according to Formula I, wherein
R₁ is a C₁₀-C₂₀ linear alkyl, and
R₂ is selected from the group consisting of phenyl, 2-furanyl, 2-thiophenyl, 1,3-pentadienyl, 2-phenylethenyl, isoquinoline, indole, methyl indole, benzofuranyl, naphthalene, hydroxynaphthalene, and cyclohex-4-ene-1,2,3-triol.

4. The compound of claim 2 having a structure according to Formula II, wherein
Y is methyl,
R₃ is a C₁₀-C₂₀ linear alkyl, and
R₄ is selected from the group consisting of phenyl, 2-furanyl, 2-thiophenyl, 1,3-pentadienyl, 2-phenylethenyl, isoquinoline, indole, methyl indole, benzofuranyl, naphthalene, hydroxynaphthalene, and cyclohex-4-ene-1,2,3-triol.

5. The compound of claim 2 selected from the group consisting of (S)-2-Amino-3-hydroxy-N-dodecylpropanamide, (S)-2-Amino-3-hydroxy-N-dodecylpropanamide, (2S,3R)-2-Amino-3-hydroxy-N-octadecylbutanamide, (2E,4E)-N-((2S,3R)-3-hydroxy-1-oxo-1-(dodecylamino)butan-2-yl)-2,4-hexadienoic acid amide, (2E,4E)-N-((2S,3R)-3-hydroxy-1-oxo-1-(dodecylamino)butan-2-yl)-2,4-hexadienoic acid amide, (2E,4E)-N-((2S,3R)-3-hydroxy-1-oxo-1-(octadecylamino)butan-2-yl)-2,4-hexadienoic acid amide, (2E,4E)-N-((2S,3R)-3-hydroxy-1-oxo-1-(octadecylamino)butan-2-yl)-2,4-hexadienoic acid amide, N-((2S,3R)-3-hydroxy-1-oxo-1-(tetradecylamino)butan-2-yl)-Furan-2-carboxamide, N-((2S,3R)-3-hydroxy-1-oxo-1-(octadecylamino)butan-2-yl)-Furan-2-carboxamide, N-((2S,3R)-3-hydroxy-1-oxo-1-(dodecylamino)butan-2-yl)-benzamide, N-((2S,3R)-3-hydroxy-1-oxo-1-(tetradecylamino)butan-2-yl)-benzamide, N-((2S,3R)-3-hydroxy-1-oxo-1-(octadecylamino)butan-2-yl)-benzamide, N-((2S,3R)-3-Hydroxy-1-oxo-1-(benzamino)butan-2-yl)-tetradecyl acid amide, (E)-N-((2S,3R)-3-hydroxy-1-oxo-1-(dodecylamino)butan-2-yl)-3-phenyl acrylic acid amide, (E)-N-((2S,3R)-3-hydroxy-1-oxo-1-(tetradecylamino)butan-2-yl)-3-phenyl acrylic acid amide, (E)-N-((2S,3R)-3-hydroxy-1-oxo-1-(octadecylamino)butan-2-yl)-3-phenyl acrylic acid amide, (E)-N-((2S,3R)-3-hydroxy-1-oxo-1-(octadecylamino)butan-2-yl)-3-phenyl acrylic acid amide, (E)-N-((2S,3R)-3-hydroxy-1-oxo-1-(octadecylamino)butan-2-yl)-3-phenyl acrylic acid amide, N-((2S,3R)-3-hydroxy-1-oxo-1-(octadecylamino)butan-2-yl)-indole-3-acetic acid amide, N-((2S,3R)-3-hydroxy-1-oxo-1-(dodecylamino)butan-2-yl)-isoquinoline-1-carboxamide, N-((2S,3R)-3-hydroxy-1-oxo-1-(tetradecylamino)butan-2-yl)-isoquinoline-1-carboxamide, N-((2S,3R)-3-hydroxy-1-oxo-1-(tetradecylamino)butan-2-yl)-isoquinoline-1-carboxamide, N-((2S,3R)-3-hydroxy-1-oxo-1-(dodecylamino)butan-2-yl)-2-thiophene carboxamide, N-((2S,3R)-3-hydroxy-1-oxo-1-(tetradecylamino)butan-2-yl)-2-thiophene carboxamide, N-((2S,3R)-3-hydroxy-1-oxo-1-(octadecylamino)butan-2-yl)-2-thiophene carboxamide, N-((2S,3R)-3-hydroxy-1-oxo-1-(dodecylamino)butan-2-yl)-benzofuran-2-carboxamide, N-((2S,3R)-3-hydroxy-1-oxo-1-(tetradecylamino)butan-2-yl)-benzofuran-2-carboxamide, N-((2S,3R)-3-hydroxy-1-oxo-1-(octadecylamino)butan-2-yl)-benzofuran-2-carboxamide, N-((2S,3R)-3-hydroxy-1-oxo-1-(octylamino)butan-2-yl)-benzamide, N-((2S,3R)-3-hydroxy-1-oxo-1-(octylamino)butan-2-yl)-benzamide, (2E,4E)-N-((2S,3R)-3-hydroxy-1-oxo-1-(octylamino)butan-2-yl)-2,4-hexadienoic acid amide, (2E,4E)-N-((2S,3R)-3-hydroxy-1-oxo-1-(octylamino)butan-2-yl)-2,4-hexadienoic acid amide, (2E,4E)-N-((2S,3R)-3-hydroxy-1-oxo-1-(octylamino)butan-2-yl)-2,4-hexadienoic acid amide, (2E,4E)-N-((2S,3R)-3-hydroxy-1-oxo-1-(octylamino)butan-2-yl)-2,4-hexadienoic acid amide, N-((2S,3R)-3-hydroxy-1-oxo-1-(octylamino)butan-2-yl)-benzofuran-2-carboxamide, (2S,3R)-2-Amino-3-hydroxy-N-tetradecylbutanamide, (2S,3R)-2-Amino-3-hydroxy-N-tetradecylbutanamide, (2S,3R)-2-Amino-3-hydroxy-N-tetradecylbutanamide, (2S,3R)-2-Amino-3-hydroxy-N-tetradecylbutanamide, N-((2S,3R)-3-hydroxy-1-oxo-1-(dodecylamino)butan-2-yl)-(N-methyl)indole-2-carboxamide, N-((2S,3R)-3-hydroxy-1-oxo-1-(tetradecylamino)butan-2-yl)-(N-methyl)indole-2-carboxamide, N-((2S,3R)-3-hydroxy-1-oxo-1-(octadecylamino)butan-2-yl)-(N-methyl), N-((2S,3R)-3-hydroxy-1-oxo-1-(dodecylamino)butan-2-yl)-6-hydroxylnaphthoic-2-carboxamide, N-((2S,3R)-3-hydroxy-1-oxo-1-(tetradecylamino)butan-2-yl)-6-hydroxylnaphthoic-2-carboxamide, N-((2S,3R)-3-hydroxy-1-oxo-1-(octadecylamino)butan-2-yl)-6-hydroxylnaphthoic-2-carboxamide, N-((2S,3R)-3-hydroxy-1-oxo-1-(dodecylamino)butan-2-yl)-4-methylbenzsulfonamide, N-((2S,3R)-3-hydroxy-1-oxo-1-(tetradecylamino)butan-2-yl)-4-methylbenzsulfonamide, N-((2S,3R)-3-hydroxy-1-oxo-1-(octadecylamino)butan-2-yl)-4-methylbenzsulfonamide, N-((2S,3R)-3-hydroxy-1-oxo-1-(dodecylamino)butan-2-yl)-2-thiophene sulfonamide, N-((2S,3R)-3-hydroxy-1-oxo-1-(dodecylamino)butan-2-yl)-2-thiophene sulfonamide, N-((2S,3R)-3-hydroxy-1-oxo-1-(octadecylamino)butan-2-yl)-2-thiophene sulfonamide, N-((2S,3R)-3-hydroxy-1-oxo-1-(tetradecylamino)butan-2-yl)-2-naphthylsulfonamide, N-((2S,3R)-3-hydroxy-1-oxo-1-(tetradecylamino)butan-2-yl)-2-naphthylsulfonamide, and (3S,4R,5S)-3,4,5-trihydroxy-N-((2S,3R)-3-hydroxy-1-oxo-1-(tetradecylamino)butan-2-yl)cyclohex-1-enecarboxamide.

6. A method of synthesizing a compound of claim 2 comprising combining, adding into, or mixing one or more organic acids with a compound having the following structure: 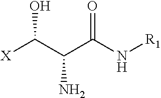
or a derivative thereof, wherein
X is hydrogen, methyl, alkyl, or substituted alkyl;
and R₁ is alkyl or substituted alkyl.

7. A method of making a compound of claim 2 comprising:
a) combining, adding into, or mixing one or more primary amines with one or more compounds having the following formula: 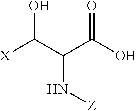
to form an intermediate wherein
X is hydrogen, methyl, alkyl, or substituted alkyl; and
Z is an amine-protecting group;
b) removing the amine-protecting group Z; followed by
c) combining, adding into, or mixing one or more organic acids with the intermediate.

8. A method of synthesizing a compound of claim 2 comprising:
a) attaching one or more amine-protecting groups to an amino acid to form a protected amino acid;
b) combining, adding into, or mixing one or more primary amines with the protected amino acid to form an intermediate;
c) removing the amine-protecting group; followed by
c) combining, adding into, or mixing one or more organic acids with the intermediate to form the compound.

9. A method of inducing apoptosis in a target cell comprising contacting the target cell with one or more compounds of claim 2.

10. A method of treating cancer in a patient in need thereof comprising administering a therapeutically effective amount of one or more compounds of claim 2.

11. A pharmaceutical formulation comprising one or more compounds of claim 2.

12. The formulation of claim 11 further comprising one or more death receptor agonists.

13. The formulation of claim 12 wherein the death receptor agonist is TRAIL.

14. A method of treating cancer in a patient in need thereof comprising administering one or more formulations of claim 11.
